(19) United States
(12) Patent Application Publication
(10) Pub. No.: US 2021/0030758 A1
(43) Pub. Date: Feb. 4, 2021

(54) TREATMENT OF CANCERS HAVING DRIVING ONCOGENIC MUTATIONS

(71) Applicant: G1 Therapeutics, Inc., Research Triangle Park, NC (US)

(72) Inventors: Jay Copeland Strum, Hillsborough, NC (US); Daniel M. Freed, Cary, NC (US); Jessica A. Sorrentino, Durham, NC (US); John E. Bisi, Apex, NC (US); Andrew Beelen, Research Triangle Park, NC (US); Patrick Joseph Roberts, Durham, NC (US)

(73) Assignee: G1 Therapeutics, Inc., Research Triangle Park, NC (US)

(21) Appl. No.: 17/067,549

(22) Filed: Oct. 9, 2020

Related U.S. Application Data
(63) Continuation of application No. PCT/US2019/026656, filed on Apr. 9, 2019.
(60) Provisional application No. 62/810,802, filed on Feb. 26, 2019.
(60) Provisional application No. 62/788,024, filed on Jan. 3, 2019.
(60) Provisional application No. 62/657,373, filed on Apr. 13, 2018.
(60) Provisional application No. 62/655,135, filed on Apr. 9, 2018.

(51) Int. Cl. A61K 31/519; A61P 35/00; A61K 31/4439; A61K 31/506; A61K 31/4184; A61K 31/4523; A61K 31/4545; A61K 31/4745; A61K 31/5377; A61K 31/517; A61K 31/685

(52) U.S. Cl. CPC A61K 31/519; A61P 35/00; A61K 31/4439; A61K 31/506; A61K 31/4184; A61K 31/685; A61K 31/4545; A61K 31/4745; A61K 31/5377; A61K 31/517; A61K 31/4523

(57) ABSTRACT

Methods and compositions are described to treat a cancer having a specific oncogenic driving mutation by administering a CDK4/6 inhibitor in combination with an additional kinase inhibitor, wherein the specific combination provides advantageous or synergistic inhibitory activity, delays acquired resistance to the additional kinase inhibitor, and/or extends the efficacy of the kinase inhibitor.

### CROSS-REFERENCE TO RELATED APPLICATIONS

This application is a continuation of International Patent Application No. PCT/US2019/026656, filed in the Patent Cooperation Treaty, U.S. Receiving Office on Apr. 9, 2019, which claims the benefit of and priority to U.S. Provisional Application No. 62/655,135, filed Apr. 9, 2018, U.S. Provisional Application No. 62/657,373, filed Apr. 13, 2018, U.S. Provisional Application No. 62/788,024, filed Jan. 3, 2019, and U.S. Provisional Application No. 62/810,802, filed Feb. 26, 2019. The entirety of each of these applications is hereby incorporated by reference herein for all purposes.

### FIELD OF THE INVENTION

This invention provides methods and compositions for treating cancers having a specific oncogenic driving mutation with a CDK4/6 inhibitor paired with an additional kinase inhibitor, wherein the specific combination provides advantageous or synergistic inhibitory activity, delays acquired resistance to the additional kinase inhibitor, and/or extends the efficacy of the kinase inhibitor.

### BACKGROUND OF THE INVENTION

Cancer is driven largely by somatic mutations that accumulate in the genome over an individual's lifetime, with additional contributions from epigenetic and transcriptomic alterations. These somatic mutations range in scale from single-nucleotide variants (SNVs), insertions and deletions of a few to a few dozen nucleotides (indels), larger copy-number aberrations (CNAs) and large-genome rearrangements, also called structural variants (SVs) (see Raphael et al., Identifying driver mutations in sequenced cancer genomes: computational approaches to enable precision medicine. Genome Med. 2014; 6(1): 5). Oncogenic driver mutations refer to mutations that are responsible for both the initiation and maintenance of the cancer (see Stratton et al., The cancer genome. Nature 2009, 458(7239):719-724). These mutations are often found in genes that encode for signaling proteins that are critical for maintaining normal cellular proliferation and survival.

The presence of mutations on these genes lead to constitutive activation of mutant signaling proteins that induce and sustain tumorigenesis, and confer growth advantage on cancer cells, favoring their selection during tumor progression.

Over the past decade, it has become evident that subsets of cancers can be further defined at the molecular level by recurrent driver mutations that occur in multiple oncogenes. For example, in non-small cell lung carcinoma (NSCLC), a number of driver mutations have been identified, including AKT1, anaplastic lymphoma kinase (ALK), BRAF, epidermal growth factor receptor (EGFR), ERBB2, KRAS, MEK1, MET, NRAS, PIK3CA, RET, and ROS1. Furthermore, a deeper understanding of the pathobiology of these driver mutations has led to the development of small molecules that target specific driver mutations. In many subsets of cancer, driver mutations are mutually exclusive. For example, EGFR, KRAS, and ALK driver mutations are mutually exclusive in patients with NSCLC, and the presence of one mutation in lieu of another can significantly influence response to targeted therapy. Accordingly, testing for these mutations and tailoring therapy accordingly is widely accepted as standard practice (Kitai et al., Epithelial-to-Mesenchymal Transition Defines Feedback Activation of Receptor Tyrosine Kinase Signaling Induced by MEK Inhibition in KRAS-Mutant Lung Cancer. Cancer Discov. 2016; 6(7); 754-69).

Nonetheless, strategies to inhibit driver mutant proteins or exploit synthetic lethal interactions with a mutant gene have been widely pursued but have been fraught with technical challenges or produced inconsistent results (see, e.g., Ostrem J M, Peters U, Sos M L, Wells J A, Shokat K M. K-Ras(G12C) inhibitors allosterically control GTP affinity and effector interactions. Nature. 2013; 503:548-551; Stephen et al., Dragging ras back in the ring. Cancer cell. 2014; 25:272-281; Cox et al., Drugging the undruggable RAS: Mission possible? Nat Rev Drug Discov. 2014; 13:828-851). Where successful inhibition of driver mutations has been accomplished, effective long-term therapies have been limited due to either toxicities associated with their sustained inhibition (see, e.g., Lito et al., Tumor adaptation and resistance to RAF inhibitors. Nature medicine. 2013; 19:1401-1409; Samatar et al., Targeting RAS-ERK signalling in cancer: promises and challenges. Nat Rev Drug Discov. 2014; 13:928-942) or problems associated with feedback reactivation of signaling pathways, which frequently drives adaptive resistance (see Kitai et al., Epithelial-to-Mesenchymal Transition Defines Feedback Activation of Receptor Tyrosine Kinase Signaling Induced by MEK Inhibition in KRAS-Mutant Lung Cancer. Cancer Discov. 2016; 6(7); 754-69; Sun et al., Intrinsic resistance to MEK inhibition in KRAS mutant lung and colon cancer through transcriptional induction of ERBB3. Cell Rep. 2014; 7(1):86-93; Manchado et al., A combinatorial strategy for treating KRAS-mutant lung cancer. Nature. 2016; 534(7609): 647-51).

Accordingly, effective therapeutic strategies with limited toxicities targeting driver mutations remains a major challenge in treating mutant cancers.

It is an object of the present invention to provide methods and treatments which effectively target driver mutations without unacceptable toxic side-effects.

In addition, it is an object of the present invention to safely and effectively reduce or delay the development of acquired resistance to kinase inhibitors that target driver mutations with a therapeutic regime capable of long-term administration.

### SUMMARY OF THE INVENTION

The present invention provides advantageous methods and compositions for treating a subject having a cancer with a defined driving oncogenic mutation, which includes administering an effective amount of a selective CDK 4/6 inhibitor described herein in combination with an additional kinase inhibitor. The specific combination of a select kinase inhibitor in combination with the selective CDK 4/6 inhibitor described herein provides significant advantageous or synergistic inhibition of tumor growth and progression, which increases therapeutic effectiveness and prevents or delays the acquisition of acquired resistance. By incorporating the selective CDK4/6 inhibitors described herein, the select combinations provide efficacious anti-cancer treatments capable of long-term administration with limited toxicities.

In one aspect of the present invention, a selective CDK4/6 inhibitor selected from the group consisting of:

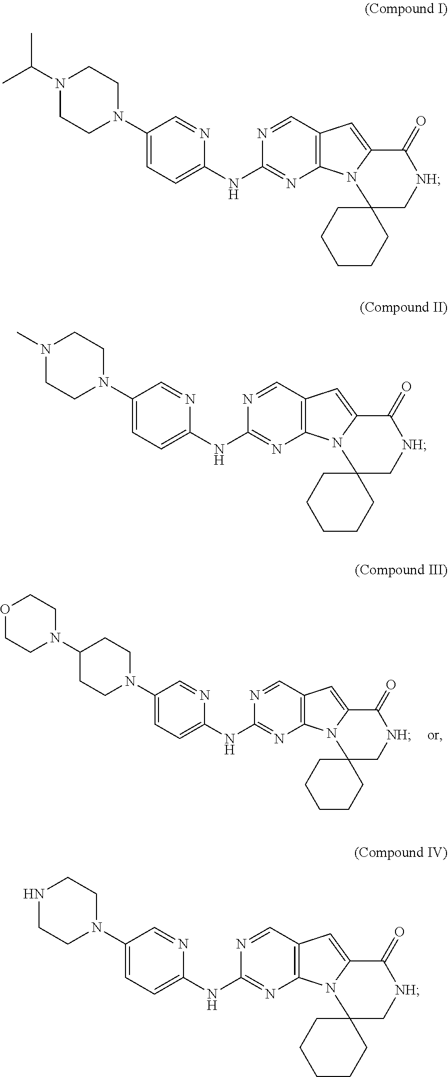 [SMILES: CC(C)N1CCN(c2ccc(Nc3ncc4cc5n(c4n3)C3(CCCCC3)CNC5=O)nc2)CC1 ; CN1CCN(c2ccc(Nc3ncc4cc5n(c4n3)C3(CCCCC3)CNC5=O)nc2)CC1 ; O=C1NCC2(CCCCC2)n2c1cc1cnc(Nc3ccc(N4CCC(N5CCOCC5)CC4)cn3)nc12 ; O=C1NCC2(CCCCC2)n2c1cc1cnc(Nc3ccc(N4CCNCC4)cn3)nc12]

or a pharmaceutically acceptable salt, isotopic analog, or prodrug thereof, optionally in a pharmaceutically acceptable carrier to form a composition, in combination or alternation with at least one additional tumor kinase inhibitor is administered to a subject with a cancer having an identified driver mutation. Compounds I-IV are described in, for example, WO 2012/061156.

In some embodiments, the selective CDK4/6 inhibitor administered in combination or alternation with at least one additional tumor kinase inhibitor to a subject with a cancer having an identified driver mutation is a hydrochloride salt of Compound I, such as the mono- or dihydrochloride salt. In some embodiments, the hydrochloride salt of Compound I is a dihydrochloride salt having the structure:

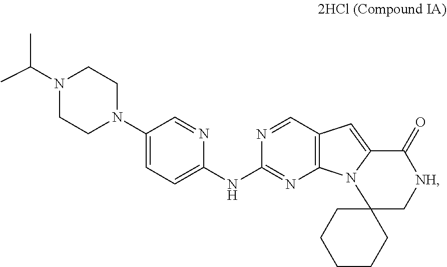 [SMILES: CC(C)N1CCN(c2ccc(Nc3ncc4cc5n(c4n3)C3(CCCCC3)CNC5=O)nc2)CC1]

or a pharmaceutically acceptable composition or isotopic analog thereof. In some embodiments, Compound IA is an isolated morphic form referred to herein as Form B (Compound IA Form B). Compound IA Form B has previously been described in International Patent Publication WO 2019/006393 to G1 Therapeutics, Inc.

In some embodiments, the cancer has a driver mutation of KRAS, EGFR, BRAF, MET, ERBB2, ALK, RET, NRAS, or PIK3CA. In some embodiments, the cancer is a CDK4/6-replication dependent cancer—i.e., requires the activity of CDK 4/6 for replication or proliferation or which may be growth inhibited through the activity of a selective CDK 4/6 inhibitor. In some embodiments, the cancer is a CDK4/6-replication independent cancer, that is does not require the activity of CDK 4/6 for replication or proliferation and may not be growth inhibited through, for example, the activity of a CDK 4/6 inhibitor alone. In some embodiments, the selective CDK4/6 inhibitor is administered to the subject after the subject's cancer has developed resistance to a previous kinase inhibitor therapy. In some embodiments, the selective CDK4/6 inhibitor administered is selected from Compound I, Compound IA, and Compound IA Form B. In some embodiments, the CDK4/6 inhibitor administered is Compound IA Form B.

It has been surprisingly found that the use of a selective CDK4/6 inhibitor described herein in combination with a tumor kinase inhibitor can provide advantageous or synergistic anti-tumor activity even in cancers that are CDK4/6-replication independent, that is does not require the activity of CDK 4/6 for replication or proliferation and may not be growth inhibited through, for example, the activity of a selective CDK 4/6 inhibitor alone. In certain embodiments, provided herein is a method for treating a CDK4/6-replication independent cancer using a CDK4/6 inhibitor described herein in combination with an ALK inhibitor or an ERK inhibitor. In some embodiments, the CDK4/6-replication independent cancer is a retinoblastoma negative (Rb-negative) or Rb-null non-small cell lung cancer (NSCLC). In some embodiments, the Rb-null NSCLC has an ALK rearrangement. In some embodiments, the ALK rearrangement is an EML4-ALK rearrangement. In some embodiments, the subject is administered a selective CDK4/6 inhibitor selected from Compound I-IV, and an ALK inhibitor, for example but not limited to crizotinib or alectinib. In some embodiments, the selective CDK4/6 inhibitor administered is selected from Compound I, Compound IA, and Compound IA Form B. In some embodiments, the selective CDK4/6 inhibitor administered is Compound IA Form B. As shown in Example 2 (FIG. 3), it has been surprisingly shown that including a CDK4/6 inhibitor in combination with an ALK inhibitor in Rb-null NSCLC cell lines with EML4-ALK rearrangements provides an advantageous or synergistic inhibition of growth, even though treatment with the selective CDK4/6 inhibitor alone does not significantly reduce cell growth.

In some embodiments, the Rb-null NSCLC has a MET gene mutation, for example but not limited to an exon 14 deletion, and the subject is administered a selective CDK4/6 inhibitor selected from Compound I-IV, and an ALK inhibitor, for example but not limited to crizotinib or alectinib. In an alternative embodiment, the subject with the Rb-null NSCLC having a MET gene mutation is administered a selective CDK4/6 inhibitor selected from Compound I-IV in combination with an ERK inhibitor, for example but not limited to ulixertinib. In some embodiments, the selective CDK4/6 inhibitor administered is selected from Compound I, Compound IA, and Compound IA Form B. In some embodiments, the selective CDK4/6 inhibitor administered is Compound IA Form B. As shown in Example 2 (FIG. 3), it has been surprisingly shown that including a selective CDK4/6 inhibitor in combination with an ALK inhibitor or ERK inhibitor in Rb-null NSCLC cell lines with a MET gene mutation provides an advantageous or synergistic inhibition of growth, even though treatment with the CDK4/6 inhibitor alone does not significantly reduce cell growth.

In some embodiments, a method is provided of treating CDK4/6 replication independent NSCLC comprising administering a CDK4/6 inhibitor as described herein in combination with a tumor kinase inhibitor selected from a BRAF inhibitor, a MEK inhibitor, an ERK inhibitor, a PI3K inhibitor, an EGFR inhibitor, an ALK inhibitor, and a RET inhibitor. In some embodiments, the tumor kinase inhibitor is a BRAF inhibitor, for example but not limited to dabrafenib. In some embodiments, the tumor kinase inhibitor is a MEK inhibitor, for example but not limited to selumetinib. In some embodiments, the tumor kinase inhibitor is an ERK inhibitor, for example but not limited to ulixertinib. In some embodiments, the tumor kinase inhibitor is a PI3K inhibitor, for example but not limited to dactolisib. In some embodiments, the tumor kinase inhibitor is an EGFR inhibitor, for example but not limited to osimertinib or lapatinib. In some embodiments, the tumor kinase inhibitor is an ALK inhibitor, for example but not limited to crizotinib or alectinib. In some embodiments, the tumor kinase inhibitor is a RET inhibitor, for example but not limited to pralsetinib or agerafenib. In some embodiments, the selective CDK4/6 inhibitor administered is selected from Compound I, Compound IA, and Compound IA Form B. In some embodiments, the selective CDK4/6 inhibitor administered is Compound IA Form B. It has been surprisingly shown that including a selective CDK4/6 inhibitor in combination with one of the above tumor kinase inhibitors provides an advantageous or synergistic inhibition of growth, even though inhibition with th CDK4/6 inhibitor alone does not significantly inhibit growth.

In some embodiments, a method is provided of treating NSCLC with a CCDC6-RET fusion comprising administering a CDK4/6 inhibitor described herein in combination with an ALK inhibitor, for example but not limited to crizotinib or alectinib. In some embodiments, the selective CDK4/6 inhibitor administered is selected from Compound I, Compound IA, and Compound IA Form B. In some embodiments, the selective CDK4/6 inhibitor administered is Compound IA Form B. As shown in Example 2 (FIG. 3), it has been surprisingly shown that including a selective CDK4/6 inhibitor in combination with an ALK inhibitor in NSCLC cells lines with a CCDC6-RET fusion provides an advantageous or synergistic inhibition of growth.

In some embodiments, a method is provided of treating NSCLC with a SLC34A2-ROS1 fusion comprising administering a CDK4/6 inhibitor described herein in combination with an ALK inhibitor, for example but not limited to crizotinib or alectinib. In some embodiments, the selective CDK4/6 inhibitor administered is selected from Compound I, Compound IA, and Compound IA Form B. In some embodiments, the selective CDK4/6 inhibitor administered is Compound IA Form B. As shown in Example 2 (FIG. 3), it has been surprisingly shown that including a selective CDK4/6 inhibitor in combination with an ALK inhibitor in NSCLC cell lines with a SLC34A2-ROS1 fusion provides an advantageous or synergistic inhibition of growth.

In some embodiments, a method is provided of treating NSCLC with a MET amplification comprising administering a CDK4/6 inhibitor described herein in combination with an ALK inhibitor, for example but not limited to crizotinib. In some embodiments, the selective CDK4/6 inhibitor administered is selected from Compound I, Compound IA, and Compound IA Form B. In some embodiments, the selective CDK4/6 inhibitor administered is Compound IA Form B. As shown in Example 2, it has been surprisingly shown that including a selected CDK4/6 inhibitor in combination with an ALK inhibitor in NSCLC cell lines with a MET amplification provides an advantageous or synergistic inhibition of growth.

In some embodiments, a method is provided of treating NSCLC with an NRAS mutation, for example but not limited to an NRAS Q61K substitution, comprising administering a CDK4/6 inhibitor described herein in combination with a MEK inhibitor or a PI3K inhibitor. In some embodiments, the CDK4/6 inhibitor is administered in combination with a MEK inhibitor, for example selumetinib. In some embodiments, the CDK4/6 inhibitor is administered in combination with a PI3K inhibitor, for example dactolisib. In some embodiments, the selective CDK4/6 inhibitor administered is selected from Compound I, Compound IA, and Compound IA Form B. In some embodiments, the selective CDK4/6 inhibitor administered is Compound IA Form B. As shown in Example 2, it has been surprisingly shown that including a selected CDK4/6 in combination with a MEK inhibitor or PI3K inhibitor in NSCLC cell lines with an NRAS Q61K mutation provides an advantageous or synergistic inhibition of growth.

In some embodiments, a method is provided of treating NSCLC with a CCDC6-RET fusion comprising administering a CDK4/6 inhibitor described herein in combination with a RET inhibitor, for example but not limited to pralsetinib or agerafenib. In some embodiments, the selective CDK4/6 inhibitor administered is selected from Compound I, Compound IA, and Compound IA Form B. In some embodiments, the selective CDK4/6 inhibitor administered is Compound IA Form B. As shown in Examples 6 and 7, it has been surprisingly shown that including a selective CDK4/6 inhibitor in combination with a RET inhibitor is NSCLC with a CCDC6-RET fusion provides an advantageous or synergistic inhibition of growth.

In some embodiments, a method is provided of treating NSCLC with a KRAS G12S substitution comprising administering a CDK4/6 inhibitor described herein in combination with selumetinib and ulixertinib. In some embodiments, the selective CDK4/6 inhibitor administered is selected from Compound I, Compound IA, and Compound IA Form B. In some embodiments, the selective CDK4/6 inhibitor administered is Compound IA Form B. As shown in Example 4, it has been surprisingly shown that including a selective CDK4/6 inhibitor in combination with selumetinib and ulixertinib in NSCLC with a KRAS G12S substitution provides an advantageous or synergistic inhibition of growth.

In one aspect, the selective CDK 4/6 inhibitors of Formulas I-IV above can be administered in combination with an additional kinase inhibitor for the treatment of the cancers described herein in a manner that may allow daily treatment of the patient without a drug holiday or without serious gastrointestinal problems. The CDK 4/6 inhibitors described herein for use in combination with an additional kinase inhibitor are short-acting with short half-lives (less than about 16 hours) with non-limiting side-effects, thus allowing their inclusion in a long-term treatment regime without the need for treatment holidays. Furthermore, by using these particular CDK 4/6 inhibitors, therapy-limiting side effects such as neutropenia and gastrointestinal complications associated with other CDK4/6 inhibitors are avoided, and potential treatment limiting side-effect stacking associated with combining a CDK 4/6 inhibitor with an additional kinase inhibitor in a combination treatment is significantly reduced. The CDK 4/6 inhibitors described herein are particularly useful in therapeutic regimens requiring long-term treatment, as required for many kinase inhibitor treatment in, for example NSCLC, while minimizing the effect of CDK4/6 inhibitory toxicity on CDK4/6 replication dependent healthy cells, such as hematopoietic stem cells and hematopoietic progenitor cells (together referred to as HSPCs), and allow for continuous, daily dosing. In some embodiments, the CDK4/6 inhibitor administered is selected from Compound I, Compound IA, and Compound IA Form B. In some embodiments, the CDK4/6 inhibitor administered is Compound IA Form B.

In a particular aspect, the present invention provides methods for treating a subject with cancer that is intrinsically resistant to dabrafenib, selumetinib, ulixertinib, dactolisib, crizotinib, alectinib, or lapatinib kinase inhibitor treatment or has developed acquired resistance to dabrafenib, selumetinib, ulixertinib, dactolisib, crizotinib, alectinib, or lapatinib kinase inhibitor treatment by administering to the patient an effective amount of a selective CDK 4/6 inhibitor described herein in combination with an effective amount of dabrafenib, selumetinib, ulixertinib, dactolisib, crizotinib, alectinib, or lapatinib in a continuous treatment regime without toxic side-effects. In an alternative aspect, the present invention provides methods for treating a subject with cancer that is intrinsically resistant to encorafenib, vemurafenib, idelalisib, copanlisib, taselisib, perifosine, buparlisin, duvelisib, alpelisib, umbralisib, ceritinib, brigatinib, trametinib, cobimetinib, binimetinib, lorlatinib, entrectinib, or SCH772984 kinase inhibitor treatment or has developed acquired resistance to encorafenib, vemurafenib, idelalisib, copanlisib, taselisib, perifosine, buparlisin, duvelisib, alpelisib, umbralisib, ceritinib, brigatinib, trametinib, cobimetinib, binimetinib, lorlatinib, entrectinib, or SCH772984 kinase inhibitor treatment by administering to the patient an effective amount of a selective CDK 4/6 inhibitor described herein in combination with an effective amount of encorafenib, vemurafenib, idelalisib, copanlisib, taselisib, perifosine, buparlisin, duvelisib, alpelisib, umbralisib, ceritinib, brigatinib, trametinib, cobimetinib, binimetinib, lorlatinib, entrectinib, or SCH772984 in a continuous treatment regimen without toxic side effects. It particular, the use of the short-acting selective CDK 4/6 inhibitors described herein in combination with these kinase inhibitors may be efficacious in sensitizing mutant cancer to the inhibitory effects of the additional kinase inhibitor. Furthermore, the administration of a CDK 4/6 inhibitor described herein in combination with an additional kinase inhibitor described herein may delay the onset of resistance to the administered additional kinase inhibitor. Finally, by incorporating a CDK4/6 inhibitor described herein in combination with a kinase inhibitor described herein—wherein the mutant cancer has previously acquired resistance to the additional kinase inhibitor—may reestablish the sensitivity of the cancer to the inhibitory effects of the additional kinase inhibitor. Accordingly, the methods described herein drastically expand the population of cancer patients responsive to initial kinase inhibitor inhibition and extend the efficacy of current kinase inhibitor treatments against mutant cancers by both delaying acquired resistance, re-sensitizing previously resistant tumors to the inhibitory effects of the kinase inhibitor, and increasing the efficacy of the treatment due to the advantageous or synergistic effects of the combination. In some embodiments, the CDK4/6 inhibitor administered is selected from Compound I, Compound IA, and Compound IA Form B. In some embodiments, the CDK4/6 inhibitor administered is Compound IA Form B.

The administration regime for use in the present invention may include daily dosing of both a kinase inhibitor described herein and the CDK 4/6 inhibitor. For example, the kinase inhibitor described herein may be administered at least once a day with the CDK 4/6 inhibitor. Alternatively, the kinase inhibitor described herein may be administered once a day and the CDK 4/6 inhibitor dosed at least once a day, for example once a day, twice a day, or three times a day. Because the CDK 4/6 inhibitors described herein are highly tolerable, the therapeutic regime can be dosed continuously without the need for a drug holiday, further extending the combinations beneficial effects. Accordingly, provided herein is a method of treatment by administering a CDK 4/6 inhibitor described herein in combination with a kinase inhibitor described herein, wherein the combination is administered continuously, for example at least 21 days, at least 28 days, at least 35 days, at least 45 days, at least 56 days, at least 70 days, at least 85 days, at least 102 days, at least 120 days, at least 150 days, at least 204 days, or more, without the need for a scheduled drug holiday. In some embodiments, the CDK4/6 inhibitor administered is selected from Compound I, Compound IA, and Compound IA Form B. In some embodiments, the CDK4/6 inhibitor administered is Compound IA Form B.

In one aspect of the present invention, a method of treating a subject having a cancer with a KRAS mutation is provided wherein the subject is administered a CDK 4/6 inhibitor described herein in combination with selumetinib, dactolisib, or ulixertinib, or a combination thereof. In some embodiments, the subject is administered a CDK 4/6 inhibitor described herein in combination with selumetinib and ulixertinib. In an alternative aspect, a method of treating a subject having cancer with a KRAS mutation is provided wherein the subject is administered a CDK 4/6 inhibitor described herein in combination with encorafenib, vemurafenib, idelalisib, copanlisib, taselisib, perifosine, buparlisib, duvelisib, alpelisib, trametinib, cobimetinib, binimetinib, SCH772984 or umbralisib, or a combination thereof. In some embodiments, the KRAS mutation encodes a G12D substitution, G12C substitution, G12S substitution, Q61K substitution, or G12V substitution, and the additional kinase inhibitor administered in combination with the CDK 4/6 inhibitor is selumetinib. In some embodiments, the KRAS mutation encodes a G12D substitution, G12C substitution, G12S substitution, Q61K substitution, or G12V substitution, and the additional kinase inhibitor administered in combination with the CDK 4/6 inhibitor is ulixertinib. In some embodiments, the KRAS mutation encodes a G12D substitution, G12C substitution, G12S substitution, Q61K substitution, or G12V substitution, and the additional kinase inhibitor in combination with the CDK 4/6 inhibitor is dactolisib. In some embodiments, the cancer harbors a KRAS mutation encoding a G12V substitution and is also retinoblastoma protein (Rb)-negative. In some embodiments, the CDK 4/6 inhibitor administered is compound I. In some embodiments, the KRAS mutant cancer is of the lung, pancreas, colon, colorectal, uterus, stomach, testis, or cervix. In some embodiments, the cancer is NSCLC. In some embodiments, the NSCLC is an epithelial NSCLC. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B. In an alternative embodiment, the KRAS mutation encodes a G12A substitution.

In one aspect of the present invention, a method of treating a subject having a cancer with an EGFR mutation is provided wherein the subject is administered a CDK 4/6 inhibitor described herein in combination with dactolisib or ulixertinib, or a combination thereof. In some embodiments, the subject is administered a CDK 4/6 inhibitor described herein in combination with dactolisib or ulixertinib, and further osimertinib. In an alternative embodiment, a method of treating a subject having a cancer with a EGFR mutation is provided wherein the subject is administered a CDK 4/6 inhibitor described herein in combination with idelalisib, copanlisib, taselisib, perifosine, buparlisib, duvelisib, alpelisib, SCH772984, or umbralisib, or a combination thereof. In some embodiments, the EGFR mutation is an exon 19 deletion, L858 substitution, L858/T790M substitution, or exon 20 insertion and the additional kinase inhibitor administered in combination with the CDK 4/6 inhibitor is ulixertinib. In some embodiments, the EGFR mutation is an exon 19 deletion, L858 substitution, L858/T790M substitution, or exon 20 insertion and the additional kinase inhibitor administered in combination with the CDK 4/6 inhibitor is dactolisib.

In some embodiments, the EGFR mutation is an exon 19 deletion, and the cancer further includes a MET amplification. In some embodiments, the CDK 4/6 inhibitor administered is compound I. In some embodiments, the EGFR mutant cancer is of the bladder, a glioma including glioblastoma, head and neck, breast, cervix, colon and/or colorectal, gastroesophageal, lung, prostate, ovary, pancreas, kidney, thyroid, or squamous cell. In some embodiments, the cancer is NSCLC or breast cancer. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In one aspect of the present invention, a method of treating a subject having a cancer with a BRAF mutation is provided wherein the subject is administered a CDK 4/6 inhibitor described herein in combination with dabrafenib, selumetinib, or ulixertinib, or a combination thereof. In some embodiments, the subject is administered a CDK 4/6 inhibitor described herein in combination with selumetinib and ulixertinib. In some embodiments, the subject is administered a CDK 4/6 inhibitor described herein in combination with dabrafenib and trametinib. In an alternative embodiment, a method of treating a subject having a cancer with a BRAF mutation is provided wherein the subject is administered a CDK 4/6 inhibitor described herein in combination with encorafenib, SCH772984, trametinib, cobimetinib, binimetinib, or vemurafenib, or a combination thereof. In some embodiments, the BRAF mutation encodes a L597V substitution. In some embodiments, the cancer further includes an NRAS Q61K protein substitution. In some embodiments, the BRAF mutation encodes a G466V substitution and the additional kinase inhibitor is dabrafenib. In some embodiments, the BRAF mutation encodes a G469A substitution and the additional kinase inhibitor in combination with the CDK 4/6 inhibitor is ulixetinib. In some embodiments, the BRAF mutation encodes a V600E substitution and the additional kinase inhibitor administered in combination with the CDK4/6 inhibitor is dabrafenib, in further combination with trametinib. In some embodiments, the CDK 4/6 inhibitor administered is Compound I. In some embodiments, the BRAF mutant cancer is thyroid cancer, melanoma, colorectal cancer, lung cancer, uterine cancer, stomach cancer, lymphoma, or bladder cancer, ovarian cancer, glioma and gastrointestinal stromal tumor. In some embodiments, the BRAF mutant cancer is NSCLC or melanoma. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In one aspect of the present invention, a method of treating a subject having a cancer with a MET amplification or mutation is provided wherein the subject is administered a CDK 4/6 inhibitor described herein in combination with ulixertinib, dactolisib, or crizotinib, or a combination thereof. In an alternative embodiment, a method of treating a subject having a cancer with a MET amplification or mutation is provided wherein the subject is administered a CDK 4/6 inhibitor described herein in combination with idelalisib, copanlisib, taselisib, perifosine, buparlisib, duvelisib, alpelisib, umbralisib, ceritinib, brigatinib, SCH772984, lorlatinib, or entrectinib, or a combination thereof. In some embodiments, the cancer has a MET mutation that encodes for an exon 14 deletion mutant. In some embodiments, the cancer has a MET mutation that encodes for an exon 14 deletion mutant and is Rb-protein negative. In some embodiments, the cancer has a MET amplification, and the additional kinase inhibitor administered in combination with the CDK 4/6 inhibitor is dactolisib or crizotinib. In some embodiments, the CDK 4/6 inhibitor administered is Compound I. In some embodiments, the MET mutant cancer is renal cell carcinoma, head and neck squamous cell carcinoma hepatocellular carcinoma, NSCLC, small cell lung cancer, gastric cancer, esophageal cancer, colorectal cancer, gliomas, and ovarian cancer. In some embodiments, the MET mutant cancer is NSCLC or renal cell carcinoma. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In one aspect of the present invention, a method of treating a subject having a cancer with a ERBB2 amplification or mutation is provided wherein the subject is administered a CDK 4/6 inhibitor described herein in combination with ulixertinib, dactolisib, or lapatinib, or a combination thereof. In some embodiments, the cancer has a ERBB2 amplification, and the additional kinase inhibitor administered in combination with the CDK 4/6 inhibitor is lapatinib or ulixertinib. In some embodiments, the cancer has a ERBB2 amplification, and the additional kinase inhibitor administered in combination with the CDK 4/6 inhibitor is lapatinib, in further combination with trastuzumab. In an alternative embodiment, a method of treating a subject having a cancer with a ERBB2 amplification or mutation is provided wherein the subject is administered a CDK 4/6 inhibitor described herein in combination with idelalisib, copanlisib, taselisib, perifosine, buparlisib, duvelisib, alpelisib, SCH772984, or umbralisib, or a combination thereof. In some embodiments, the ERBB2 mutation encodes for a exon 20 insertion, and the additional kinase inhibitor in combination with the CDK 4/6 inhibitor is ulixertinib or dactolisib. In some embodiments, the cancer is ovarian, breast, or NSCLC. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In one aspect of the present invention, a method of treating a subject having a cancer with a ALK gene mutation is provided wherein the subject is administered a CDK 4/6 inhibitor described herein in combination with ulixertinib, crizotinib, or alectinib. In an alternative embodiment, a method of treating a subject having cancer with an ALK gene mutation is provided wherein the subject is administered a CDK 4/6 inhibitor described herein in combination with ceritinib, brigatinib, SCH772984, lorlatinib, or entrectinib, or a combination thereof. In one aspect of the present invention, a method of treating a subject having a cancer with a ALK gene mutation is an ALK fusion. In some embodiments, the ALK fusion is an EML4-ALK fusion. In some embodiments, the ALK fusion is a KIFSB-ALK fusion. In some embodiments, the ALK fusion is a TFG-ALK fusion. In some embodiments, the cancer is also RB-protein negative. In some embodiments, the cancer is NSCLC. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In one aspect of the present invention, a method of treating a subject having a cancer with a ROS1 gene mutation is provided wherein the subject is administered a CDK 4/6 inhibitor described herein in combination with crizotinib. In an alternative embodiment, a method of treating a subject having a cancer with a ROS1 gene mutation is provided wherein the subject is administered a CDK 4/6 inhibitor described herein in combination with ceritinib, brigatinib, lorlatinib, or entrectinib, or a combination thereof. In one aspect of the present invention, a method of treating a subject having a cancer with a ROS1 gene mutation is a ROS1 gene fusion. In some embodiments, the ROS1 fusion is SLC34A2-ROS1. In some embodiments, the ROS1 fusion is CD74-ROS1. In some embodiments, the ROS1 fusion is EZR-ROS1. In some embodiments, the ROS1 fusion is TPM3-ROS1. In some embodiments, the ROS1 fusion is SDC4-ROS1. In some embodiments, the cancer is NSCLC or cholangiocarcinoma. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In one aspect of the present invention, a method of treating a subject having a cancer with a RET gene mutation is provided wherein the subject is administered a CDK 4/6 inhibitor described herein in combination with alectinib, agrefanib, or pralsetinib. In an alternative embodiment, a method of treating a subject having a cancer with a RET gene mutation is provided wherein the subject is administered a CDK 4/6 inhibitor described herein in combination with ceritinib, brigatinib, lorlatinib, or entrectinib, or a combination thereof. In one aspect of the present invention, a method of treating a subject having a cancer with a RET gene mutation is RET gene fusion. In some embodiments, the RET fusion is a CCDC6-RET fusion. In some embodiments, the RET fusion is a KIFSB-RET fusion. In some embodiments, the RET fusion is a TRIM33-RET fusion. In some embodiments, the RET fusion cancer is NSCLC. In some embodiments, the RET fusion mutant cancer is thyroid cancer. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In one aspect of the present invention, a method of treating a subject having a cancer with a NRAS gene mutation is provided wherein the subject is administered a CDK 4/6 inhibitor described herein in combination with dactolisib. In an alternative embodiment, a method of treating a subject having a cancer with a NRAS gene mutation is provided wherein the subject is administered a CDK 4/6 inhibitor described herein in combination with idelalisib, copanlisib, taselisib, perifosine, buparlisib, duvelisib, alpelisib, or umbralisib, or a combination thereof. In some embodiments, the NRAS mutation encodes for a Q61K substitution. In some embodiments, the NRAS mutation encodes for a Q61L, Q61R, or Q61H substitution. In some embodiments, the NRAS mutation encodes for a G12 C, G12R, G12S, G12A, or G12D substitution. In some embodiments, the NRAS cancer is melanoma, hepatocellular carcinoma, myeloid leukemias, NSCLC, or thyroid carcinoma. In some embodiments, the NRAS mutant cancer is NSCLC. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In one aspect of the present invention, a method of treating a subject having a cancer with a PIK3CA gene mutation is provided wherein the subject is administered a CDK 4/6 inhibitor described herein in combination with dabrafenib, selumetinib, ulixertinib, or dactolisib, or a combination thereof. In an alternative embodiment, a method of treating a subject having a cancer with a PIK3CA gene mutation is provided wherein the subject is administered a CDK 4/6 inhibitor described herein in combination with encorafenib, vemurafenib, idelalisib, copanlisib, taselisib, perifosine, buparlisib, duvelisib, alpelisib, trametinib, cobimetinib, binimetinib, SCH772984, or umbralisib, or a combination thereof. In some embodiments, the PIK3CA gene mutation encodes for an E542G substitution and the kinase inhibitor administered in combination with the CDK 4/6 inhibitor is dabrafenib, selumetinib, or ulixertinib, or a combination thereof. In some embodiments, the PIK3CA gene mutation encodes for a H1047R substitution and the kinase inhibitor administered in combination with the CDK 4/6 inhibitor is selumitinib or dactolisib. In some embodiments, the PIK3CA gene mutation encodes for a G106-R108 deletion and the kinase inhibitor administered in combination with the CDK 4/6 inhibitor is dactolisib. In some embodiments, the PIK3CA mutant cancer is also Rb-protein negative. In some embodiments, the PIK3CA gene mutation encodes for a E545Q or H1047L mutation. In some embodiments, the PIK3CA mutant cancer is a colon cancer, glioma, gastric cancer, breast cancer, endometrial cancer, or lung cancer. In some embodiments, the cancer is NSCLC. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

Also provided herein is a composition comprising a CDK4/6 inhibitor described herein in a combined dosage form with one or more additional kinase inhibitors selected from dabrafenib, selumetinib, ulixertinib, dactolisib, crizotinib, alectinib, pralsetinib, or lapatinib. In some embodiments, the CDK4/6 inhibitor is selected from Compound I, Compound IA, and Compound IA Form B. In some embodiments, the CDK4/6 inhibitor is Compound IA Form B.

### BRIEF DESCRIPTION OF THE FIGURES

FIG. 1 is a bar graph showing the percent inhibition of tumor growth in various NSCLC PDX models with varying genetic mutations when treated with Compound I (100 mg/kg). The y axis is tumor growth inhibition measured in percent. The x-axis is the NSCLC PDX model. The dotted line is the responder/non-responder cutoff (58% TGI) for correlation analysis).

FIG. 2 is a bar graph showing the IC₅₀ of Compound I in various lung cancer cell lines with varying oncogenic mutations. The y-axis is the IC₅₀ of Compound measured in micromolar concentration. The x-axis is the specific lung cancer cell type.

FIG. 3 is a heat map showing synergism of Compound I and various inhibitors targeting specific oncogenic drivers in various NSCLC cell lines. The y-axis is the kinase inhibitor used in combination. The top x-axis is the cell line, and the bottom x-axis is the oncogenic mutation associated with that cell line.

FIG. 4A is an immunoblot showing the effect of Compound I (0.5 μM), selumetinib (1 μM), and/or ulixertinib (1 μM) alone or in combination on pRB1 (S807/811), RB1, cyclin D1, pErk1/2 (T202/Y204), pRSK (S380), Bim, and Survin levels in A549 NSCLC cells. Alpha-tubulin was used as a loading control.

FIG. 4B is an immunoblot showing the effect of Compound 1 (0.25 μM) and/or crizotinib (1 μM) alone or in combination on pRB1 (S807/811), RB1, cyclin D1, pErk1/2 (T202/Y204), pRSK (S380), Bim, and Survin levels in H3122 NSCLC cells. Alpha-tubulin was used as a loading control.

FIG. 5A, FIG. 5B, and FIG. 5C are scatter plots showing the effect of Compound I (100 mg/kg), selumetinib (50 mg/kg), and/or twice daily doses of ulixertinib (50 mg/kg) in an A549 human NSCLC xenograft model. The y-axis is tumor volume in mm³ at the end of treatment. The x-axis is the provided treatment. Error bars represent standard error of the mean. Data were analyzed using a two-tailed Student's t test comparing the targeted agent to the targeted agent+Compound 1. *p≤0.05; ***p≤0.001.

FIG. 6A is a scatter plot showing the effect of Compound I (100 mg/kg) and/or crizotinib (25 mg/kg for the first 12 days, then 50 mg/kg) alone or in combination in a H3122 human NSCLC xenograft model. The y-axis is tumor volume in mm³ at the end of treatment. The x-axis is the provided treatment. Error bars represent standard error of the mean. Data were analyzed using a two-tailed Student's t test comparing the targeted agent to the targeted agent+Compound I. *p≤0.05; ***p≤0.001.

FIG. 6B is a graph showing the effect of vehicle, Compound I (50 mg/kg), crizotinib (20 mg/kg), or the combination of Compound I (50 mg/kg) and crizotinib (20 mg/kg) administered to mice bearing EML4-ALK NSCLC PDX tumor.

FIG. 7 is a graph showing the effect of Compound 1, pralsetinib, and Compound 1 (0.3 μM)+pralsetinib on LC2/ad non-small cell lung cancer cells expressing an oncogenic RET fusion. The x-axis is the logarithmic inhibitor concentration expressed in moles per liter. The y-axis is cell viability measured as a % of control.

FIG. 8 is a graph showing the effect of Compound 1, agerafenib, and Compound 1 (0.3 μM)+agerafenib on LC2/ad non-small cell lung cancer cells expressing an oncogenic RET fusion. The x-axis is the logarithmic inhibitor concentration expressed in moles per liter. The y-axis is cell viability measured as a % of control.

FIG. 9 contains images of well plates containing LC2/ad non-small cell lung cancer cells expressing an oncogenic RET fusion 14 days after being treated with DMSO, 300 nM of pralsetinib, 300 nM of Compound 1, or pralsetinib+Compound 1. The plates were stained with crystal violet.

FIG. 10 contains images of well plates containing LC2/ad non-small cell lung cancer cells expressing an oncogenic RET fusion 21 and 28 days after being treated with 300 nM of pralsetinib or pralsetinib+300 nM Compound 1. The plates were stained with crystal violet.

FIG. 11 is a bar graph that shows the absorbance of solubilized crystal violet stain for LC2/ad non-small cell lung cancer cells treated with vehicle (DMSO), 300 nM Compound 1, 300 nM pralsetinib, or Compound 1+pralsetinib after 14, 21, and 28 days. The x-axis measures time in days. The y-axis measured absorbance at 562 nm. *p<0.05, ***p=0.0001, ****p<0.0001 using two-way ANOVA.

FIG. 12 is a comparison of XRPD patterns of Form A, Form B, and Form C. These three forms were obtained from crystallization and slurry experiments as described in Example 9 and shown in Tables 1-4. The x-axis is 2Theta measured in degrees and the y-axis is intensity measured in counts.

FIG. 13 is a comparison of XRPD patterns of Form D, Form E, and Form F. These three forms were obtained from crystallization and slurry experiments as described in Example 9 and shown in Tables 1-4. The x-axis is 2Theta measured in degrees and the y-axis is intensity measured in counts.

FIG. 14 is a comparison of XRPD patterns of Form G and Form H. These two forms were obtained from crystallization and slurry experiments as described in Example 9 and shown in Tables 1-4. Form G is an anhydrate and Form H is an n-PrOH solvate. The x-axis is 2Theta measured in degrees and the y-axis is intensity measured in counts.

FIG. 15 is the XRPD pattern for pure Form B. The peaks, marked with bars, are listed in Example 10. The x-axis is 2Theta measured in degrees and the y-axis is intensity measured in counts.

FIG. 16 are XRPD patterns of Form I and Form J. The x-axis is 2Theta measured in degrees and the y-axis is intensity measured in counts.

### DETAILED DESCRIPTION OF THE INVENTION

### Definitions

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as it if were individually recited herein. The endpoints of all ranges are included within the range and independently combinable. All methods described herein can be performed in a suitable order unless otherwise indicated herein or otherwise clearly contradicted by context. The use of examples, or exemplary language (e.g. “such as”), is intended merely to better illustrate the invention and does not pose a limitation on the scope of the invention unless otherwise claimed.

The term “carrier” applied to pharmaceutical compositions/combinations of the invention refers to a diluent, excipient, or vehicle with which a CDK4/6 inhibitor described herein is provided.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like.

The term “pharmaceutically acceptable salt” as used herein refers to those salts which are, within the scope of sound medical judgment, suitable for use in contact with subjects (e.g., human subjects) without undue toxicity, irritation, allergic response, and the like, commensurate with a reasonable benefit/risk ratio, and effective for their intended use, as well as the zwitterionic forms, where possible, of the compounds of the presently disclosed subject matter.

Thus, the term “salt” refers to the relatively non-toxic, inorganic and organic acid addition salts of compounds of the presently disclosed subject matter. These salts can be prepared in situ during the final isolation and purification of the compounds or by separately reacting the purified compound in its free base form with a suitable organic or inorganic acid and isolating the salt thus formed. Pharmaceutically acceptable base addition salts may be formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

The compound of the present invention may form a solvate with solvents (including water). Therefore, in one non-limiting embodiment, the invention includes a solvated form of the compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a compound of the invention and water. Pharmaceutically acceptable solvates in accordance with the invention include those wherein the solvent may be isotopically substituted, e.g. D20, d₆-acetone, d₆-DMSO. A solvate can be in a liquid or solid form.

“Pharmaceutical compositions” are compositions comprising at least one active agent, and at least one other substance, such as a carrier. “Pharmaceutical combinations” are combinations of at least two active agents which may be combined in a single dosage form or provided together in separate dosage forms with instructions that the active agents are to be used together to treat any disorder described herein.

A “pharmaceutically acceptable excipient” means an excipient that is useful in preparing a pharmaceutical composition/combination that is generally safe, non-toxic and neither biologically nor otherwise inappropriate for administration to a host, typically a human. In some embodiments, an excipient is used that is acceptable for veterinary use.

In some embodiments, Compounds I-IV includes desired isotopic substitutions of atoms, at amounts above the natural abundance of the isotope, i.e., enriched. Isotopes are atoms having the same atomic number but different mass numbers, i.e., the same number of protons but a different number of neutrons. By way of general example and without limitation, isotopes of hydrogen, for example, deuterium (²H) and tritium (³H) may be used anywhere in described structures. Alternatively, or in addition, isotopes of carbon, e.g., ¹³C and ¹⁴C, may be used. A preferred isotopic substitution is deuterium for hydrogen at one or more locations on the molecule to improve the performance of the drug. The deuterium can be bound in a location of bond breakage during metabolism (an α-deuterium kinetic isotope effect) or next to or near the site of bond breakage (a β-deuterium kinetic isotope effect).

Substitution with isotopes such as deuterium can afford certain therapeutic advantages resulting from greater metabolic stability, such as, for example, increased in vivo half-life or reduced dosage requirements. Substitution of deuterium for hydrogen at a site of metabolic break down can reduce the rate of or eliminate the metabolism at that bond. At any position of the compound that a hydrogen atom may be present, the hydrogen atom can be any isotope of hydrogen, including protium (¹H), deuterium (²H) and tritium (³H). Thus, reference herein to a compound encompasses all potential isotopic forms unless the context clearly dictates otherwise.

The term “isotopically-labeled” analog refers to an analog that is a “deuterated analog”, a “¹³C-labeled analog,” or a “deuterated/¹³C-labeled analog.” The term “deuterated analog” means a compound described herein, whereby a H-isotope, i.e., hydrogen/protium (¹H), is substituted by a H-isotope, i.e., deuterium (²H). Deuterium substitution can be partial or complete. Partial deuterium substitution means that at least one hydrogen is substituted by at least one deuterium. In certain embodiments, the isotope is 90%, 95%, or 99% or more enriched in an isotope at any location of interest. In some embodiments it is deuterium that is 90%, 95%, or 99% enriched at a desired location.

In the description below and herein generally, whenever any of the terms referring to Compounds I-IV are used, it should be understood that pharmaceutically acceptable salts or compositions are considered included, unless otherwise stated or inconsistent with the text.

Compounds are described using standard nomenclature. Unless defined otherwise, all technical and scientific terms used herein have the same meaning as is commonly understood by one of skill in the art to which this invention belongs.

The “patient” or “subject” treated is typically a human patient, although it is to be understood the methods described herein are effective with respect to other animals, such as mammals. More particularly, the term patient can include animals used in assays such as those used in preclinical testing including but not limited to mice, rats, monkeys, dogs, pigs and rabbits; as well as domesticated swine (pigs and hogs), ruminants, equine, poultry, felines, bovines, murines, canines, and the like.

The term “selective CDK4/6 inhibitor” used in the context of the compounds described herein includes compounds that inhibit CDK4 activity, CDK6 activity, or both CDK4 and CDK6 activity at an IC50 molar concentration at least about 500, or 1000, or 1500, or 1800, or 2000 times less than the IC50 molar concentration necessary to inhibit to the same degree of CDK2 activity in a standard phosphorylation assay.

In some embodiments, the term “CDK4/6-replication dependent cancer” refers to a cancer or cellular proliferation disorder that requires the activity of CDK4/6 for replication or proliferation, or which may be growth inhibited through the activity of a selective CDK4/6 inhibitor. Cancers and disorders of such a type can be characterized by (e.g., that has cells that exhibit) the present of a functional Retinoblastoma protein. Such cancers and disorders are classified as being “Rb-positive”. Rb-positive abnormal cellular proliferation disorders, and variations of this term as used herein, refer to disorders or diseases caused by uncontrolled or abnormal cellular division which are characterized by the presence of a functional Retinoblastoma protein, which can include cancers.

In some embodiments, the term “CDK4/6-replication independent cancer” refers to a cancer that does not significantly require the activity of CDK4/6 for replication. Cancers of such type are often, but not always, characterized by (e.g. has cells that exhibit), but are not limited to: increased activity of cyclin-dependent kinase 1 (CDK1); increased activity of cyclin-dependent kinase 2 (CDK2); loss, deficiency, or absence of retinoblastoma tumor suppressor protein (Rb)(Rb-null); high levels of p16Ink4a expression; high levels of MYC expression; increased expression of cyclin E1, cyclin E2, and cyclin A; and combinations thereof. The cancer may be characterized by reduced expression of the retinoblastoma tumor suppressor protein or a retinoblastoma family member protein or proteins (such as, but not limited to p107 and p130). In certain embodiments, a tumor or cancer that is CDK4/6-replication independent is a tumor or cancer whose cell population, as a whole, does not experience substantial G1 cell-cycle arrest when exposed to a selective CDK4/6 inhibitor. In certain embodiments, a tumor or cancer that is CDK4/6-replication independent is a tumor or cancer who has a cell population wherein less than 25%, 20%, 15%, 10%, or 5% of its cells experience G1 cell-cycle arrest when exposed to a selective CDK4/6 inhibitor.

“Intrinsic resistance,” also known as primary resistance, as used herein, refers to a condition wherein a cancer is not responsive to the initial inhibitory effects of an administered treatment.

“Acquired resistance,” as used herein, refers to a condition wherein a cancer that was or is initially sensitive to the inhibitory effects of an inhibitor compound becomes non-responsive or less-responsive over time to the effects of that compound. Without wishing to be bound by any one theory, it is believed that acquired resistance to an inhibitor occurs due to one or more additional mutations or genetic alterations in bypass signaling that develops after the onset of inhibitor treatment. In certain embodiments, a tumor or cancer that has acquired resistance to an inhibitor is a tumor or cancer who has a cell population wherein less than 50%, 40%, 30% 20%, 15%, 10%, or 5% of its cells experience inhibitor, leading to disease progression.

As contemplated herein and for purposes of the disclosed ranges herein, all ranges described herein include any and all numerical values occurring within the identified ranges. For example, a range of 1 to 10, or between 1 and 10, as contemplated herein, would include the numerical values 1, 2, 3, 4, 5, 6, 7, 8, 9, 10, as well as fractions thereof.

Compounds are described using standard nomenclature. Unless defined otherwise, all technical and scientific terms used herein have the same meaning as is commonly understood by one of skill in the art to which this invention belongs.

### CDK4/6 Inhibitors

CDK4/6 inhibitors for use in the present invention include Compounds I-IV or a pharmaceutically acceptable salt, composition, isotopic analog, or prodrug thereof. Compounds I-IV are described in WO 2012/061156, incorporated herein by reference in its entirety. In some embodiments, Compound I is administered as a dihydrochloride salt (Compound IA). In some embodiments, Compound IA is administered in the Form B morphic form (Compound IA Form B) as described in WO 2019/006393, incorporated herein by reference in its entirety.

### Isolated Morphic Form B of Compound IA

Compound IA Form B is a highly stable, highly crystalline form of solid Compound I, which is beneficial for therapeutic efficacy and for the manufacture of pharmaceutical formulations. Form B is stable under thermal stress of 60° C. for 7 days. Additionally, a long-term stability study at 25° C. and 60% relative humidity revealed that isolated Compound IA Form B is stable for at least 1 year. In one embodiment isolated Compound IA Form B is stable for at least about 6, 7, 8, 9, 10, 11, 12, 14, 16, 18, 20, 22, or 24 months.

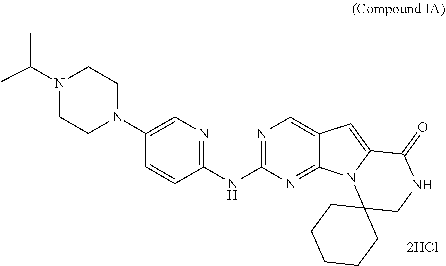 [SMILES: CC(C)N1CCN(c2ccc(Nc3ncc4cc5n(c4n3)C3(CCCCC3)CNC5=O)nc2)CC1]

A number of crystallization and slurry experiments were conducted by varying temperature, cooling procedure, and isolation procedure. From these experiments, eleven unique forms of Compound IA were discovered, but only Form A, Form B, and Form D were appropriate for evaluation. The other forms resulted in weak crystalline forms, solvates, unstable hydrates, or anhydrates. Of the three solid forms, Form B was discovered to be an unexpectedly superior highly crystalline stable material for therapeutic dosage forms. In the dynamic vapor sorption experiment, Compound IA remained in Form B after exposure to 90% relative humidity.

Form B has advantageous properties for use as an active pharmaceutical ingredient in a solid dosage form and may have increased efficacy in such a formulation. Form B can be produced by recrystallization from HCl and acetone, as described in more detail below. Form B is characterized by an XRPD pattern substantially similar to that set forth in FIG. 17. In some embodiments, Form B is characterized by an XRPD pattern comprising at least three 2theta values selected from 6.5°±0.2, 9.5±0.2°, 14.0±0.2°, 14.4±0.2°, 18.1±0.2°, 19.7±0.2°, and 22.4±0.2°. In some embodiments, Form B is characterized by an XRPD pattern comprising at least the 2theta values of 9.5±0.2°. In some embodiments isolated Compound IA, Form B is characterized by the absence of at least one of the peaks at 4.6±0.2° 2theta. In some embodiments isolated Compound IA, Form B is characterized by the absence of a peak at 5.0±0.2° 2theta. In some embodiments, isolated Form B is characterized as having a 7.5% weight loss between 31 and 120° C. in a thermogravimetric infrared (TG-IR) analysis. In some embodiments, isolated Form B is characterized as having differential scanning calorimetry (DSC) onset endotherms at about 105±20° C., about 220±20° C., and about 350±20° C., for example at 105° C., 220° C., and 350° C. or 92° C., 219° C., and 341° C.

Compound IA Form B can be produced, for example, by recrystallizing Compound I in concentrated HCl and acetone. In some embodiments, Compound I is dissolved in concentrated HCl and heated. This is followed by the addition of acetone and isolation of the product by cooling and filtration.

In some embodiments, Compound IA Form B is produced by the recrystallization of Compound IA Form D. In an alternative embodiment, Compound IA Form B is produced by repeated recrystallizations. In some embodiments, pure Compound IA Form B is purified from impure Compound IA Form B by a water:acetone (1:2) (v/v) slurry followed by vacuum drying.

Compound IA Form A has less stability than Form B. Form A was produced when MeOH, EtOH, and 1-BuOH were used as solvents in the single solvent crystallizations and it was also produced in the binary solvent crystallizations using water and MeOH as the primary solvent. Slurry experiments using n-heptane and c-hexane produced Form A as well.

Compound IA Form D has less stability than Form B. In some embodiments, Form D is produced by stirring a slurry of Compound IA in acetonitrile at room temperature. In another embodiment, Form D is produced by dissolving Compound I in concentrated HCl before heating. Then the solution is allowed to cool and acetone is only added after crystallization begins to drive the precipitation to completion. The precipitate is then isolated via filtration. In an alternative embodiment, Form D is produced by dissolving Compound I in concentrated HCl before heating. Then the solution is allowed to cool and acetone is only added once crystallization has occurred and all solids are collected via filtration.

In alternative embodiments, a combination of two or more Forms of Compound IA is administered in accordance with the methods described herein, such as Forms B and D; Forms B and A; or Forms A and D. In an alternative embodiment, an isolated combination of three forms is provided, for example, Forms A, B, and D.

### KRAS

The KRAS gene (Entrez 3845) encodes K-Ras that is part of a signaling pathway known as the RAS/MAPK pathway. The K-Ras protein is a GTPase, which means it converts a molecule called GTP into another molecule called GDP. In this way the K-Ras protein acts like a switch that is turned on and off by the GTP and GDP molecules. To transmit signals, it must be turned on by attaching (binding) to a molecule of GTP. The K-Ras protein is turned off (inactivated) when it converts the GTP to GDP. When the protein is bound to GDP, it does not relay signals to the cell's nucleus.

Mutations in the RAS family of proteins are frequently observed across cancer types. The amino acid positions that account for the overwhelming majority of these mutations are G12, G13 and Q61. The different protein isoforms, despite their raw similarity, also behave very differently when expressed in non-native tissue types, likely due to differences in the C-terminal hyper-variable regions. Mis-regulation of isoform expression has been shown to be a driving event in cancer, as well as missense mutations at the three hotspots previously mentioned. While highly recurrent in cancer, attempts to target these RAS mutants with inhibitors have not been successful, and has not yet become common practice in the clinic. The prognostic implications for KRAS mutations vary between cancer types, but have been shown to be associated with poor outcome in colorectal cancer, non-small cell lung cancer, and others.

The KRAS gene belongs to a class of genes known as oncogenes. When mutated, oncogenes have the potential to cause normal cells to become cancerous. The KRAS gene is in the Ras family of oncogenes, which also includes two other genes: HRAS and NRAS. These proteins play important roles in cell division, cell differentiation, and the self-destruction of cells (apoptosis).

In some embodiments, provided is a method of treating a subject having a cancer with a KRAS mutation wherein the subject is administered a CDK 4/6 inhibitor described herein in combination with selumetinib, dactolisib, or ulixertinib, or a combination thereof. In some embodiments, the subject is administered a CDK 4/6 inhibitor described herein in combination with selumetinib and ulixertinib. In an alternative embodiment, provided is a method of treating a subject having a cancer with a KRAS mutation wherein the subject is administered a CDK 4/6 inhibitor described herein in combination with encorafenib, vemurafenib, idelalisib, copanlisib, taselisib, perifosine, buparlisib, duvelisib, alpelisib, trametinib, cobimetinib, binimetinib, SCH772984, or umbralisib, or a combination thereof. In some embodiments, the KRAS mutation encodes a G12D substitution, G12C substitution, or G12V substitution, and the additional kinase inhibitor is selumetinib. In some embodiments, the KRAS mutation encodes a G12S substitution, G12C substitution, Q61K substitution, or G12V substitution, and the additional kinase inhibitor is ulixertinib. In some embodiments, the KRAS mutation encodes a G12D substitution, G12C substitution, Q61K substitution, or G12V substitution, and the additional kinase inhibitor is dactolisib. In some embodiments, the cancer harbors a KRAS mutation encoding a G12V substitution and is also retinoblastoma protein (Rb)-negative. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In some embodiments, the KRAS mutant cancer is selected from pancreatic adenocarcinoma, colon adenocarcinoma, rectum adenocarcinoma, lung adenocarcinoma, uterine corpus endometrial carcinoma, uterine carcinosarcoma, stomach adenocarcinoma, testicular germ cell tumors, cervical squamous cell carcinoma, endocervical adenocarcinoma, cholangiocarcinoma, lymphoid neoplasm diffuse large B-cell lymphoma, acute myeloid leukemia, bladder urothelial carcinoma, skin cutaneous melanoma, lung squamous cell carcinoma, kidney renal papillary cell carcinoma, liver hepatocellular carcinoma, esophageal carcinoma, ovarian serous cystadenocarcinoma, and sarcoma. In some embodiments, the KRAS mutant cancer is of the lung, pancreas, colon, colorectal, uterus, stomach, testis, or cervix. In some embodiments, the cancer is NSCLC. In an alternative embodiment, the cancer harboring a KRAS mutation encoding a G12D substitution is pancreatic adenocarcinoma, colon adenocarcinoma, rectum adenocarcinoma, or uterine corpus endometrial carcinoma. In some embodiments, the cancer harboring a KRAS mutation encoding a G12C substitution is lung adenocarcinoma or rectum adenocarcinoma. In some embodiments, the cancer harboring a KRAS mutation encoding a G12V substitution is pancreatic adenocarcinoma, rectum adenocarcinoma, colon adenocarcinoma, lung adenocarcinoma, or uterine carcinosarcoma. In some embodiments, the cancer harboring a KRAS mutation encoding a G12S substitution is rectum adenocarcinoma or colon adenocarcinoma. In some embodiments, the cancer harboring a KRAS mutation encoding a Q61K substitution is cholangiocarcinoma or colon adenocarcinoma.

In an alternative embodiment, the subject having a KRAS mutant cancer is administered a CDK 4/6 inhibitor described herein in combination with abemaciclib, abiraterone, afatinib, bosutinib, cabozantinib, dasatinib, enasidenib, enzalutamide, erlotinib, everolimus, erdafitinib, fulvestrant, gefitinib, ibrutinib, imatinib, ipatasertib, lapatinib, nilotinib, niraparib, olaparib, olarutumab, osimertinib, palbociclib, pazopanib, PF7775, Ponatinib, ramucirumab, regorafenib, ribociclib, rucaparib, savolitinib, sorafenib, sunitinib, talazoparib, trastuzumab, trilaciclib, or vistusertib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a KRAS mutant cancer is administered a CDK 4/6 inhibitor described herein in combination with alectinib, alpelisib, binimetinib, brigatinib, ceritinib, cobimetinib, copanlisib, crizotinib, dabrafenib, encorafenib, idelalisib, lorlatinib, SCH772984, selumetinib, trametinib, ulixertinib, or verumarenib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a KRAS mutant cancer is administered a CDK 4/6 inhibitor described herein in combination with a RET inhibitor. In some embodiments, the RET inhibitor is pralsetinib. In some embodiments, the RET inhibitor is agerafenib. In some embodiments, the RET inhibitor is vandentanib. In some embodiments, the RET inhibitor is lenvatinib. In some embodiments, the RET inhibitor is apatanib. In some embodiments, the RET inhibitor is sitravatinib. In some embodiments, the RET inhibitor is LOXO-292. In some embodiments, the CDK4/6 inhibitor administered is selected from Compound I, Compound IA, and Compound IA Form B. In some embodiments, the CDK4/6 inhibitor administered is Compound IA Form B.

### EGFR

The epidermal growth factor receptor gene (EGFR) (Entrez 1956) encodes a protein that belongs to a family of receptor tyrosine kinases (RTKs) that include EGFR/ERBB1, HER2/ERBB2/NEU, HER3/ERBB3, and HER4/ERBB4. The binding of ligands, such as epidermal growth factor (EGF), induces a conformational change that facilitates receptor homo- or heterodimer formation, thereby resulting in activation of EGFR tyrosine kinase activity. Activated EGFR then phosphorylates its substrates, resulting in activation of multiple downstream pathways within the cell, including the PI3K-AKT-mTOR pathway, which is involved in cell survival, and the RAS-RAF-MEK-ERK pathway, which is involved in cell proliferation.

Approximately 10% of patients with NSCLC in the US and 35% in East Asia have tumor associated EGFR mutations (Lynch et al. 2004; Paez et al. 2004; Pao et al. 2004). These mutations occur within EGFR exons 18-21, which encodes a portion of the EGFR kinase domain. EGFR mutations are usually heterozygous, with the mutant allele also showing gene amplification (Soh et al. 2009). Approximately 90% of these mutations are exon 19 deletions or exon 21 L858R point mutations (Ladanyi and Pao 2008). These mutations increase the kinase activity of EGFR, leading to hyperactivation of downstream pro-survival signaling pathways (Sordella et al. 2004). Regardless of ethnicity, EGFR mutations are more often found in tumors from female never smokers (defined as less than 100 cigarettes in a patient's lifetime) with adenocarcinoma histology (Lynch et al. 2004; Paez et al. 2004; Pao et al. 2004). However, EGFR mutations can also be found in other subsets of NSCLC, including in former and current smokers as well as in other histologies. In the vast majority of cases, EGFR mutations are non-overlapping with other oncogenic mutations found in NSCLC (e.g., KRAS mutations, ALK rearrangements, etc.).

In some embodiments, provided is a method of treating a subject having a cancer with a EGFR mutation wherein the subject is administered a CDK 4/6 inhibitor described herein in combination with dactolisib or ulixertinib, or a combination thereof. In some embodiments, the subject is administered a CDK 4/6 inhibitor described herein in combination with dactolisib or ulixertinib, and further osimertinib. In an alternative embodiment, provided is a method of treating a subject having cancer with a EGFR mutation wherein the subject is administered a CDK 4/6 inhibitor described herein in combination with idelalisib, copanlisib, taselisib, perifosine, buparlisib, duvelisib, alpelisib, SCH772984, or umbralisib, or a combination thereof. In some embodiments, the EGFR mutation is an exon 19 deletion or exon 20 insertion and the additional kinase inhibitor is ulixertinib. In some embodiments, the EGFR mutation is an exon 19 deletion, L858 substitution, L858/T790M substitution, exon 20 insertion and the additional kinase inhibitor is dactolisib. In some embodiments, the EGFR mutation is an exon 19 deletion, and the cancer further includes a MET amplification. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In some embodiments, the EGFR mutant cancer is selected from glioblastoma multiforme, lung adenocarcinoma, skin cutaneous melanoma, uterine corpus endometrial carcinoma, brain lower grade glioma, colon adenocarcinoma, stomach adenocarcinoma, cervical squamous cell carcinoma, endocervical adenocarcinoma, ovarian serous cystadenocarcinoma, adrenocortical carcinoma, head and neck squamous cell carcinoma, lung squamous cell carcinoma, bladder urothelial carcinoma, rectum adenocarcinoma, liver hepatocellular carcinoma, breast invasive carcinoma, cholangiocarcinoma, uterine carcinosarcoma, and kidney chromophobe. In some embodiments, the EGFR mutant cancer is of the bladder, a glioma including glioblastoma, head and neck, breast, cervix, colon and/or colorectal, gastroesophageal, lung, prostate, ovary, pancreas, kidney, thyroid, or squamous cell. In some embodiments, the cancer is NSCLC or breast cancer. In an alternative embodiment, the cancer harboring an EGFR mutation encoding a L858R substitution is lung adenocarcinoma. In some embodiments, the cancer harboring an EGFR mutation encoding a T790M substitution is lung adenocarcinoma.

In an alternative embodiment, the subject having a EGFR mutant cancer is administered a CDK 4/6 inhibitor described herein in combination with abemaciclib, abiraterone, afatinib, bosutinib, cabozantinib, dasatinib, enasidenib, enzalutamide, erlotinib, everolimus, erdafitinib, fulvestrant, gefitinib, ibrutinib, imatinib, ipatasertib, lapatinib, nilotinib, niraparib, olaparib, olarutumab, osimertinib, palbociclib, pazopanib, PF7775, Ponatinib, ramucirumab, regorafenib, ribociclib, rucaparib, savolitinib, sorafenib, sunitinib, talazoparib, trastuzumab, trilaciclib, or vistusertib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a EGFR mutant cancer is administered a CDK 4/6 inhibitor described herein in combination with alectinib, alpelisib, binimetinib, brigatinib, ceritinib, cobimetinib, copanlisib, crizotinib, dabrafenib, encorafenib, idelalisib, lorlatinib, SCH772984, selumetinib, trametinib, ulixertinib, or verumarenib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having an EGFR mutant cancer is administered a CDK 4/6 inhibitor described herein in combination with a RET inhibitor. In some embodiments, the RET inhibitor is pralsetinib. In some embodiments, the RET inhibitor is agerafenib. In some embodiments, the RET inhibitor is vandentanib. In some embodiments, the RET inhibitor is lenvatinib. In some embodiments, the RET inhibitor is apatanib. In some embodiments, the RET inhibitor is sitravatinib. In some embodiments, the RET inhibitor is LOXO-292. In some embodiments, the CDK4/6 inhibitor administered is selected from Compound I, Compound IA, and Compound IA Form B. In some embodiments, the CDK4/6 inhibitor administered is Compound IA Form B.

In some embodiments, the subject has NSCLC with an EGFR mutation and at least one non-EGFR mutation that confers resistance to an EGFR-TKI. In some embodiments, the non-EGFR mutation is a MET amplification. In some embodiments, the NSCLC has an EGFR mutation induced by treatment with an EGFR-TKI. In some embodiments, the subject has NSCLC with at least one EGFR mutation and a MET amplification, and is administered a CDK4/6 inhibitor described herein in combination with osimertinib. In some embodiments, the CDK4/6 inhibitor is selected from Compound I, Compound IA, and Compound IA Form B.

### BRAF

The BRAF gene (Entrez 673) encodes a protein—B-Raf—belonging to the raf/mil family of serine/threonine protein kinases. This protein plays a role in regulating the MAP kinase/ERKs signaling pathway, which affects cell division, differentiation, and secretion. Mutations in this gene have been associated with various cancers, including non-Hodgkin lymphoma, colorectal cancer, malignant melanoma, thyroid carcinoma, non-small cell lung carcinoma, adenocarcinoma of lung, ovarian cancer, glioma and gastrointestinal stromal tumor.

In some embodiments, provided is a method of treating a subject having a cancer with a BRAF mutation wherein the subject is administered a CDK 4/6 inhibitor described herein in combination with dabrafenib, selumetinib, or ulixertinib, or a combination thereof. In some embodiments, the subject is administered a CDK 4/6 inhibitor described herein in combination with selumetinib and ulixertinib. In an alternative embodiment, provided is a method of treating a subject having a cancer with a BRAF mutation wherein the subject is administered a CDK 4/6 inhibitor described herein in combination with encorafenib, trametinib, cobimetinib, binimetinib, SCH772984, or vemurafenib, or a combination thereof. In some embodiments, the BRAF mutation encodes a L597V substitution. In some embodiments, the cancer further includes an NRAS Q61K protein substitution. In some embodiments, the BRAF mutation encodes a G466V substitution and the additional kinase inhibitor is dabrafenib. In some embodiments, the BRAF mutation encodes a G469A substitution and the additional kinase inhibitor is ulixetinib. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In some embodiments, the BRAF mutant cancer is selected from thyroid carcinoma, skin cutaneous melanoma, colon adenocarcinoma, uterine corpus endometrial carcinoma, lung adenocarcinoma, stomach adenocarcinoma, rectum adenocarcinoma, bladder urothelial carcinoma, lung squamous cell carcinoma, lymphoid neoplasm diffuse large B-cell lymphoma, glioblastoma multiforme, head and neck squamous cell carcinoma, cholangiocarcinoma, kidney renal papillary cell carcinoma, uterine carcinosarcoma, cervical squamous cell carcinoma, endocervical adenocarcinoma, pancreatic adenocarcinoma, prostate adenocarcinoma, mesothelioma, and adrenocortical carcinoma. In some embodiments, the BRAF mutant cancer is thyroid cancer, melanoma, colorectal cancer, lung cancer, uterine cancer, stomach cancer, lymphoma, or bladder cancer, ovarian cancer, glioma and gastrointestinal stromal tumor. In some embodiments, the BRAF mutant cancer is NSCLC or melanoma. In an alternative embodiment, the cancer harboring a BRAF mutation encoding a L597V substitution is colon adenocarcinoma. In some embodiments, the cancer harboring a BRAF mutation encoding a G469A substitution is pheochromocytoma, paraganglioma, or prostate adenocarcinoma. In some embodiments, the cancer harboring a BRAF mutation encoding a V600E substitution is thyroid carcinoma, skin cutaneous melanoma, or colon adenocarcinoma.

In an alternative embodiment, the subject having a BRAF mutant cancer is administered a CDK 4/6 inhibitor described herein in combination with abemaciclib, abiraterone, afatinib, bosutinib, cabozantinib, dasatinib, enasidenib, enzalutamide, erlotinib, everolimus, erdafitinib, fulvestrant, gefitinib, ibrutinib, imatinib, ipatasertib, lapatinib, nilotinib, niraparib, olaparib, olarutumab, osimertinib, palbociclib, pazopanib, PF7775, Ponatinib, ramucirumab, regorafenib, ribociclib, rucaparib, savolitinib, sorafenib, sunitinib, talazoparib, trastuzumab, trilaciclib, or vistusertib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a BRAF mutant cancer is administered a CDK 4/6 inhibitor described herein in combination with alectinib, alpelisib, binimetinib, brigatinib, ceritinib, cobimetinib, copanlisib, crizotinib, dabrafenib, encorafenib, idelalisib, lorlatinib, SCH772984, selumetinib, trametinib, ulixertinib, or verumarenib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a BRAF mutant cancer is administered a CDK 4/6 inhibitor described herein in combination with a RET inhibitor. In some embodiments, the RET inhibitor is pralsetinib. In some embodiments, the RET inhibitor is agerafenib. In some embodiments, the RET inhibitor is vandentanib. In some embodiments, the RET inhibitor is lenvatinib. In some embodiments, the RET inhibitor is apatanib. In some embodiments, the RET inhibitor is sitravatinib. In some embodiments, the RET inhibitor is LOXO-292. In some embodiments, the CDK4/6 inhibitor administered is selected from Compound I, Compound IA, and Compound IA Form B. In some embodiments, the CDK4/6 inhibitor administered is Compound IA Form B.

### MET

The MET gene (Entrez 4233) (MNNG-HOS transforming gene; Cooper et al. 1984) located on chromosome 7, encodes a receptor tyrosine kinase (RTK) belonging to the MET/RON family of RTKs. Binding of its ligand, hepatocyte growth factor (HGF; also called scatter factor (SF)), induces a conformational change in the MET receptor that facilitates receptor phosphorylation and activation. Activated MET then phosphorylates its substrates, resulting in activation of multiple downstream pathways within the cell, including the PI3K-AKT-mTOR pathway, which is involved in cell survival, and the RAS-RAF-MEK-ERK pathway, which is involved in cell proliferation. In the context of malignancy, aberrant signaling through the MET receptor promotes pleiotrophic effects including growth, survival, invasion, migration, angiogenesis and metastasis (Birchmeier et al. 2003; Peruzzi and Bottaro 2006).

The MET receptor and/or its ligand HGF have been reported to be aberrantly activated in many human cancers. Germline mutations in the tyrosine kinase domain of MET occur in 100% of hereditary papillary renal cell carcinoma, and somatic mutations in MET are found in 10-15% of sporadic papillary renal cell carcinoma (Schmidt et al. 1997). Mutations in MET have been reported at low frequencies in head and neck squamous cell carcinoma (Di Renzo et al. 2000), childhood hepatocellular carcinoma (Park et al. 1999), NSCLC (Kong-Beltran et al. 2006; Ma et al. 2003) and small cell lung cancer (Ma et al. 2003). Amplification of MET has been reported in gastric cancer (Nakajima et al. 1999), esophageal cancer (Miller et al. 2006), colorectal cancer (Umeki, Shiota, and Kawasaki 1999), gliomas (Beroukhim et al. 2007), clear cell ovarian cancer (Yamamoto et al. 2011) and NSCLC (Bean et al. 2007; Cappuzzo et al. 2009; Chen et al. 2009; Engelman et al. 2007; Kubo et al. 2009; Okuda et al. 2008; Onozato et al. 2009).

In some embodiments, provided is a method of treating a subject having a cancer with a MET amplification or mutation wherein the subject is administered a CDK 4/6 inhibitor described herein in combination with ulixertinib, dactolisib, or crizotinib, or a combination thereof. In an alternative embodiment, provided is a method of treating a subject having a cancer with a MET amplification or mutation wherein the subject is administered a CDK 4/6 inhibitor described herein in combination with idelalisib, copanlisib, taselisib, perifosine, buparlisib, duvelisib, alpelisib, umbralisib, ceritinib, brigatinib, SCH772984, lorlatinib, or entrectinib, or a combination thereof. In some embodiments, the cancer has a MET mutation that encodes for an exon 14 deletion mutant. In some embodiments, the cancer has a MET mutation that encodes for an exon 14 deletion mutant and is Rb-protein negative. In some embodiments, the cancer has a MET amplification, and the additional kinase inhibitor is dactolisib or crizotinib. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In some embodiments, the MET mutant cancer is selected from uterine corpus endometrial carcinoma, skin cutaneous melanoma, kidney renal papillary cell carcinoma, bladder urothelial carcinoma, colon adenocarcinoma, lung adenocarcinoma, uterine carcinosarcoma, glioblastoma multiforme, stomach adenocarcinoma, lymphoid neoplasm diffuse large B-cell lymphoma, sarcoma, ovarian serous cystadenocarcinoma, lung squamous cell carcinoma, esophageal carcinoma, kidney renal clear cell carcinoma, acute myeloid leukemia, cervical squamous cell carcinoma, endocervical adenocarcinoma, breast invasive carcinoma, prostate adenocarcinoma, and head and heck squamous cell carcinoma. In some embodiments, the MET mutant cancer is renal cell carcinoma, head and neck squamous cell carcinoma hepatocellular carcinoma, NSCLC, small cell lung cancer, gastric cancer, esophageal cancer, colorectal cancer, gliomas, and ovarian cancer. In some embodiments, the MET mutant cancer is NSCLC or renal cell carcinoma.

In an alternative embodiment, the subject having a MET mutant cancer is administered a CDK 4/6 inhibitor described herein in combination with abemaciclib, abiraterone, afatinib, bosutinib, cabozantinib, dasatinib, enasidenib, enzalutamide, erlotinib, everolimus, erdafitinib, fulvestrant, gefitinib, ibrutinib, imatinib, ipatasertib, lapatinib, nilotinib, niraparib, olaparib, olarutumab, osimertinib, palbociclib, pazopanib, PF7775, Ponatinib, ramucirumab, regorafenib, ribociclib, rucaparib, savolitinib, sorafenib, sunitinib, talazoparib, trastuzumab, trilaciclib, or vistusertib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a MET mutant cancer is administered a CDK 4/6 inhibitor described herein in combination with alectinib, alpelisib, binimetinib, brigatinib, ceritinib, cobimetinib, copanlisib, crizotinib, dabrafenib, encorafenib, idelalisib, lorlatinib, SCH772984, selumetinib, trametinib, ulixertinib, or verumarenib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a MET mutant cancer is administered a CDK 4/6 inhibitor described herein in combination with a RET inhibitor. In some embodiments, the RET inhibitor is pralsetinib. In some embodiments, the RET inhibitor is agerafenib. In some embodiments, the RET inhibitor is vandentanib. In some embodiments, the RET inhibitor is lenvatinib. In some embodiments, the RET inhibitor is apatanib. In some embodiments, the RET inhibitor is sitravatinib. In some embodiments, the RET inhibitor is LOXO-292. In some embodiments, the CDK4/6 inhibitor administered is selected from Compound I, Compound IA, and Compound IA Form B. In some embodiments, the CDK4/6 inhibitor administered is Compound IA Form B.

### ERBB2

ERBB2 (Entrez 2064) encodes a member of the epidermal growth factor (EGF) receptor family of receptor tyrosine kinases. This protein has no ligand binding domain of its own and therefore cannot bind growth factors. However, it does bind tightly to other ligand-bound EGF receptor family members to form a heterodimer, stabilizing ligand binding and enhancing kinase-mediated activation of downstream signaling pathways, such as those involving mitogen-activated protein kinase and phosphatidylinositol-3 kinase. Allelic variations at amino acid positions 654 and 655 of isoform a (positions 624 and 625 of isoform b) have been reported, with the most common allele, Ile654/Ile655, shown here. Amplification and/or overexpression of this gene has been reported in numerous cancers, including breast and ovarian tumors.

In some embodiments, provided is a method of treating a subject having a cancer with an ERBB2 amplification or mutation wherein the subject is administered a CDK 4/6 inhibitor described herein in combination with ulixertinib, dactolisib, or lapatinib, or a combination thereof. In an alternative embodiment, provided is a method of treating a subject having a cancer with an ERBB2 amplification or mutation wherein the subject is administered a CDK 4/6 inhibitor described herein in combination with idelalisib, copanlisib, taselisib, perifosine, buparlisib, duvelisib, alpelisib, SCH772984, or umbralisib, or a combination thereof. In some embodiments, the cancer has an ERBB2 amplification, and the additional kinase inhibitor administered is lapatinib or ulixertinib. In some embodiments, the ERBB2 mutation encodes for an exon 20 insertion, and the additional kinase inhibitor is ulixertinib or dactolisib. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In some embodiments, the cancer with an ERBB2 amplification or mutation is selected from bladder urothelial carcinoma, uterine corpus endometrial carcinoma, colon adenocarcinoma, stomach adenocarcinoma, esophageal adenocarcinoma, cervical squamous cell carcinoma, endocervical adenocarcinoma, rectum adenocarcinoma, skin cutaneous melanoma, cholangiocarcinoma, lung adenocarcinoma, breast invasive carcinoma, lung squamous cell carcinoma, glioblastoma multiforme, head and neck squamous cell carcinoma, ovarian serous cystadenocarcinoma, kidney renal papillary cell carcinoma, uterine carcinosarcoma, thymoma, acute myeloid leukemia, and kidney renal clear cell carcinoma. In some embodiments, the cancer is ovarian, breast, or NSCLC.

In an alternative embodiment, the subject having a ERBB2 mutant cancer is administered a CDK4/6 inhibitor described herein in combination with abemaciclib, abiraterone, afatinib, bosutinib, cabozantinib, dasatinib, enasidenib, enzalutamide, erlotinib, everolimus, erdafitinib, fulvestrant, gefitinib, ibrutinib, imatinib, ipatasertib, lapatinib, nilotinib, niraparib, olaparib, olarutumab, osimertinib, palbociclib, pazopanib, PF7775, Ponatinib, ramucirumab, regorafenib, ribociclib, rucaparib, savolitinib, sorafenib, sunitinib, talazoparib, trastuzumab, trilaciclib, or vistusertib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a ERBB2 mutant cancer is administered a CDK 4/6 inhibitor described herein in combination with alectinib, alpelisib, binimetinib, brigatinib, ceritinib, cobimetinib, copanlisib, crizotinib, dabrafenib, encorafenib, idelalisib, lorlatinib, SCH772984, selumetinib, trametinib, ulixertinib, or verumarenib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a ERBB2 mutant cancer is administered a CDK 4/6 inhibitor described herein in combination with a RET inhibitor. In some embodiments, the RET inhibitor is pralsetinib. In some embodiments, the RET inhibitor is agerafenib. In some embodiments, the RET inhibitor is vandentanib. In some embodiments, the RET inhibitor is lenvatinib. In some embodiments, the RET inhibitor is apatanib. In some embodiments, the RET inhibitor is sitravatinib. In some embodiments, the RET inhibitor is LOXO-292. In some embodiments, the CDK4/6 inhibitor administered is selected from Compound I, Compound IA, and Compound IA Form B. In some embodiments, the CDK4/6 inhibitor administered is Compound IA Form B.

### ALK

Approximately 3-7% of lung tumors harbor ALK fusions (Koivunen et al. 2008; Kwak et al. 2010; Shinmura et al. 2008; Soda et al. 2007; Takeuchi et al. 2008; Wong et al. 2009). ALK fusions are more commonly found in light smokers (<10 pack years) and/or never-smokers (Inamura et al. 2009; Koivunen et al. 2008; Kwak et al. 2010; Soda et al. 2007; Wong et al. 2009). ALK fusions are also associated with younger age (Inamura et al. 2009; Kwak et al. 2010; Wong et al. 2009) and adenocarcinomas with acinar histology (Inamura et al. 2009; Wong et al. 2009) or signet-ring cells (Kwak et al. 2010). Clinically, the presence of EML4-ALK fusions is associated with EGFR tyrosine kinase inhibitor (TKI) resistance (Shaw et al. 2009).

Multiple different ALK rearrangements have been described in NSCLC. The majority of these ALK fusion variants are comprised of portions of the echinoderm microtubule-associated protein-like 4 (EML4) gene with the ALK gene. At least nine different EML4-ALK fusion variants have been identified in NSCLC (Choi et al. 2008; Horn and Pao 2009; Koivunen et al. 2008; Soda et al. 2007; Takeuchi et al. 2008; Takeuchi et al. 2009; Wong et al. 2009). In addition, non-EML4 fusion partners have also been identified, including KIF5B-ALK (Takeuchi et al. 2009) and TFG-ALK (Rikova et al. 2007). Clinically, the presence of an ALK rearrangement is detected by fluorescence in situ hybridization (FISH) with an ALK break apart probe. FISH testing is not able to discern which particular ALK fusion is found in a clinical sample.

In the vast majority of cases, ALK rearrangements are non-overlapping with other oncogenic mutations found in NSCLC (e.g., EGFR mutations, KRAS mutations, etc.; Inamura et al. 2009; Kwak et al. 2010; Shinmura et al. 2008; Wong et al. 2009).

The abnormal EML4-ALK gene fusion leads to the production of a protein (EML4-ALK) that appears, in many cases, to promote and maintain the malignant behavior of the cancer cells (see Soda M, Choi Y L, Enomoto M, et al. (August 2007). “Identification of the transforming EML4-ALK fusion gene in non-small-cell lung cancer”. Nature. 448 (7153): 561-6). The transforming EML4-ALK fusion gene was first reported in non-small cell lung carcinoma (NSCLC) in 2007 (see Sasaki T, Rodig S J, Chirieac L R, Janne P A (July 2010). “The biology and treatment of EML4-ALK non-small cell lung cancer”. Eur. J. Cancer. 46 (10): 1773-80).

In some embodiments, provided is a method of treating a subject with an ALK fusion gene mutation wherein the subject is administered a CDK 4/6 inhibitor described herein in combination with ulixertinib, crizotinib, or alectinib. In an alternative embodiment, provided is a method of treating a subject with an ALK fusion gene mutation wherein the subject is administered a CDK 4/6 inhibitor described herein in combination with ceritinib, brigatinib, SCH772984, lorlatinib, or entrectinib, or a combination thereof. In some embodiments, the ALK fusion is EML4-ALK. In some embodiments, the cancer is also RB-protein negative. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In some embodiments, the cancer with an ALK fusion gene mutation is selected from skin cutaneous melanoma, uterine corpus endometrial carcinoma, colon adenocarcinoma, lung adenocarcinoma, stomach adenocarcinoma, ovarian serous cystadenocarcinoma, lung squamous cell carcinoma, cervical squamous cell carcinoma, endocervical adenocarcinoma, bladder urothelial carcinoma, esophageal carcinoma, head and neck squamous cell carcinoma, rectum adenocarcinoma, adrenocortical carcinoma, liver hepatocellular carcinoma, kidney renal clear cell carcinoma, glioblastoma multiforme, uterine carcinosarcoma, breast invasive carcinoma, kidney chromophome, and acute myeloid leukemia. In some embodiments, the cancer is NSCLC.

In an alternative embodiment, the subject with cancer having a ALK fusion gene mutation is administered a CDK 4/6 inhibitor described herein in combination with abemaciclib, abiraterone, afatinib, bosutinib, cabozantinib, dasatinib, enasidenib, enzalutamide, erlotinib, everolimus, erdafitinib, fulvestrant, gefitinib, ibrutinib, imatinib, ipatasertib, lapatinib, nilotinib, niraparib, olaparib, olarutumab, osimertinib, palbociclib, pazopanib, PF7775, Ponatinib, ramucirumab, regorafenib, ribociclib, rucaparib, savolitinib, sorafenib, sunitinib, talazoparib, trastuzumab, trilaciclib, or vistusertib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject with a cancer having a ALK fusion gene mutation is administered a CDK 4/6 inhibitor described herein in combination with alectinib, alpelisib, binimetinib, brigatinib, ceritinib, cobimetinib, copanlisib, crizotinib, dabrafenib, encorafenib, idelalisib, lorlatinib, SCH772984, selumetinib, trametinib, ulixertinib, or verumarenib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject with a cancer having a ALK fusion gene mutation is administered a CDK 4/6 inhibitor described herein in combination with a RET inhibitor. In some embodiments, the RET inhibitor is pralsetinib. In some embodiments, the RET inhibitor is agerafenib. In some embodiments, the RET inhibitor is vandentanib. In some embodiments, the RET inhibitor is lenvatinib. In some embodiments, the RET inhibitor is apatanib. In some embodiments, the RET inhibitor is sitravatinib. In some embodiments, the RET inhibitor is LOXO-292. In some embodiments, the CDK4/6 inhibitor administered is selected from Compound I, Compound IA, and Compound IA Form B. In some embodiments, the CDK4/6 inhibitor administered is Compound IA Form B.

In some embodiments, the subject has NSCLC having an ALK rearrangement and is administered a CDK4/6 inhibitor in combination with crizotinib. In some embodiments, the CDK4/6 inhibitor administered is selected from Compound I, Compound IA, and Compound IA Form B. In some embodiments, the CDK4/6 inhibitor administered is Compound IA Form B.

### ROS1 Fusion

The ROS1 gene (Entrez 6098) encodes a receptor tyrosine kinase (RTK) of the insulin receptor family. Chromosomal rearrangements involving the ROS1 gene, on chromosome 6q22, were originally described in glioblastomas (e.g., FIG-ROS1; Birchmeier, Sharma, and Wigler 1987; Birchmeier et al. 1990; Charest et al. 2003). More recently, ROS1 fusions were identified as a potential “driver” mutation in non-small cell lung cancer (Rikova et al. 2007) and cholangiocarcinoma (Gu et al. 2011).

ROS1 fusions contain an intact tyrosine kinase domain. To date, those tested biologically possess oncogenic activity (Charest et al. 2003; Rikova et al. 2007). Signaling downstream of ROS1 fusions results in activation of cellular pathways known to be involved in cell growth and cell proliferation. Approximately 2% of lung tumors harbor ROS1 fusions (Bergethon et al. 2012). Several different ROS1 rearrangements have been described in NSCLC. These include SLC34A2-ROS1, CD74-ROS1, EZR-ROS1, TPM3-ROS1, and SDC4-ROS1 (Davies et al. 2012; Rikova et al. 2007; Takeuchi et al. 2012). ROS1 rearrangements are non-overlapping with other oncogenic mutations found in NSCLC (e.g., EGFR mutations, KRAS mutations, ALK fusions, etc.; Bergethon et al. 2012).

In some embodiments, provided is a method of treating a subject with a ROS1 fusion gene mutation wherein the subject is administered a CDK 4/6 inhibitor described herein in combination with crizotinib. In an alternative embodiment, provided is a method of treating a subject with a ROS1 gene mutation wherein the subject is administered a CDK 4/6 inhibitor described herein in combination with ceritinib, brigatinib, lorlatinib, or entrectinib, or a combination thereof. In some embodiments, the ROS1 fusion is SLC34A2-ROS1. In some embodiments, the ROS1 fusion is CD74-ROS1. In some embodiments, the ROS1 fusion is EZR-ROS1. In some embodiments, the ROS1 fusion is TPM3-ROS1. In some embodiments, the ROS1 fusion is SDC4-ROS1. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In some embodiments, the cancer with a ROS1 fusion is selected from skin cutaneous melanoma, uterine corpus endometrial carcinoma, colon adenocarcinoma, lung squamous cell carcinoma, rectum adenocarcinoma, stomach adenocarcinoma, head and neck squamous cell carcinoma, bladder urothelial carcinoma, cervical squamous cell carcinoma, endocervical adenocarcinoma, lung adenocarcinoma, lymphoid neoplasm diffuse large B-cell lymphoma, uterine carcinomasarcoma, kidney renal clear cell carcinoma, esophageal carcinoma, glioblastoma multiforme, ovarian serous cystadenocarcinoma, breast invasive carcinoma, liver hepatocellular carcinoma, adrenocortical carcinoma, and acute myeloid leukemia. In some embodiments, the cancer is NSCLC or cholangiocarcinoma.

In an alternative embodiment, the subject having a cancer with a ROS1 fusion is administered a CDK 4/6 inhibitor described herein in combination with abemaciclib, abiraterone, afatinib, bosutinib, cabozantinib, dasatinib, enasidenib, enzalutamide, erlotinib, everolimus, erdafitinib, fulvestrant, gefitinib, ibrutinib, imatinib, ipatasertib, lapatinib, nilotinib, niraparib, olaparib, olarutumab, osimertinib, palbociclib, pazopanib, PF7775, Ponatinib, ramucirumab, regorafenib, ribociclib, rucaparib, savolitinib, sorafenib, sunitinib, talazoparib, trastuzumab, trilaciclib, or vistusertib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a cancer with a ROS1 fusion is administered a CDK 4/6 inhibitor described herein in combination with alectinib, alpelisib, binimetinib, brigatinib, ceritinib, cobimetinib, copanlisib, crizotinib, dabrafenib, encorafenib, idelalisib, lorlatinib, SCH772984, selumetinib, trametinib, ulixertinib, or verumarenib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a cancer with a ROS1 fusion is administered a CDK 4/6 inhibitor described herein in combination with a RET inhibitor. In some embodiments, the RET inhibitor is pralsetinib. In some embodiments, the RET inhibitor is agerafenib. In some embodiments, the RET inhibitor is vandentanib. In some embodiments, the RET inhibitor is lenvatinib. In some embodiments, the RET inhibitor is apatanib. In some embodiments, the RET inhibitor is sitravatinib. In some embodiments, the RET inhibitor is LOXO-292. In some embodiments, the CDK4/6 inhibitor administered is selected from Compound I, Compound IA, and Compound IA Form B. In some embodiments, the CDK4/6 inhibitor administered is Compound IA Form B.

### RET

The RET gene (Entrez 5979) is located on chromosome 10 and encodes a receptor tyrosine kinase (RTK) belonging to the RET family of RTKs. This gene plays a crucial role in neural crest development. Binding of its ligands, the glial cell line derived neurotrophic factor (GDNF) family of extracellular signaling molecules (Airaksinen, Titievsky, and Saarma 1999), induces receptor phosphorylation and activation. Activated RET then phosphorylates its substrates, resulting in activation of multiple downstream cellular pathways (Phay and Shah 2010).

Genomic alterations in RET are found in several different types of cancer. Activating point mutations in RET can give rise to the hereditary cancer syndrome, multiple endocrine neoplasia 2 (MEN2; Salvatore et al. 2000). Somatic point mutations in RET are also associated with sporadic medullary thyroid cancer (Ciampi and Nikiforov 2007; Salvatore et al. 2000). Oncogenic kinase fusions involving the RET gene are found in ˜1% of non-small cell lung cancers (Pao and Hutchinson 2012).

Approximately 1.3% of lung tumors evaluated have chromosomal changes which lead to RET fusion genes (Ju et al. 2012; Kohno et al. 2012; Takeuchi et al. 2012; Lipson et al. 2012). These gene rearrangements appear to occur almost entirely in adenocarcinoma histology tumors. Histology has not been thoroughly evaluated, but all of the reported lung tumors with RET fusions have been adenocarcinomas (more than 400 lung cancers with histologies other than adenocarcinoma have been tested). Where overlap was evaluated, RET fusions have been shown to occur in tumors without other common driver oncogenes (e.g., EGFR, KRAS, ALK). The three reported fusion genes are CCDC6-RET, KIFSB-RET and TRIM33-RET.

In some embodiments, provided is a method of treating a subject having a cancer with a RET gene mutation wherein the subject is administered a CDK 4/6 inhibitor described herein in combination with alectinib. In an alternative embodiment, provided is a method of treating a subject having a cancer with a RET gene mutation wherein the subject is administered a CDK 4/6 inhibitor described herein in combination with ceritinib, brigatinib, lorlatinib, or entrectinib, or a combination thereof. In one aspect of the present invention, a method of treating a subject having a cancer with a RET gene mutation is RET gene fusion. In some embodiments, the RET fusion is a CCDC6-RET fusion. In some embodiments, the RET fusion is a KIF5B-RET fusion. In some embodiments, the RET fusion is a TRIM33-RET fusion. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In some embodiments, the cancer having a RET gene mutation is selected from uterine corpus endometrial carcinoma, skin cutaneous melanoma, colon adenocarcinoma, lung squamous cell carcinoma, rectum adenocarcinoma, stomach adenocarcinoma, esophageal carcinoma, lung adenocarcinoma, cholangiocarcinoma, pheochromocytoma, paradanglioma, adrenocortical carcinoma, ovarian serous cystadenocarcinoma, head and neck squamous cell carcinoma, bladder urothelial carcinoma, cervical squamous cell carcinoma, endocervical adenocarcinoma, glioblastoma multiforme, brain lower grade glioma, sarcoma, uveal melanoma, and mesothelioma. In some embodiments, the RET fusion cancer is NSCLC. In some embodiments, the RET fusion mutant cancer is thyroid cancer.

In an alternative embodiment, the subject having a cancer with a RET gene mutation is administered a CDK 4/6 inhibitor described herein in combination with abemaciclib, abiraterone, afatinib, bosutinib, cabozantinib, dasatinib, enasidenib, enzalutamide, erlotinib, everolimus, erdafitinib, fulvestrant, gefitinib, ibrutinib, imatinib, ipatasertib, lapatinib, nilotinib, niraparib, olaparib, olarutumab, osimertinib, palbociclib, pazopanib, PF7775, Ponatinib, ramucirumab, regorafenib, ribociclib, rucaparib, savolitinib, sorafenib, sunitinib, talazoparib, trastuzumab, trilaciclib, or vistusertib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a cancer with a RET gene mutation is administered a CDK 4/6 inhibitor described herein in combination with alectinib, alpelisib, binimetinib, brigatinib, ceritinib, cobimetinib, copanlisib, crizotinib, dabrafenib, encorafenib, idelalisib, lorlatinib, SCH772984, selumetinib, trametinib, ulixertinib, or verumarenib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a cancer with a RET gene mutation is administered a CDK 4/6 inhibitor described herein in combination with a RET inhibitor. In some embodiments, the RET gene mutation is a RET fusion. In some embodiments, the RET inhibitor is pralsetinib. In some embodiments, the RET inhibitor is agerafenib. In some embodiments, the RET inhibitor is vandentanib. In some embodiments, the RET inhibitor is lenvatinib. In some embodiments, the RET inhibitor is apatanib. In some embodiments, the RET inhibitor is sitravatinib. In some embodiments, the RET inhibitor is LOXO-292. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having NSCLC with a RET gene fusion is administered a CDK 4/6 inhibitor described herein in combination with a RET inhibitor. In some embodiments, the RET inhibitor is pralsetinib. In some embodiments, the RET inhibitor is agerafenib. In some embodiments, the RET inhibitor is vandentanib. In some embodiments, the RET inhibitor is lenvatinib. In some embodiments, the RET inhibitor is apatanib. In some embodiments, the RET inhibitor is sitravatinib. In some embodiments, the RET inhibitor is LOXO-292. In some embodiments, the CDK4/6 inhibitor administered is selected from Compound I, Compound IA, and Compound IA Form B. In some embodiments, the CDK4/6 inhibitor administered is Compound IA Form B.

### NRAS

Three different human RAS genes have been identified: KRAS (homologous to the oncogene from the Kirsten rat sarcoma virus), HRAS (homologous to the oncogene from the Harvey rat sarcoma virus), and NRAS (first isolated from a human neuroblastoma). The different RAS genes are highly homologous but functionally distinct; the degree of redundancy remains a topic of investigation (reviewed in Pylayeva-Gupta et al. 2011). RAS proteins are small GTPases which cycle between inactive guanosine diphosphate (GDP)-bound and active guanosine triphosphate (GTP)-bound forms. RAS proteins are central mediators downstream of growth factor receptor signaling and therefore are critical for cell proliferation, survival, and differentiation. RAS can activate several downstream effectors, including the PI3K-AKT-mTOR pathway, which is involved in cell survival, and the RAS-RAF-MEK-ERK pathway, which is involved in cell proliferation.

RAS has been implicated in the pathogenesis of several cancers. Activating mutations within the RAS gene result in constitutive activation of the RAS GTPase, even in the absence of growth factor signaling. The result is a sustained proliferation signal within the cell. Specific RAS genes are recurrently mutated in different malignancies. NRAS mutations are particularly common in melanoma, hepatocellular carcinoma, myeloid leukemias, and thyroid carcinoma (for reviews see Karnoub and Weinberg 2008 and Schubbert, Shannon, and Bollag 2007).

Somatic mutations in NRAS have been found in ˜1% of all NSCLC (Brose et al. 2002; Ding et al. 2008; Ohashi et al. 2013). NRAS mutations are more commonly found in lung cancers with adenocarcinoma histology and in those with a history of smoking (Ohashi et al. 2013). In the majority of cases, these mutations are missense mutations that introduce an amino acid substitution at position 61. Mutations at position 12 have also been described (Ohashi et al. 2013). The result of these mutations is constitutive activation of NRAS signaling pathways.

In one aspect of the present invention, a method of treating a subject having a cancer with a NRAS gene mutation is provided wherein the subject is administered a CDK 4/6 inhibitor described herein in combination with dactolisib. In an alternative embodiment, provided is a method of treating a subject having a cancer with a NRAS gene mutation wherein the subject is administered a CDK 4/6 inhibitor described herein in combination with idelalisib, copanlisib, taselisib, perifosine, buparlisib, duvelisib, alpelisib, or umbralisib, or a combination thereof. In some embodiments, the NRAS mutation encodes for a Q61K substitution. In some embodiments, the NRAS mutation encodes for a Q61L, Q61R, or Q61H substitution. In some embodiments, the NRAS mutation encodes for a G12 C, G12R, G12S, G12A, or G12D substitution. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In some embodiments, the cancer having a NRAS gene mutation is selected from skin cutaneous melanoma, rectum adenocarcinoma, thyroid carcinoma, uterine corpus endometrial carcinoma, acute myeloid leukemia, colon adenocarcinoma, testicular germ cell tumors, thymoma, cholangiocarcinoma, bladder urothelial carcinoma, uterine carcinosarcoma, kidney chromophobe, cervical squamous cell carcinoma, endocervical adenocarcinoma, liver hepatocellular carcinoma, glioblastoma multiforme, lung squamous cell carcinoma, ovarian serous cystadenocarcinoma, adrenocortical carcinoma, stomach adenocarcinoma, and lung adenocarcinoma. In some embodiments, the NRAS cancer is melanoma, hepatocellular carcinoma, myeloid leukemias, NSCLC, or thyroid carcinoma. In some embodiments, the NRAS mutant cancer is NSCLC. In some embodiments, the cancer having a NRAS mutation encoding a Q61K substitution is skin cutaneous melanoma or rectum adenocarcinoma. In some embodiments, the cancer having a NRAS mutation encoding a Q61L substitution is skin cutaneous melanoma or glioblastoma multiforme. In some embodiments, the cancer having a NRAS mutation encoding a Q61R substitution is skin cutaneous melanoma, thyroid carcinoma, or cholangiocarcinoma. In some embodiments, the cancer having a NRAS mutation encoding a Q61H substitution is rectum adenocarcinoma or skin cutaneous melanoma. In some embodiments, the cancer having a NRAS mutation encoding a G12C substitution is rectum adenocarcinoma or colon adenocarcinoma. In some embodiments, the cancer having a NRAS mutation encoding a G12R substitution is skin cutaneous melanoma. In some embodiments, the cancer having a NRAS mutation encoding a G12A substitution is colon adenocarcinoma or skin cutaneous melanoma. In some embodiments, the cancer having a NRAS mutation encoding a G12S substitution is a testicular germ cell tumor or skin cutaneous melanoma. In some embodiments, the cancer having a NRAS mutation encoding a G12D substitution is rectum adenocarcinoma or acute myeloid leukemia.

In an alternative embodiment, the subject having a NRAS mutant cancer is administered a CDK 4/6 inhibitor described herein in combination with abemaciclib, abiraterone, afatinib, bosutinib, cabozantinib, dasatinib, enasidenib, enzalutamide, erlotinib, everolimus, erdafitinib, fulvestrant, gefitinib, ibrutinib, imatinib, ipatasertib, lapatinib, nilotinib, niraparib, olaparib, olarutumab, osimertinib, palbociclib, pazopanib, PF7775, Ponatinib, ramucirumab, regorafenib, ribociclib, rucaparib, savolitinib, sorafenib, sunitinib, talazoparib, trastuzumab, trilaciclib, or vistusertib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a NRAS mutant cancer is administered a CDK 4/6 inhibitor described herein in combination with alectinib, alpelisib, binimetinib, brigatinib, ceritinib, cobimetinib, copanlisib, crizotinib, dabrafenib, encorafenib, idelalisib, lorlatinib, SCH772984, selumetinib, trametinib, ulixertinib, or verumarenib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a NRAS mutant cancer is administered a CDK 4/6 inhibitor described herein in combination with a RET inhibitor. In some embodiments, the RET inhibitor is pralsetinib. In some embodiments, the RET inhibitor is agerafenib. In some embodiments, the RET inhibitor is vandentanib. In some embodiments, the RET inhibitor is lenvatinib. In some embodiments, the RET inhibitor is apatanib. In some embodiments, the RET inhibitor is sitravatinib. In some embodiments, the RET inhibitor is LOXO-292. In some embodiments, the CDK4/6 inhibitor administered is selected from Compound I, Compound IA, and Compound IA Form B. In some embodiments, the CDK4/6 inhibitor administered is Compound IA Form B.

### PIK3CA

Phosphatidyl 3-kinases (PI3K) are a family of lipid kinases involved in many cellular processes, including cell growth, proliferation, differentiation, motility, and survival. PI3K is a heterodimer composed of 2 subunits—an 85 kDa regulatory subunit (p85) and a 110 kDa catalytic subunit. The PIK3CA gene encodes p110a, one of the catalytic subunits.

PI3K converts PI(4,5)P2 [Phosphatidylinositol 4,5-bisphosphate] to PI(3,4,5)P3 [Phosphatidylinositol (3,4,5)-trisphosphate] on the inner leaflet of the cell membrane. PI(3,4,5)P3 recruits important downstream signaling proteins, such as AKT, to the cell membrane resulting in increased activity of these proteins.

Mutant PIK3CA has been implicated in the pathogenesis of several cancers, including colon cancer, gliomas, gastric cancer, breast cancer, endometrial cancer, and lung cancer (Samuels et al. 2004).

Somatic mutations in PIK3CA have been found in 1-3% of all NSCLC (COSMIC; Kawano et al. 2006; Samuels et al. 2004). These mutations usually occur within two “hotspot” areas within exon 9 (the helical domain) and exon 20 (the kinase domain). PIK3CA mutations appear to be more common in squamous cell histology compared to adenocarcinoma (Kawano et al. 2006) and occur in both never smokers and ever smokers. PIK3CA mutations can co-occur with EGFR mutations (Kawano et al. 2006; Sun et al. 2010). In addition, PIK3CA mutations have been detected in a small percentage (˜5%) of EGFR-mutated lung cancers with acquired resistance to EGFR TKI therapy (Sequist et al. 2011).

In some embodiments, provided is a method of treating a subject having a cancer with a PIK3CA gene mutation wherein the subject is administered a CDK 4/6 inhibitor described herein in combination with dabrafenib, selumetinib, ulixertinib, or dactolisib, or a combination thereof. In an alternative embodiment, provided is a method of treating a subject having a cancer with a PIK3CA gene mutation wherein the subject is administered a CDK 4/6 inhibitor described herein in combination with encorafenib, vemurafenib, idelalisib, copanlisib, taselisib, perifosine, buparlisib, duvelisib, alpelisib, SCH772984, trametinib, cobimetinib, binimetinib, or umbralisib, or a combination thereof. In some embodiments, the PIK3CA gene mutation encodes for an E542K substitution and the kinase inhibitor administered in combination with the CDK 4/6 inhibitor is dabrafenib, selumetinib, or ulixertinib, or a combination thereof. In some embodiments, the PIK3CA gene mutation encodes for a H1047R substitution and the kinase inhibitor administered in combination with the CDK 4/6 inhibitor is selumitinib or dactolisib. In some embodiments, the PIK3CA gene mutation encodes for a G106-R108 deletion and the kinase inhibitor administered in combination with the CDK 4/6 inhibitor is dactolisib. In some embodiments, the PIK3CA mutant cancer is also Rb-protein negative. In some embodiments, the PIK3CA gene mutation encodes for a E545Q or H1047L mutation. In some embodiments, the CDK4/6 inhibitor is administered to the subject after the subject's cancer has developed resistance to a previous kinase inhibitor therapy. In some embodiments, the CDK4/6 inhibitor administered is selected from Compound I, Compound IA, and Compound IA Form B. In some embodiments, the CDK4/6 inhibitor administered is Compound IA Form B.

In some embodiments, the PIK3CA mutant cancer is selected from uterine corpus endometrial carcinoma, uterine carcinosarcoma, breast invasive carcinoma, colon adenocarcinoma, cervical squamous cell carcinoma, endocervical adenocarcinoma, bladder urothelial carcinoma, head and neck squamous cell carcinoma, stomach adenocarcinoma, rectum adenocarcinoma, lung squamous cell carcinoma, glioblastoma multiforme, esophageal carcinoma, brain lower grade glioma, cholangiocarcinoma, lung adenocarcinoma, ovarian serous cystadenocarcinoma, liver hepatocellular carcinoma, sarcoma, skin cutaneous melanoma, pancreatic adenocarcinoma, prostate adenocarcinoma, and testicular germ cell tumors. In some embodiments, the PIK3CA mutant cancer is a colon cancer, glioma, gastric cancer, breast cancer, endometrial cancer, or lung cancer. In some embodiments, the cancer is NSCLC. In some embodiments, the cancer harboring a PI3KCA mutation encoding a E542G substitution is colon adenocarcinoma or breast invasive carcinoma. In some embodiments, the cancer harboring a PI3KCA mutation encoding a H1047R substitution is breast invasive carcinoma, uterine carcinosarcoma, or uterine corpus endometrial carcinoma. In some embodiments, the cancer harboring a PI3KCA mutation encoding a E545Q substitution is bladder urothelial carcinoma or lung squamous cell carcinoma. In some embodiments, the cancer harboring a PI3KCA mutation encoding a H1047L substitution is cholangiocarcinoma, esophageal carcinoma, or uterine corpus endometrial carcinoma.

In an alternative embodiment, the subject having a PI3KCA mutant cancer is administered a CDK 4/6 inhibitor described herein in combination with abemaciclib, abiraterone, afatinib, bosutinib, cabozantinib, dasatinib, enasidenib, enzalutamide, erlotinib, everolimus, erdafitinib, fulvestrant, gefitinib, ibrutinib, imatinib, ipatasertib, lapatinib, nilotinib, niraparib, olaparib, olarutumab, osimertinib, palbociclib, pazopanib, PF7775, Ponatinib, ramucirumab, regorafenib, ribociclib, rucaparib, savolitinib, sorafenib, sunitinib, talazoparib, trastuzumab, trilaciclib, or vistusertib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a PI3KCA mutant cancer is administered a CDK 4/6 inhibitor described herein in combination with alectinib, alpelisib, binimetinib, brigatinib, ceritinib, cobimetinib, copanlisib, crizotinib, dabrafenib, encorafenib, idelalisib, lorlatinib, SCH772984, selumetinib, trametinib, ulixertinib, or verumarenib, or combinations thereof. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject having a PI3KCA mutant cancer is administered a CDK 4/6 inhibitor described herein in combination with a RET inhibitor. In some embodiments, the RET inhibitor is pralsetinib. In some embodiments, the RET inhibitor is agerafenib. In some embodiments, the RET inhibitor is vandentanib. In some embodiments, the RET inhibitor is lenvatinib. In some embodiments, the RET inhibitor is apatanib. In some embodiments, the RET inhibitor is sitravatinib. In some embodiments, the RET inhibitor is LOXO-292. In some embodiments, the CDK4/6 inhibitor administered is selected from Compound I, Compound IA, and Compound IA Form B. In some embodiments, the CDK4/6 inhibitor administered is Compound IA Form B.

In an alternative embodiment, the subject has a cancer harboring a PIK3R1 mutation.

### Kinase Inhibitors

As contemplated herein, the present invention provides methods of treating a patient with a cancer having an identified driver mutation by administering a selective CDK4/6 inhibitor in combination or alternation with a kinase inhibitor as described herein. Kinase inhibitors for use in the invention include, but are not limited to, dabrafenib, sulemetinib, ulixertinib, dactolisib, crizotinib, alectinib, lapatinib, and trametinib which are described further below. In an alternative embodiment, kinase inhibitors for use in the invention include, but are not limited to, encorafenib, vemurafenib, idelalisib, copanlisib, taselisib, perifosine, buparlisib, duvelisib, alpelisib, umbralisib, ceritinib, brigatinib, and entrectinib, which are described further below. In an alternative embodiment, kinase inhibitors for use in the invention include RET inhibitors, including but not limited to agerafenib, pralsetinib, vandentanib, lenvatinib, apatanib, LOXO-292 and sitravatinib.

Dabrafenib (Tafinlar, GSK2118436) is a reversible ATP-competitive BRAF inhibitor used in the treatment of BRAF V600-mutated cancers and has the chemical structure:

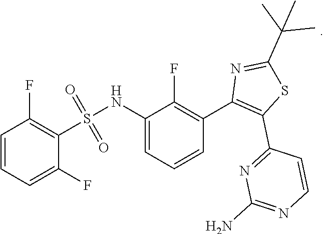 [SMILES: CC(C)(C)c1nc(-c2cccc(NS(=O)(=O)c3c(F)cccc3F)c2F)c(-c2ccnc(N)n2)s1]

Selumetinib (AZD6244) is a MEK1/MEK2 inhibitor under investigation for the treatment of cancers that contains BRAF mutations and has the chemical structure:

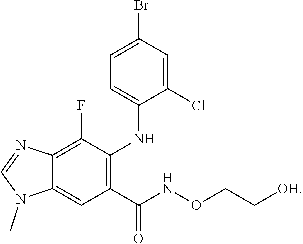 [SMILES: Cn1cnc2c(F)c(Nc3ccc(Br)cc3Cl)c(C(=O)NOCCO)cc21]

Ulixertinib (BVD-523) is a reversible, ATP-competitive ERK1/2 inhibitor with high potency and ERK1/2 selectivity. Ulixertinib has the chemical structure:

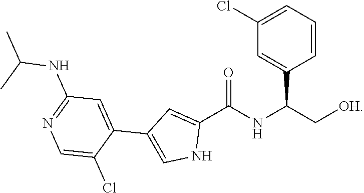 [SMILES: CC(C)Nc1cc(-c2c[nH]c(C(=O)N[C@H](CO)c3cccc(Cl)c3)c2)c(Cl)cn1]

Dactolisib (NVP-BEZ235) is a PI3K/mTOR inhibitor that has the chemical structure:

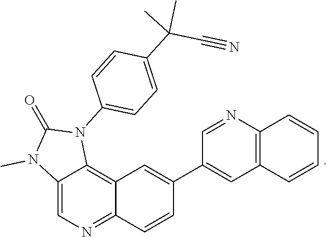 [SMILES: Cn1c(=O)n(-c2ccc(C(C)(C)C#N)cc2)c2c3cc(-c4cnc5ccccc5c4)ccc3ncc21]

Crizotinib (Xalkori) is a selective inhibitor of the kinase activity of the EML4-ALK fusion protein that drives malignant phenotypes in NSCLC. Crizotinib has the chemical structure:

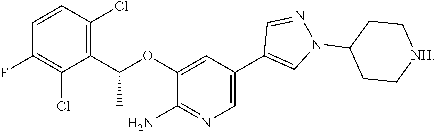 [SMILES: C[C@@H](Oc1cc(-c2cnn(C3CCNCC3)c2)cnc1N)c1c(Cl)ccc(F)c1Cl]

Alectinib (Alecensa) is a selective inhibitor of the kinase activity of the EML4-ALK fusion protein and has the chemical structure:

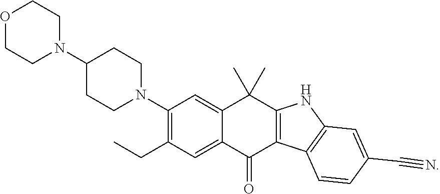 [SMILES: CCc1cc2c(cc1N1CCC(N3CCOCC3)CC1)C(C)(C)c1[nH]c3cc(C#N)ccc3c1C2=O]

Lapatinib (Tykerb or Tyverb) is a due HER2/neu and EGFR inhibitor that binds to the intracellular phosphorylation domain to prevent receptor autophosphorylation upon ligand binding. Lapatinib has the chemical structure:

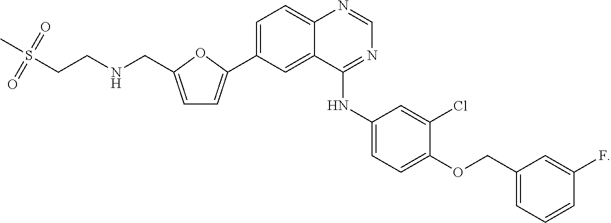 [SMILES: CS(=O)(=O)CCNCc1ccc(-c2ccc3ncnc(Nc4ccc(OCc5cccc(F)c5)c(Cl)c4)c3c2)o1]

Trametinib is a MEK1/MEK2 inhibitor used in the treatment of tumors carrying the BRAF V600E mutation that has the chemical structure:

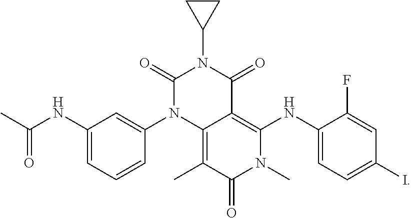 [SMILES: CC(=O)Nc1cccc(-n2c(=O)n(C3CC3)c(=O)c3c(Nc4ccc(I)cc4F)n(C)c(=O)c(C)c32)c1]

Encorafenib (LGX818) is BRAF inhibitor currently being investigated for the treatment of V600E mutant cancers that has the chemical structure:

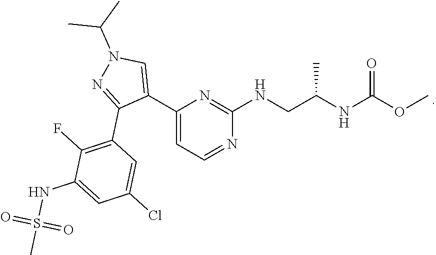 [SMILES: COC(=O)N[C@@H](C)CNc1nccc(-c2cn(C(C)C)nc2-c2cc(Cl)cc(NS(C)(=O)=O)c2F)n1]

Vemurafenib (Zelboraf) is a BRAF inhibitor used in the treatment of V600E mutant cancers that has the chemical structure:

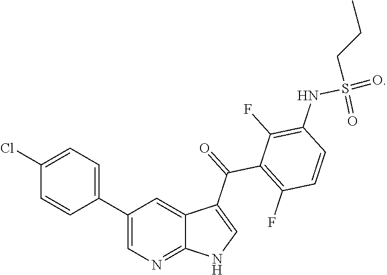 [SMILES: CCCS(=O)(=O)Nc1ccc(F)c(C(=O)c2c[nH]c3ncc(-c4ccc(Cl)cc4)cc23)c1F]

Idelalisib (Zydelig) is PI3K inhibitor using in the treatment of certain hematological malignancies that has the chemical structure:

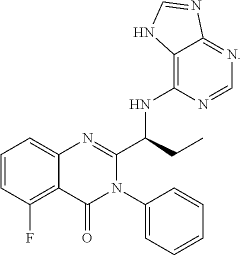 [SMILES: CC[C@H](Nc1ncnc2nc[nH]c12)c1nc2cccc(F)c2c(=O)n1-c1ccccc1]

Copanlisib (Aliqopa) is a PI3K inhibitor approved for the treatment of relapsed follicular lymphoma that has the chemical structure:

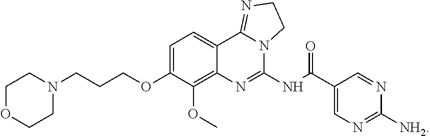 [SMILES: COc1c(OCCCN2CCOCC2)ccc2c1N=C(NC(=O)c1cnc(N)nc1)N1CCN=C21]

Taselisib (GDC-0032) is an experimental PI3K inhibitor in development for the treatment of metastatic breast cancer and NSCLC that has the chemical structure:

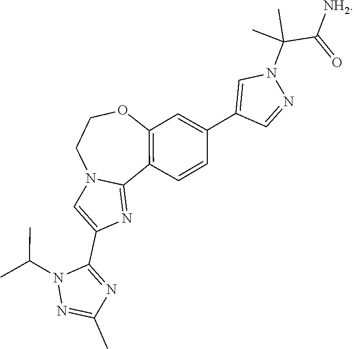

Perifosine (KRX-0401) is an alkyl-phospholipid in development as a PI3K inhibitor that has the chemical structure:

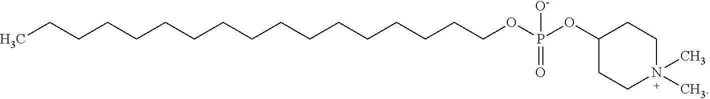

Buparlisib (BKM120) is an investigation PI3K inhibitor in clinical trials for the treatment of advanced HR+/HER2 endocrine-resistant breast cancer that has the chemical structure:

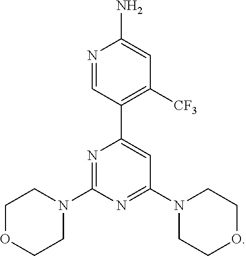 [SMILES: Nc1cc(C(F)(F)F)c(-c2cc(N3CCOCC3)nc(N3CCOCC3)n2)cn1]

Duvelisib (IPI-145) is a PI3K inhibitor in clinical development for the treatment of hematological malignancies that has the chemical structure:

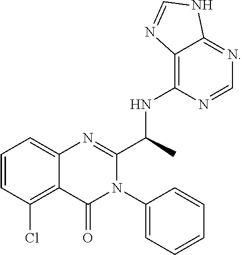 [SMILES: C[C@H](Nc1ncnc2[nH]cnc12)c1nc2cccc(Cl)c2c(=O)n1-c1ccccc1]

Alpelisib (BYL719) is a PI3K inhibitor in development for the treatment of several cancers that has the chemical structure:

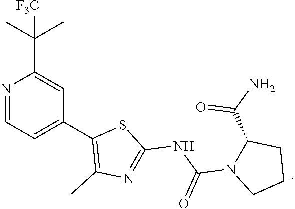 [SMILES: Cc1nc(NC(=O)N2CCC[C@H]2C(N)=O)sc1-c1ccnc(C(C)(C)C(F)(F)F)c1]

Umbralisib (TGR-1202_ is a PI3K inhibitor in development for the treatment of hematological malignancies that has the chemical structure:

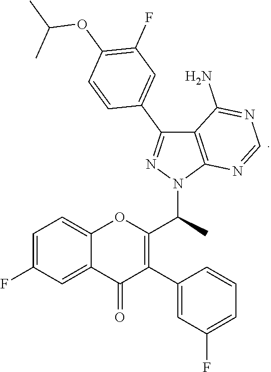 [SMILES: CC(C)Oc1ccc(-c2nn([C@@H](C)c3oc4ccc(F)cc4c(=O)c3-c3cccc(F)c3)c3ncnc(N)c23)cc1F]

Ceritinib (Zykania) is a ALK-positive inhibitor that is approved for the treatment of NSCLC that has the chemical structure:

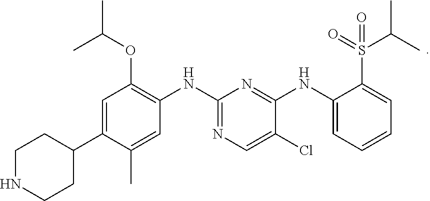 [SMILES: Cc1cc(Nc2ncc(Cl)c(Nc3ccccc3S(=O)(=O)C(C)C)n2)c(OC(C)C)cc1C1CCNCC1]

Brigatinib (Alunbrig) is an investigation dual ALK an EGFR inhibitor that has the chemical structure:

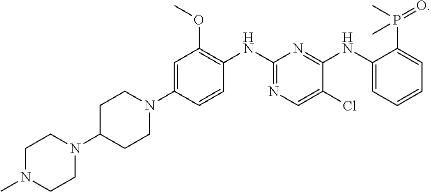 [SMILES: COc1cc(N2CCC(N3CCN(C)CC3)CC2)ccc1Nc1ncc(Cl)c(Nc2ccccc2P(C)(C)=O)n1]

Entrectinib (RXD-101 and NMS-E628) is an investigational inhibitor of Trk, ROS1, and ALK that has the chemical structure:

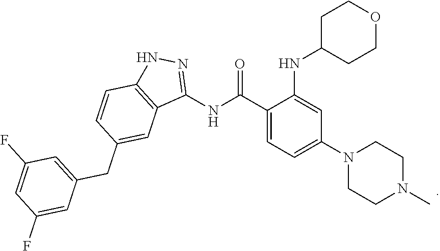 [SMILES: CN1CCN(c2ccc(C(=O)Nc3n[nH]c4ccc(Cc5cc(F)cc(F)c5)cc34)c(NC3CCOCC3)c2)CC1]

Trametinib (Mekinist) is a MEK inhibitor used in the treatment of metastatic melanoma carrying the BRAF V600E mutation that has the chemical structure:

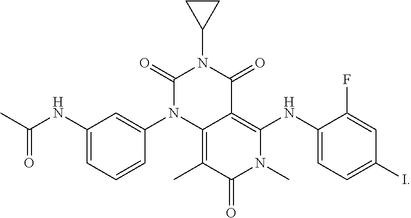 [SMILES: CC(=O)Nc1cccc(-n2c(=O)n(C3CC3)c(=O)c3c(Nc4ccc(I)cc4F)n(C)c(=O)c(C)c32)c1]

Cobimetinib (Cotellic) is a MEK inhibitor used to treat melanoma carrying the BRAF V600E mutation that has the chemical structure:

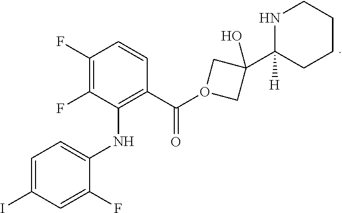

Binimetinib (MEK162 or ARRY-162) is a MEK inhibitor currently being developed to treat BRAF mutant melanoma that has the chemical structure:

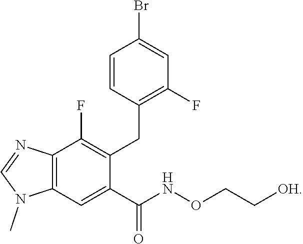 [SMILES: Cn1cnc2c(F)c(Cc3ccc(Br)cc3F)c(C(=O)NOCCO)cc21]

Lorlatinib (PF-6463922) is an experimental ROS1 and ALK inhibitor in clinical development for the treatment of NSCLC that has the chemical structure:

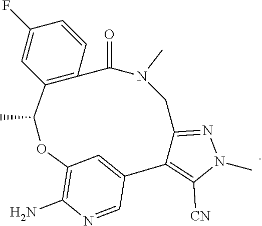

SCH772984 is a ERK1/2 inhibitor in development for the treatment of RAS or BRAF mutant cancer cells that has the chemical structure:

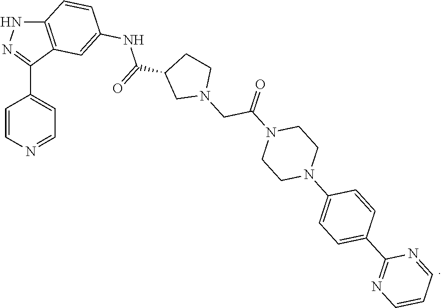 [SMILES: O=C(Nc1ccc2[nH]nc(-c3ccncc3)c2c1)[C@@H]1CCN(CC(=O)N2CCN(c3ccc(-c4ncccn4)cc3)CC2)C1]

Agerafenib (RXDX-105) is an orally available RET inhibitor having the chemical structure:

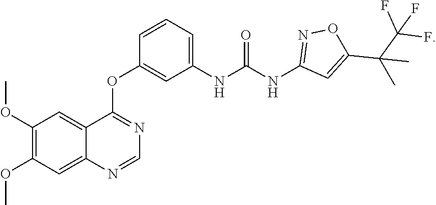 [SMILES: COc1cc2ncnc(Oc3cccc(NC(=O)Nc4cc(C(C)(C)C(F)(F)F)on4)c3)c2cc1OC]

Pralsetinib (BLU-667) is a highly potent, selective RET inhibitor having the chemical structure:

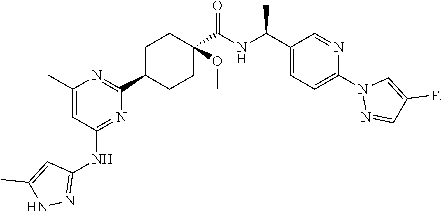 [SMILES: CO[C@]1(C(=O)N[C@@H](C)c2ccc(-n3cc(F)cn3)nc2)CC[C@H](c2nc(C)cc(Nc3cc(C)[nH]n3)n2)CC1]

Vandentanib is multiple kinase inhibitor with inhibitory activity against RET having the chemical structure:

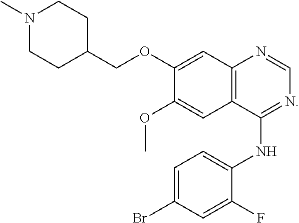 [SMILES: COc1cc2c(Nc3ccc(Br)cc3F)ncnc2cc1OCC1CCN(C)CC1]

Lenvatinib is a multiple kinase inhibitor with inhibitory activity against RET having the chemical structure:

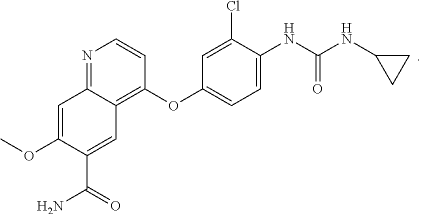 [SMILES: COc1cc2nccc(Oc3ccc(NC(=O)NC4CC4)c(Cl)c3)c2cc1C(N)=O]

Apatanib is a VEGFR2 inhibitor that also shows inhibitory activity against RET having the chemical structure:

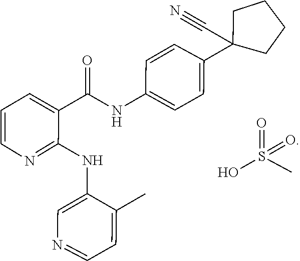

LOXO-292 is a selective RET inhibitor having the chemical structure:

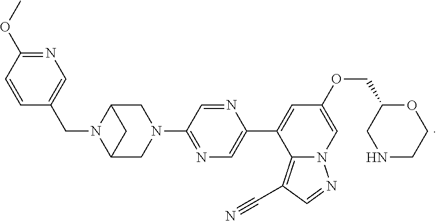 [SMILES: COc1ccc(CN2C3CC2CN(c2cnc(-c4cc(OC[C@H]5CNCCO5)cn5ncc(C#N)c45)cn2)C3)cn1]

Sitravatinib is a multiple kinase inhibitor with inhibitory activity of RET having the chemical structure:

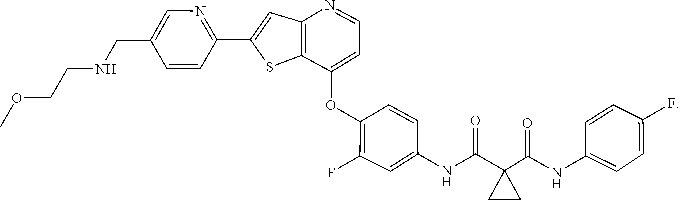 [SMILES: COCCNCc1ccc(-c2cc3nccc(Oc4ccc(NC(=O)C5(C(=O)Nc6ccc(F)cc6)CC5)cc4F)c3s2)nc1]

### Pharmaceutical Compositions and Dosage Forms

In other aspects, this invention is a pharmaceutical composition comprising a therapeutically effective amount of a selective CDK4/6 inhibitor selected from Compound I, Compound IA, Compound IA Form B, Compound II, Compound III, and Compound IV and an additional kinase inhibitor described herein, and one or more pharmaceutically acceptable diluents, preservatives, solubilizers, emulsifiers, adjuvants, excipients, or carriers. Such excipients include liquids such as water, saline, glycerol, polyethylene glycol, hyaluronic acid, ethanol, and the like.

The term “pharmaceutically acceptable carrier” refers to a diluent, adjuvant, excipient or carrier with which a compound of the disclosure is administered. The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. An appropriate “effective” amount in any individual case can be determined by one of ordinary skill in the art using routine experimentation. “Pharmaceutically acceptable carriers” for therapeutic use are well known in the pharmaceutical art, and are described, for example, in Remington's Pharmaceutical Sciences, 18th Edition (Easton, Pa.: Mack Publishing Company, 1990). For example, sterile saline and phosphate-buffered saline at physiological pH can be used. Preservatives, stabilizers, dyes and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id.

Suitable excipients for non-liquid formulations are also known to those of skill in the art. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

In general, the compositions of the disclosure will be administered in a therapeutically effective amount by any of the accepted modes of administration. Suitable dosage ranges depend upon numerous factors such as the severity of the disease to be treated, the age and relative health of the patient, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

Thus, the compositions of the disclosure can be administered as pharmaceutical formulations including those suitable for oral (including buccal and sub-lingual), rectal, nasal, topical, pulmonary, vaginal or parenteral (including intramuscular, intra-arterial, intrathecal, subcutaneous and intravenous) administration or in a form suitable for administration by inhalation or insufflation. The preferred manner of administration is intravenous or oral using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another embodiment is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in an acceptably nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacterium retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

Preferred formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and non-sensitizing.

Formulations for buccal administration include tablets, lozenges, gels and the like. Alternatively, buccal administration can be effected using a transmucosal delivery system as known to those skilled in the art. The compounds of the disclosure can also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In some embodiments, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compositions of the disclosure can be formulated for aerosol administration, particularly to the respiratory tract and including intranasal administration. The compound may, for example generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve. Alternatively the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

A pharmaceutically or therapeutically effective amount of the composition will be delivered to the patient. The precise effective amount will vary from patient to patient and will depend upon the species, age, the patient's size and health, the nature and extent of the condition being treated, recommendations of the treating physician, and the therapeutics or combination of therapeutics selected for administration. the effective amount for a given situation can be determined by routine experimentation. For purposes of the disclosure, a therapeutic amount may for example be in the range of about 0.01 mg/kg to about 250 mg/kg body weight, more preferably about 0.1 mg/kg to about 10 mg/kg, in at least one dose. In larger mammals, the indicated daily dosage can be from about 1 mg to 1500 mg, one or more times per day, more preferably in the range of about 10 mg to 600 mg. The patient can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

The therapeutically effective dosage of any active compound described herein will be determined by the health care practitioner depending on the condition, size and age of the patient as well as the route of delivery. In one non-limited embodiment, a dosage from about 0.1 to about 200 mg/kg has therapeutic efficacy, with all weights being calculated based upon the weight of the active compound, including the cases where a salt is employed. In some embodiments, the dosage may be the amount of compound needed to provide a serum concentration of the active compound of up to about 10 nM, 50 nM, 100 nM, 200 nM, 300 nM, 400 nM, 500 nM, 600 nM, 700 nM, 800 nM, 900 nM, 1 μM, 5 μM, 10 μM, 20 μM, 30 μM, or 40 μM.

In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form. Examples of dosage forms with at least 5, 10, 15, 20, 25, 50, 100, 200, 250, 300, 400, 500, 600, 700, or 750 mg of active compound, or its salt. The pharmaceutical composition may also include a molar ratio of the active compound and an additional active agent, in a ratio that achieves the desired results.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### EXAMPLES

### Example 1. A NSCLC PDX Model Panel Demonstrated Varying Sensitivity to Treatment with Compound 1

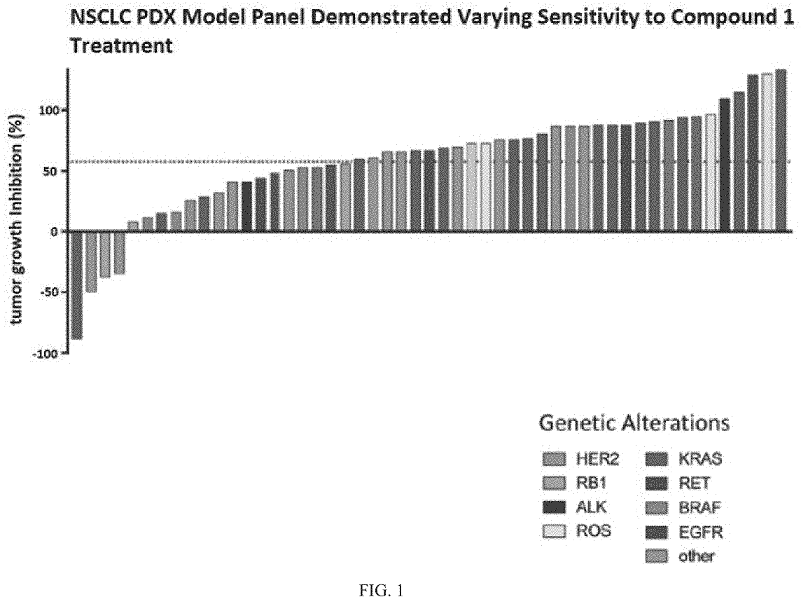

NSCLC PDX models (n=60) were treated for up to 28 days with daily oral doses of vehicle or Compound 1 (100 mg/kg). Tumor growth inhibition (TGI) was calculated when tumors reached pre-specified tumor burden or on day 28. Genetic alterations from pre-treated samples were evaluated using FoundationOne. 58% TGI was used as the responder/non-responder cutoff for correlation analysis. As shown in FIG. 1, there was a range of efficacy associated with Compound 1 in the panel of NSCLC PDX models. Certain genetic alterations, such as KRAS and EGFR, demonstrated increased sensitivity while others, such as RB1, demonstrated resistance. The median TGI for adenocarcinoma was 73%, and the median TGI for squamous cell carcinoma was 66%.

### Example 2. Compound 1 Enhances the Anti-Proliferative Effect of Inhibitors Targeting Specific Oncogenic Drivers In Vitro

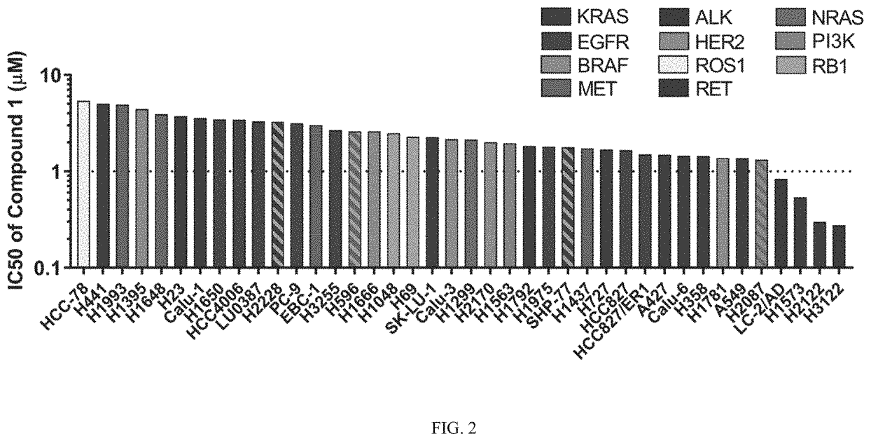

Lung cancer cell lines (n=40) harboring known oncogenic mutations were screen for sensitivity to Compound 1 alone or in combination with relevant targeted kinase inhibitors (Crown Bioscience, Taicang, China). As shown in FIG. 2, absolute IC₅₀ values from single-agent treatments with Compound 1 were calculated using a 2× doubling time cell proliferation assay (minimum 3 days) and were used to guide the design of the combination treatment assay. Growth inhibitor values were used to calculate the synergy scores of Compound 1 in combination with dabrafenib, selumetinib, ulixertinib, dactolisib, osimertinib, crizotinib, alectinib, or lapatinib using the Loewe Additivity model. For the drug synergy screen of NSCLC cell lines, 9×9 combination matrices nominally centered around single-agent IC₅₀ values were created, and cell proliferation for each condition was measured and compared to vehicle control after two population doubling times. Based on this data, average Loewe synergy scores for each drug combination were calculated using exclusion criteria that omitted from the calculation those conditions which produced <25% inhibition in combination or conditions for which either associated single-agent concentration produced >90% inhibition. Loewe synergy scores >5 are indicative of synergy, and scores <−5 represent antagonism.

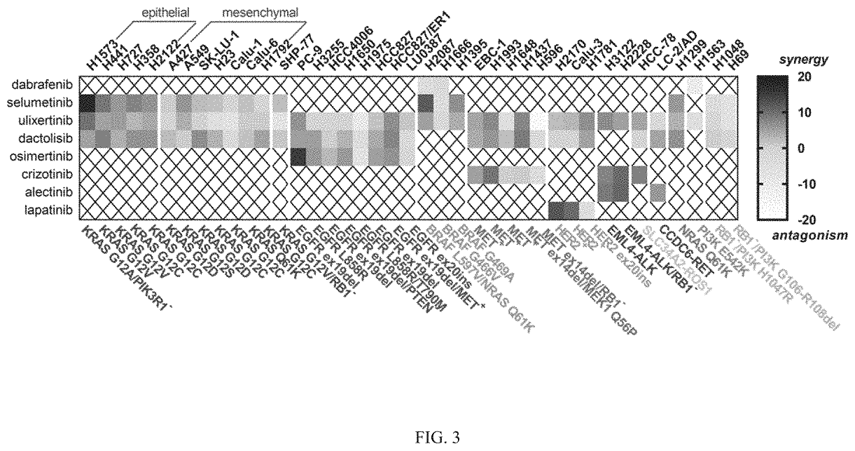

As demonstrated in FIG. 3, significant synergy was observed between Compound 1 and kinase inhibitors harboring EGFR mutations, ALK fusions, HER2 amplification, and MET exon 14 deletions. Specifically, Compound 1 frequently synergized with osimertinib (EGFR inhibitor) or dactolisib (PI3K/mTOR inhibitor) in EGFR-mutant NSCLCs, even in the context of MET amplification (a common mechanism of resistance to EGFR inhibitors in NSCLC). The highest degree of synergy was seen with Compound 1 and lapatinib (HER2 inhibitor) in HER2-amplified NSLC cells, with Loewe synergy scores >10. In a HER2-mutated NSCLC cell line resistant to lapatinib inhibition (H1781), Compound 1 was synergistic with ulixertinib (ERK inhibitor). NSCLC cells with ALK rearrangements also demonstrated a high degree of synergy between Compound 1 and alectinib (ALK inhibitor) or crizotinib (ALK/MET/ROS1 inhibitor), with this synergy even seen in an RB1 negative cell line (H2228). In NSCLC cells harboring MET exon 14 deletions, Compound 1 synergized with crizotinib (ALK/MET/ROS1 inhibitor) and ulixertinib (ERK inhibitor). Epithelial KRAS-mutant cell line (H2122) yielded a highly synergistic Loewe synergy score of 11.1 for a combination of Compound 1 and dactolisib (PI3K/mTOR inhibitor).

### Example 3. Combination Treatment with Compound 1 Augments the Anti-Proliferative and Apoptotic Signaling Pathways

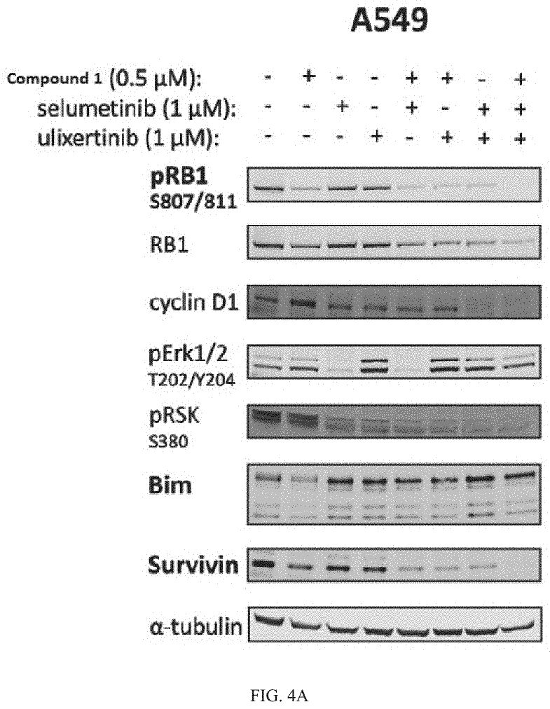

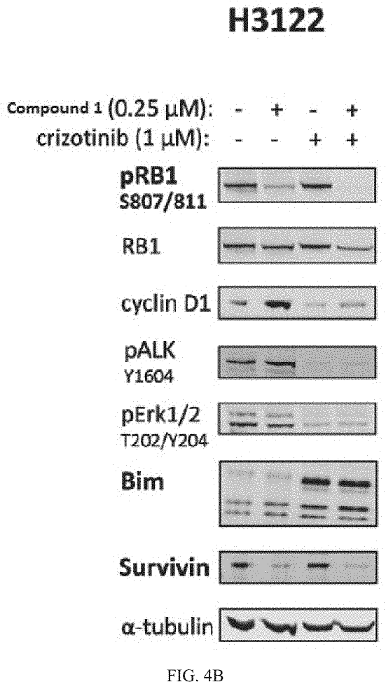

A549 (KRAS^{G12S} and CDKN2A null) NSCLC cells were treated with Compound 1 (0.5 selumetinib (1 and/or ulixertinib (1 μM) for 48 hours. Additionally, H3122 (EML4-ALK fusion) NSCLC cells were treated with Compound 1 (0.5 μM) and/or crizotinib (1 μM) for 48 hours. All cells were subjected to immunoblotting with a-tubulin used as the loading control. As shown in the immunoblots in FIGS. 4A and 4B, the enhanced efficacy of treatment combinations with Compound 1 may be due to profound suppression of RB phosphorylation coupled with an enhancement of a pro-apoptotic phenotype when compared to either single agent treatment.

### Example 4. Compound 1 Enhances the Efficacy of Selumetinib and Ulixertinib Therapy in a KRAS^{G12S} NSCLC Mouse Model

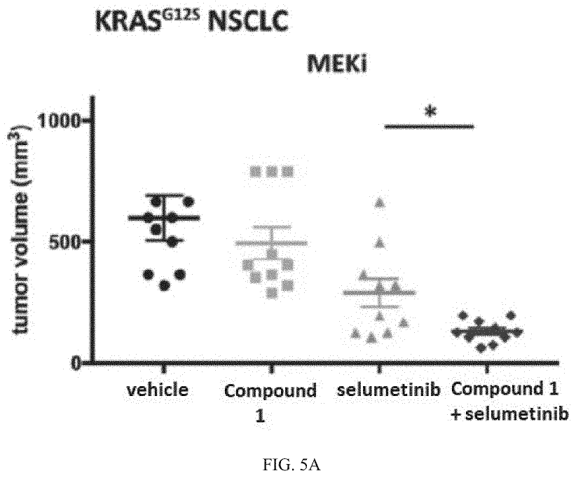

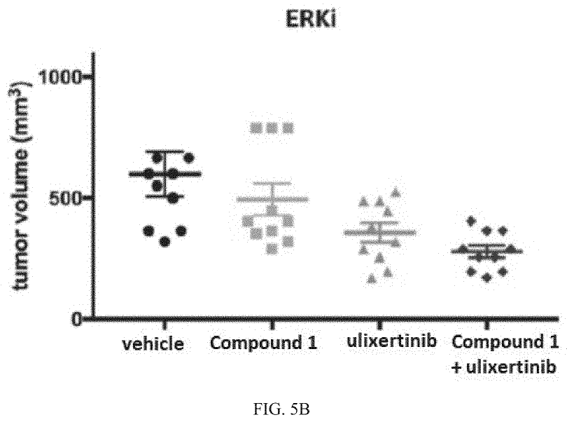

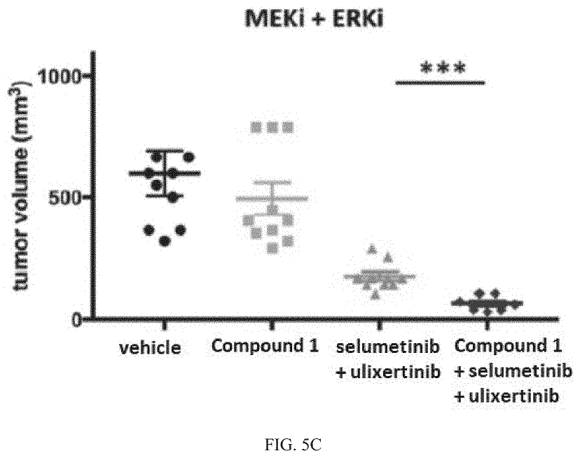

A549, a KRAS^{G12S} human NSCLC xenograft model, was treated with Compound 1 and/or ERKi/MEKi in vivo (Charles River Laboratories, Research Triangle Park, N.C.). Mice were given once daily doses of vehicle, Compound 1 (100 mg/kg), selumetinib (50 mg/kg), or twice daily doses of ulixertinib (50 mg/kg) alone or in combination by oral gaved for 30 days (n=7-10). As shown in FIGS. 5A, 5B, and 5C, significant decreases in tumor volume were observed with Compound 1 in combination with selumetinib and in combination with both selumetinib and ulixertinib.

### Example 5. Compound 1 Enhances the Efficacy of Crizotinib Therapy in an EML4-ALK NSCLC Mouse Model

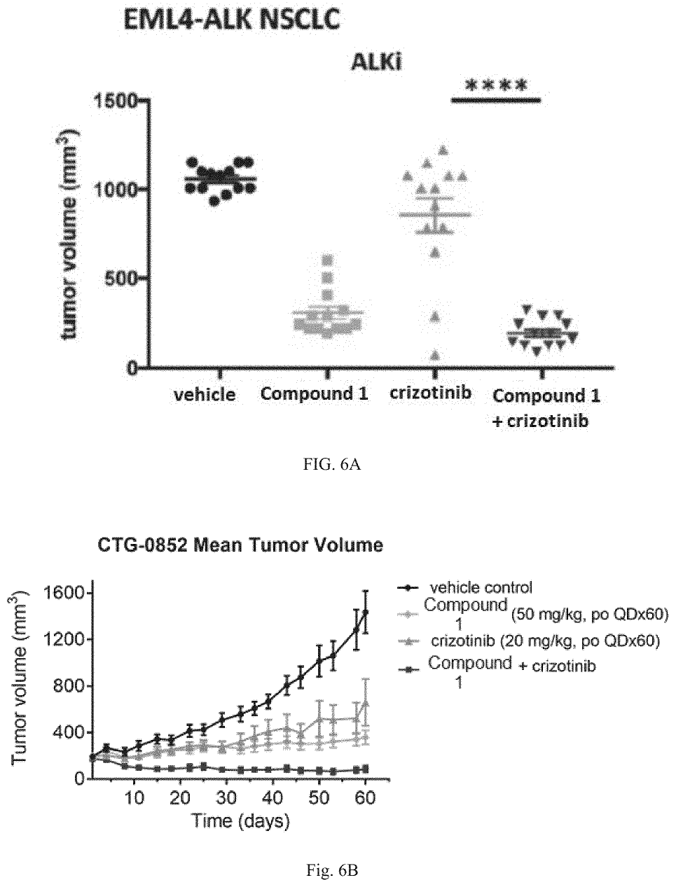

H3122, an EML4-ALK fusion human NSCLC xenograft model, was treated with Compound 1 and/or ALK treatment in vivo (MI Bioresearch, Ann Arbor, Mich.). Mice were given once daily doses of vehicle, Compound 1 (100 mg/kg), or crizotinib (25 mg/kg for the first 12 days, then 50 mg/kg) alone or in combination by oral gavage for 28 days (n=13). As shown in FIG. 6A, significant decreases in tumor volume were observed with Compound 1 in combination with crizotinib.

Mice bearing EML4-ALK NSCLC PDX tumors (Champions PDX model CTG-0852) were treated orally once daily with Compound I (50 mg/kg), crizotinib (20 mg/kg), or the combination for 60 days. As single-agents, Compound I and crizotinib promoted 87% and 63% TGI, respectively, after 60 days. As shown in FIG. 6B, combination treatment with compound I+crizotinib resulted in significant enhancement of antitumor efficacy (50% tumor regression from baseline after 60 days). The combination treatment was well-tolerated over the extended dosing period (60 days).

### Example 6. Compound 1 Enhances Efficacy of RET Inhibitors in RET-Rearranged Non-Small Cell Lung Cancer Cells

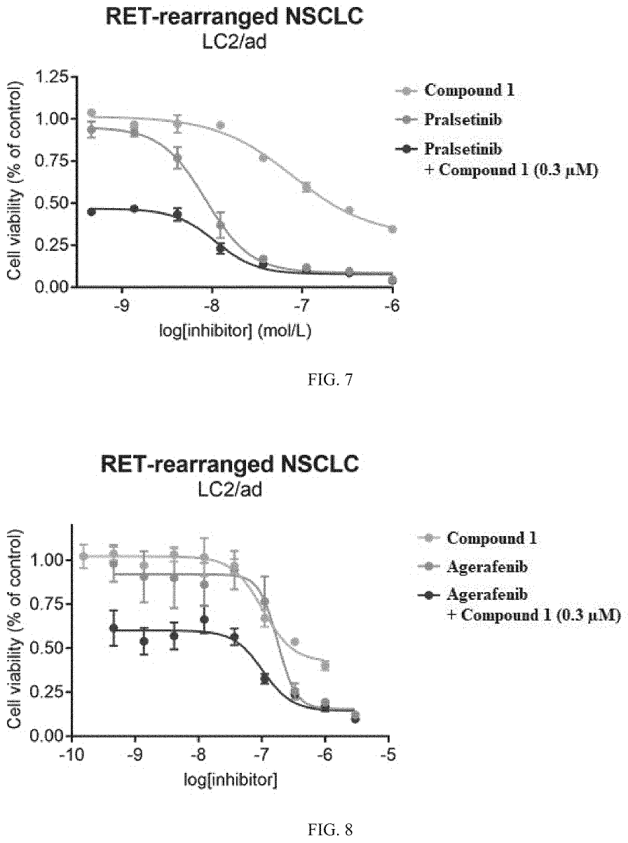

LC2/ad non-small cell lung cancer cells expressing an oncogenic CCDC6-RET fusion were dosed with increasing concentrations of Compound 1, a RET inhibitor selected from pralsetinib (BLU-667) or agerafenib (RXDX-105), or RET inhibitor+300 nM Compound 1. Cell proliferation was measured by CellTiter-Glo after six days. As shown in FIG. 7 for pralsetinib and in FIG. 8 for agerafenib, the combination of Compound 1+RET inhibitor significantly enhanced the antiproliferative effect compared to RET inhibitor alone.

### Example 7. Compound 1 Delays Resistance to RET Inhibitor in RET-Rearranged Non-Small Cell Lung Cancer Cells

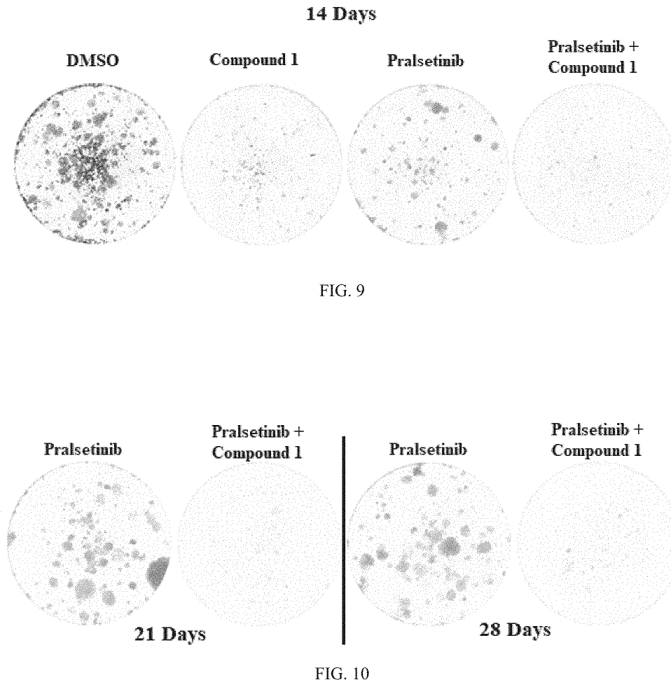

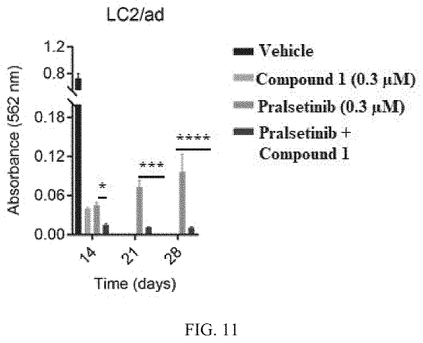

LC2/ad non-small cell lung cancer cells expressing an oncogenic CCDC6-RET fusion were seeded at low density in 6-well plates and treated with DMSO, 300 nM of the RET inhibitor pralsetinib (>10 fold above the IC₅₀ for cellular proliferation), 300 nM Compound 1, or the combination of pralsetinib and Compound 1. Each condition was repeated in triplicate in each 6-well plate. Inhibitor-containing media was refreshed every seven days. After 14 days the DMSO plate reached confluence, and on that day and every 7 days thereafter, 6-well plates were harvested, stained with crystal violet, and cell confluence was imaged (see FIGS. 9 and 10). The stain was then solubilized and quantified by measuring absorbance intensity at 562 nm on an absorbance plate reader (see FIG. 11). Combination treatment with Compound 1 and pralsetinib was found to significantly delay the outgrowth of resistant cell colonies compared to pralsetinib alone.

### Example 8. Conversion of Compound 1 to its HCl Counterpart, Compound 2

A representative synthesis of Compound IA is provided in Scheme 1.

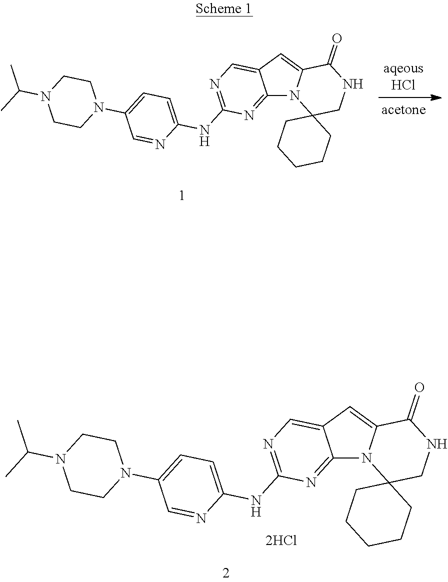 [SMILES: CC(C)N1CCN(c2ccc(Nc3ncc4cc5n(c4n3)C3(CCCCC3)CNC5=O)nc2)CC1 ; CC(C)N1CCN(c2ccc(Nc3ncc4cc5n(c4n3)C3(CCCCC3)CNC5=O)nc2)CC1]

Compound I (0.9 kg. 1.9 moles, 1 eq) was charged to a 22 L flask and dissolved in aqueous, 2 M hydrochloric acid solution (3.78 L). The solution was heated to 50±5° C., stirred for 30 minutes, and the resulting mixture filtered over Celite (alternatively the solution may be filtered through a 0.45 micron in-line filter) to afford Compound IA. The flask was rinsed with 0.1 M hydrochloric acid solution to collect any additional Compound IA. Compound IA was then heated to 50±5° C. while acetone (6.44 L) was slowly added. The solution was stirred at 50±5° C. for 30 minutes, the temperature was decreased to 20±5° C., and stirring continued for 2 hours. The solids were collected by filtration, washed with acetone, and dried to afford 820.90 g of Compound IA (82.1% yield). In one embodiment instead of acetone, ethanol is used.

### Example 9. Morphic Forms of Compound IA

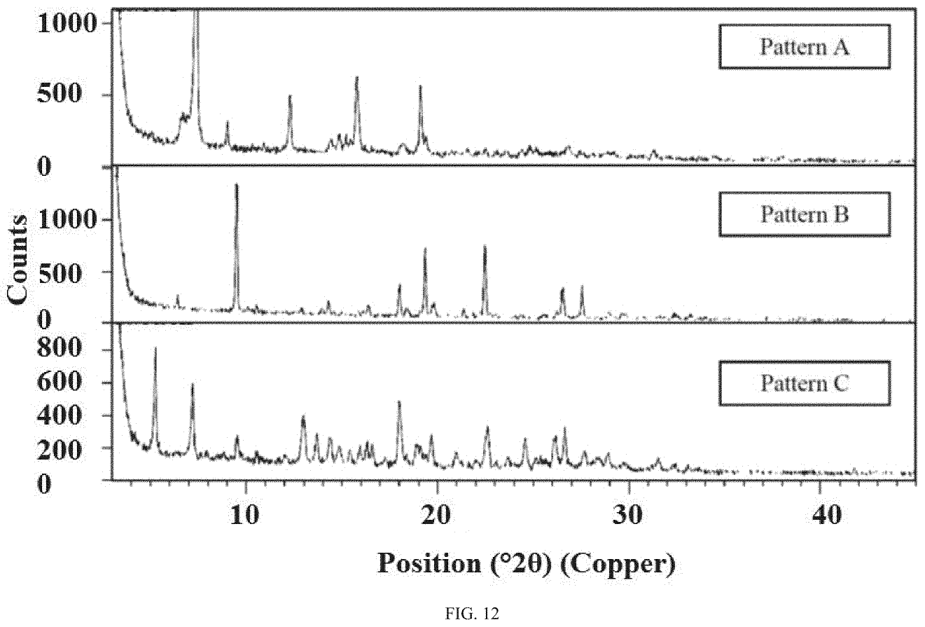

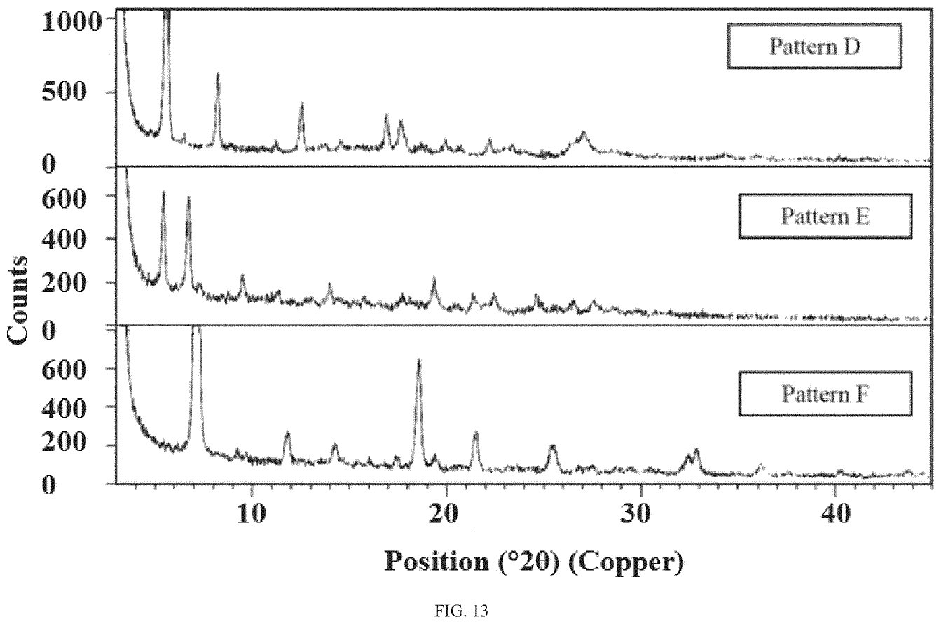

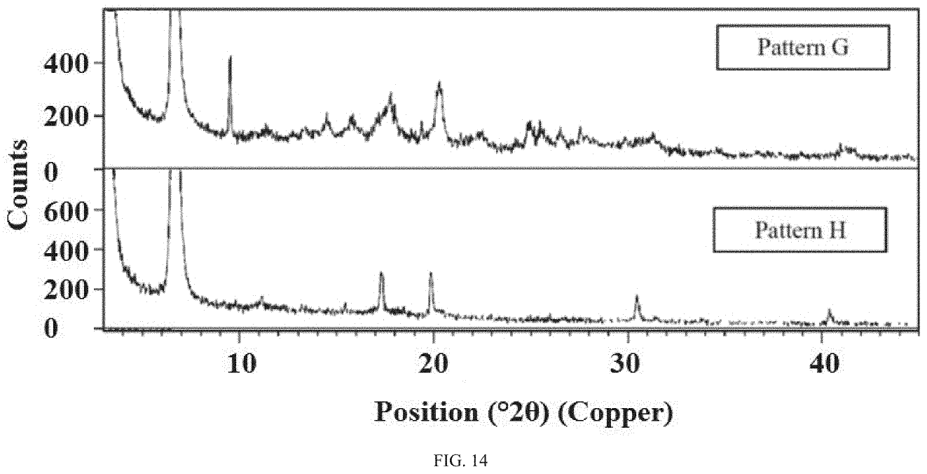

Eleven unique XRPD patterns (Form A-Form K) of Compound IA were obtained from crystallization and slurry experiments using various solvents. The conditions and XRPD results for these crystallization experiments are given in Tables 1-4. Single solvent crystallizations (Table 1) resulted in weak crystalline forms or Form A. Binary solvent crystallizations using water (Table 2) and MeOH (Table 3) as the primary solvent resulted in weak crystalline forms and Form A, Form B, Form F, Form G, and Form H. Solids recovered from slurry experiments after one and seven days of equilibration (Table 4) were analyzed by XRPD to determine the crystalline form, and after seven days, Form A, Form B, Form C, Form D, and Form E were observed. FIG. 12 shows the XRPD patterns of Form A, Form B, and Form C. FIG. 13 shows the XRPD patterns of Form D, Form E, and Form F. FIG. 14 shows the XRPD patterns of Form G and Form H.

TABLE 1
Single Solvent Crystallization Conditions and Results

| | Volume | Temp. | | Precipitation/ | |
|---|---|---|---|---|---|
| Solvent | (mL) | (° C.) | Cooling | Isolation | XRPD |
| Water | 2.0 | 60 | Slow (20° C./hr) | Turbid/Evap. | Weak crystalline |
| MeOH | 0.5 | 60 | Slow (20° C./hr) | ppt/filter | A |
| EtOH | 4.0 | 60 | Slow (20° C./hr) | ppt/filter | A |
| 1-PrOH | 4.0 | 60 | Slow (20° C./hr) | ppt/filter | Weak crystalline |
| 1-BuOH | 4.0 | 60 | Slow (20° C./hr) | ppt/filter | A |
| Water | 2.0 | 60 | Fast Cooling (4° C.) | Turbid/Evap. | Weak crystalline |
| MeOH | 0.5 | 60 | Fast Cooling (4° C.) | ppt/filter | Weak crystalline |
| EtOH | 4.0 | 60 | Fast Cooling (4° C.) | ppt/filter | A |
| 1-PrOH | 4.0 | 60 | Fast Cooling (4° C.) | ppt/filter | Weak crystalline |
| 1-BuOH | 4.0 | 60 | Fast Cooling (4° C.) | ppt/filter | Weak crystalline |

TABLE 2
Binary Solvent Crystallizations using water as Primary Solvent

| Primary | | | Anti | | |
|---|---|---|---|---|---|
| Solvent/Vol. | Temp. | | Solvent/Vol. | Precipitation/ | |
| (mL) | (° C.) | Cooling | (mL) | Isolation | XRPD |
| Water/0.5 | 60.0 | Fast Cooling (4° C.) | EtOH/5.0 | Clear/Evap. | Weak crystalline |
| Water/0.5 | 60.0 | Fast Cooling (4° C.) | n-PrOH/5.0 | Clear/Evap. | Weak crystalline |
| Water/0.5 | 60.0 | Fast Cooling (4° C.) | IPA/5.0 | ppt/filter | G |
| Water/0.5 | 60.0 | Fast Cooling (4° C.) | MeCN/5.0 | ppt/filter | Weak crystalline |
| Water/0.5 | 60.0 | Fast Cooling (4° C.) | THF/3.0 | ppt/filter | Weak crystalline |
| Water/0.5 | 60.0 | Fast Cooling (4° C.) | Acetone/3.5 | ppt/filter | G |
| Water/0.5 | 60.0 | Slow Cooling (20° C./hr) | EtOH/5.0 | Clear/Evap. | Weak crystalline |
| Water/0.5 | 60.0 | Slow Cooling (20° C./hr) | n-PrOH/5.0 | Clear/Evap. | H |
| Water/0.5 | 60.0 | Slow Cooling (20° C./hr) | IPA/5.0 | ppt/filter | B |
| Water/0.5 | 60.0 | Slow Cooling (20° C./hr) | MeCN/5.0 | ppt/filter | A |
| Water/0.5 | 60.0 | Slow Cooling (20° C./hr) | THF/3.0 | ppt/filter | G |
| Water/0.5 | 60.0 | Slow Cooling (20° C./hr) | Acetone/3.5 | ppt/filter | B |

TABLE 3
Binary Solvent Crystallizations using MeOH as Primary Solvent

| Primary | | | Anti | | |
|---|---|---|---|---|---|
| Solvent/Vol. | Temp. | | Solvent/Vol. | Precipitation/ | |
| (mL) | (° C.) | Cooling | (mL) | Isolation | XRPD |
| MeOH/0.5 | 60.0 | Fast Cooling (4° C.) | EtOH/5.0 | ppt/filter | A |
| MeOH/0.5 | 60.0 | Fast Cooling (4° C.) | n-PrOH/5.0 | ppt/filter | Weak crystalline |
| MeOH/0.5 | 60.0 | Fast Cooling (4° C.) | IPA/2.5 | ppt/filter | F |
| MeOH/0.5 | 60.0 | Fast Cooling (4° C.) | n-BuOH/5.0 | ppt/filter | Weak crystalline |
| MeOH/0.5 | 60.0 | Fast Cooling (4° C.) | MeCN/2.5 | ppt/filter | A |
| MeOH/0.5 | 60.0 | Fast Cooling (4° C.) | THF/0.5 | ppt/filter | A |
| MeOH/0.5 | 60.0 | Fast Cooling (4° C.) | 2-MeTHF/0.1 | ppt/filter | A |
| MeOH/0.5 | 60.0 | Fast Cooling (4° C.) | EtOAc/0.2 | ppt/filter | Weak crystalline |
| MeOH/0.5 | 60.0 | Fast Cooling (4° C.) | IPAc/0.1 | ppt/filter | A |
| MeOH/0.5 | 60.0 | Fast Cooling (4° C.) | Acetone/0.5 | ppt/filter | A |
| MeOH/0.5 | 60.0 | Slow Cooling (20° C./hr) | MEK/0.2 | ppt/filter | A |
| MeOH/0.5 | 60.0 | Slow Cooling (20° C./hr) | MIBK/0.1 | ppt/filter | Weak crystalline |
| MeOH/0.5 | 60.0 | Slow Cooling (20° C./hr) | DCM/5.0 | Clear/Evap. | A |
| MeOH/0.5 | 60.0 | Slow Cooling (20° C./hr) | Toluene/1.5 | ppt/filter | A |
| MeOH/0.5 | 60.0 | Slow Cooling (20° C./hr) | MTBE/0.1 | ppt/filter | A |
| MeOH/0.5 | 60.0 | Slow Cooling (20° C./hr) | EtOH/5.0 | ppt/filter | Weak crystalline |
| MeOH/0.5 | 60.0 | Slow Cooling (20° C./hr) | n-PrOH/5.0 | ppt/filter | Weak crystalline |
| MeOH/0.5 | 60.0 | Slow Cooling (20° C./hr) | IPA/2.5 | ppt/filter | A |
| MeOH/0.5 | 60.0 | Slow Cooling (20° C./hr) | n-BuOH/5.0 | ppt/filter | Weak crystalline |
| MeOH/0.5 | 60.0 | Slow Cooling (20° C./hr) | MeCN/2.5 | ppt/filter | Weak crystalline |
| MeOH/0.5 | 60.0 | Slow Cooling (20° C./hr) | THF/0.5 | ppt/filter | Weak crystalline |
| MeOH/0.5 | 60.0 | Slow Cooling (20° C./hr) | 2-MeTHF/0.1 | ppt/filter | A |
| MeOH/0.5 | 60.0 | Slow Cooling (20° C./hr) | EtOAc/0.2 | ppt/filter | A |
| MeOH/0.5 | 60.0 | Slow Cooling (20° C./hr) | IPAc/0.1 | ppt/filter | A |
| MeOH/0.5 | 60.0 | Slow Cooling (20° C./hr) | Acetone/0.5 | ppt/filter | Weak crystalline |
| MeOH/0.5 | 60.0 | Slow Cooling (20° C./hr) | MEK/0.2 | ppt/filter | A |
| MeOH/0.5 | 60.0 | Slow Cooling (20° C./hr) | MIBK/0.1 | ppt/filter | A |
| MeOH/0.5 | 60.0 | Slow Cooling (20° C./hr) | DCM/5.0 | Clear/Evap. | A |
| MeOH/0.5 | 60.0 | Slow Cooling (20° C./hr) | Toluene/1.5 | ppt/filter | Weak |
| | | | | | crystalline |
| MeOH/0.5 | 60.0 | Slow Cooling (20° C./hr) | MTBE/0.1 | ppt/filter | A |

TABLE 4
Slurry Experiments of Compound 2

| | Solvent | | Time point | Time point |
|---|---|---|---|---|
| | Vol. | | (1 day) | (7 days) |
| Solvent | (mL) | Method | XRPD | XRPD |
| IPA | 1.0 | Stirring at RT | A | F |
| MeCN | 1.0 | Stirring at RT | D | D |
| THF | 1.0 | Stirring at RT | Weak | E |
| | | | Crystalline | |
| 2-MeTHF | 1.0 | Stirring at RT | Weak | B |
| | | | Crystalline | |
| EtOAc | 1.0 | Stirring at RT | A | C |
| IPAc | 1.0 | Stirring at RT | A with extra | B |
| | | | peak | |
| Acetone | 1.0 | Stirring at RT | E | B |
| MEK | 1.0 | Stirring at RT | Weak | B |
| | | | Crystalline | |
| MIBK | 1.0 | Stirring at RT | E | B |
| Toluene | 1.0 | Stirring at RT | E | B |
| MTBE | 1.0 | Stirring at RT | A | B |
| n-Heptane | 1.0 | Stirring at RT | A | A |
| c-Hexane | 1.0 | Stirring at RT | A | A |

A summary of characterization data of all isolated forms of Compound IA is given in Table 5. Forms A, B, and D were evaluated as solid state forms.

TABLE 5
Characterization Data of Morphic Forms of Compound IA

| XRPD | Possible | | | 1H NMR | % C1 |
|---|---|---|---|---|---|
| Pattern | Form | DSC (° C.) | TGA (wt loss) | (DMSO-d₆) | (API:HCl) |
| A | Hydrate | Endotherms | Onset 5.7 wt % loss at | Contains | 11.1% |
| | | at 110.3, | 66.0° C., Onset 5.4 wt % loss | water | (1:1.67)* |
| | | 275.6, 344.8 | at 215.5° C., Onset 6.2 wt % | | |
| | | | loss at 314.0° C. | | |
| B | Hydrate | Endotherms | Onset 5.1 wt % loss at | Contains | 11.90% |
| | | at 105.2, | 60.9° C., Onset 7.2 wt % loss | water and | (1:1.81) |
| | | 220.8, 265.6, | at 198.3° C., Onset 7.8 wt % | residual | |
| | | 350.6 | loss at 319.6° C. | solvent | |
| C | EtOAc | Endotherms | Onset 1.6 wt % loss at | Contains | Not |
| | solvate | at 95.1, | 72.9° C., Onset 5.1 wt % loss | water and | determined |
| | | 235.6, 257.8, | at 192.0° C., Onset 0.9 wt % | EtOAc as | |
| | | 344.6 | loss at 223.4° C., Onset 6.9 | residual | |
| | | | wt % loss at 306.7° C. | solvent | |
| D | Hydrate | Endotherms | Onset 6.0 wt % loss at | Contains | 12.23% |
| | | at 108.3, | 68.8° C., Onset 6.0 wt % loss | water and | (1:1.87) |
| | | 266.1, 347.0 | at 207.6° C., Onset 3.6 wt % | residual | |
| | | | loss at 304.9° C., Onset 6.6 | solvent | |
| | | | wt % loss at 324.7° C. | | |
| E | Acetone | Endotherms | Onset 1.0 wt % loss at | Contains | Not |
| | solvate | at 70.3, | 41.9° C., Onset 1.1 wt % loss | water and | determined |
| | | 275.2, 345.9 | at 61.5° C., Onset 1.0 wt % | acetone as | |
| | | Exotherm at | loss at 93.2° C., Onset 5.0 | residual | |
| | | 220.0 | wt % loss at 211.6° C., Onset | solvent | |
| | | | 5.6 wt % loss at 308.5° C. | | |
| F | Unstable | Endotherms | Onset 8.0 wt % loss at | Contains | Not |
| | hydrate | at 73.2, | 43.7° C., Onset 2.1 wt % loss | water | determined |
| | | 214.5, 303.4, | at 190.7° C., Onset 7.6 wt % | | |
| | | 329.7 | loss at 308.8° C. | | |
| | | Exotherm at | | | |
| | | 277.8 | | | |
| G | Anhydrate | Endotherms | Onset 4.5 wt % loss at | Contains | Not |
| | | at 81.8, | 47.2° C., Onset 3.1 wt % loss | water | determined |
| | | 120.8, 268.2, | at 86.6° C., Onset 4.5 wt % | | |
| | | 347.9 | loss at 213.3° C., Onset 4.6 | | |
| | | | wt % loss at 311.2° C. | | |
| H | n-PrOH | Endotherms | Onset 1.9 wt % loss at | Contains | Not |
| | solvate | at 110.5, | 45.6° C., Onset 4.6 wt % loss | water and | determined |
| | | 225.6, 274.5, | at 71.9° C., Onset 1.8 wt % | n-PrOH as | |
| | | 346.3 | loss at 187.9° C., Onset 2.2 | residual | |
| | | | wt % loss at 222.1° C., Onset | solvent | |
| | | | 3.0 wt % loss at 303.0° C., | | |
| | | | Onset 2.2 wt % loss at | | |
| | | | 325.2° C. | | |

In one embodiment Form A is characterized by at least one XRPD peaks at 7.4±0.2°, 9.0±0.2°, or 12.3±0.2° 2theta. In one embodiment Form B is characterized by at least one XRPD peaks at 6.4±0.2°, or 9.5±0.2° 2theta. In one embodiment Form C is characterized by at least one XRPD peaks at 5.3±0.2°, or 7.2±0.2° 2theta. In one embodiment Form D is characterized by at least one XRPD peaks at 5.6±0.2°, or 8.2±0.2° 2theta. In one embodiment Form E is characterized by at least one XRPD peak at 5.5±0.2°, or 6.7±0.2° 2theta. In one embodiment Form E is characterized by at least one XRPD peak at 5.5±0.2°, or 6.7±0.2° 2theta. In one embodiment Form F is characterized by a XRPD peak at 7.2±0.2° 2theta. In one embodiment Form G is characterized by a XRPD peak at 6.7±0.2° 2theta. In one embodiment Form H is characterized by a XRPD peak at 6.6±0.2° 2theta.

### Example 13. Recrystallization Procedures to Produce Form B from Compound 2

Recrystallization studies were conducted to define a procedure to improve chromatographic purity. All recrystallization procedures in Table 6 involved dissolving Compound IA in concentrated HCl and then adding the anti-solvent, acetone. The differences in the processes are subtle but important in terms of their results.

Recrystallization Process 1:

Compound I was charged to an appropriately sized flask or reactor, dissolved in aqueous hydrochloric acid solution and heated to at least 55±10° C. The solution was stirred for about 45 minutes and the resulting mixture was filtered through an in-line filter. Acetone was added at 55±10° C. over the course of an hour and the solution was stirred for about an additional hour. The temperature was decreased to about 25±5° C., and the solution was stirred for at least 2 hours. The solids were collected by filtration, washed with acetone, and dried to afford Compound IA Form B.

Recrystallization Process 2:

Compound I was charged to an appropriately sized flask or reactor, dissolved in aqueous hydrochloric acid solution and heated to at least 55±10° C. The solution was stirred for about 45 minutes and the resulting mixture was filtered through an in-line filter. The temperature was decreased to about 25±5° C., and the solution was stirred for at least 2 hours. Acetone was added at 25±5° C. over the course of an hour and the solution was stirred for an additional two hours. The solids were collected by filtration, washed with acetone, and dried to afford Compound IA form D.

Recrystallization Process 3:

Compound I was charged to an appropriately sized flask or reactor, dissolved in aqueous hydrochloric acid solution and heated to at least 55±10° C. The solution was stirred for about 45 minutes and the resulting mixture was filtered through an in-line filter. The temperature was decreased to about 25±5° C. and the solution was stirred for at least 2 hours. The solids were collected by filtration, washed with acetone, and dried to afford Compound IA form D.

TABLE 6
Effect of crystallization procedures on purging
of chromatographic impurities from Compound 1

| | | Recrys | Recrys | Recrys |
|---|---|---|---|---|
| | | Process 1 | Process 2 | Process 3 |
| RRT | % area | % area | % area | % area |
| 1.11 | 1.13 | 1.11 | 0.87 | 0.27 |
| 1.37 | 0.14 | 0.15 | 0.13 | ND |
| 1.62 | 0.14 | ND | 0.13 | ND |

While conducting the experiments presented in Table 6, it was discovered that not all recrystallization processes resulted in the preferred solid state form, Form B. Specifically, Recrystallization Processes 2 and 3 result in a different solid state form (putative Form D) whereas Recrystallization 1 reproducibly provides Form B. In some embodiments, Compound IA is converted to Form D by Recrystallization Procedure 2 and 3 and Form D is converted to Form B by Recrystallization Process 1.

### Example 14. XRPD Analysis of Compound IA, Morphic Form B

The XRPD pattern of Form B was collected with a PANalytical X'Pert PRO MPD diffractometer using an incident beam of Cu radiation produced using an Optix long, fine-focus source. An elliptically graded multilayer mirror was used to focus Cu Kα X-rays through the specimens and onto the detector. Prior to the analysis, a silicon specimen (NIST SRM 640e) was analyzed to verify the observed position of the Si 111 peak is consistent with the NIST-certified position. The sample was sandwiched between 3-μm-thick films and analyzed in transmission geometry. A beamstop, short anti-scatter extension and an anti-scatter knife edge were used to minimize the background generated by air. Soller slits for the incident and diffracted beams were used to minimize broadening from axial divergence. The diffraction patterns were collected using a scanning position-sensitive detector (X'Celerator) located 240 mm from the specimens and Data Collector software v. 2.2b. Data acquisition parameters for each pattern are displayed above the image in the Data section of this report including the divergence slit (DS) before the mirror.

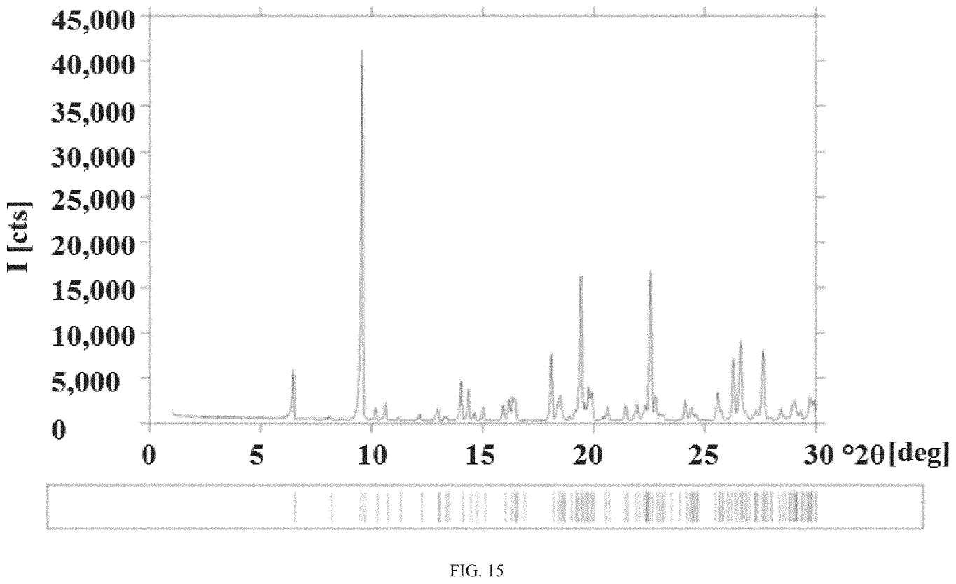

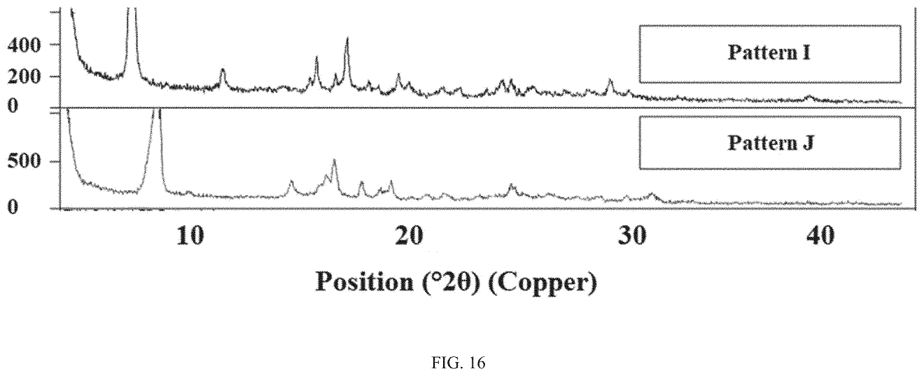

The XRPD pattern of pure Form B along with the indexing solution is shown in FIG. 15. The pure Form B XRPD pattern exhibited sharp peaks, indicating the sample was composed of crystalline material. The allowed peak positions from the XRPD indexing solution are 6.5, 8.1, 9.4, 9.6, 10.2, 10.6, 11.2, 12.2, 12.9, 13.0, 13.3, 13.4, 14.0, 14.4, 14.6, 15.0, 15.9, 16.2, 16.4, 16.5, 16.8, 18.1, 18.4, 18.5, 18.6, 18.6, 18.9, 19.1, 19.2, 19.3, 19.4, 19.5, 19.6, 19.7, 19.8, 19.9, 20.4, 20.6, 21.3, 21.4, 21.8, 22.0, 22.2, 22.3, 22.4, 22.5, 22.8, 23.0, 23.1, 23.4, 23.8, 24.1, 24.2, 24.3, 24.4, 24.5, 24.6, 25.4, 25.6, 25.7, 25.9, 26.0, 26.1, 26.3, 26.4, 26.5, 26.6, 26.7, 26.8, 26.9, 27.2, 27.3, 27.5, 27.6, 27.7, 27.9, 28.3, 28.4, 28.5, 28.7, 28.9, 29.0, 29.1, 29.3, 29.4, 29.5, 29.6, 29.7, 29.8, 29.9, 30.0, 30.3, 30.4, 30.5, 30.6, 30.7, 30.9, 31.2, 31.5, 31.6, 31.7, 31.8, 31.9, 32.0, 32.2, 32.3, 32.4, 32.5, 32.6, 32.7, 32.8, 33.1, 33.2, 33.3, 33.6, 33.7, 33.8, 34.0, 34.1, 34.2, 34.3, 34.6, 34.7, 34.8, 35.0 35.2, 35.3, 35.5, 35.6, 35.9, 36.0, 36.2, 36.5, 36.6, 36.7, 36.8, 36.9, 37.1, 37.2, 37.3, 37.4, 37.5, 37.6, 37.7, 37.8, 37.9, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, and 40.0°2θ.

For example, Form B's XRPD may be indexed as follows 6.47, 8.08, 9.42, 9.59, 10.18, 10.62, 11.22, 12.17, 12.91, 12.97, 13.27, 13.37, 14.03, 14.37, 14.63, 15.02, 15.93, 16.20, 16.35, 16.43, 16.47, 16.81, 18.10, 18.35, 18.41, 18.50, 18.55, 18.60, 18.91, 19.11, 19.15, 19.24, 19.34, 19.43, 19.51, 19.61, 19.65, 19.76, 19.85, 19.90, 20.44, 20.61, 21.34, 21.43, 21.84, 21.95, 22.17, 22.28, 22.30, 22.33, 22.44, 22.54, 22.76, 22.81, 22.97, 23.00, 23.11, 23.42, 23.80, 24.11, 24.22, 24.34, 24.38, 24.40, 24.48, 24.56, 24.57, 25.40, 25.56, 25.57, 25.59, 25.72, 25.74, 25.94, 25.99, 26.11, 26.28, 26.29, 26.37, 26.51, 26.58, 26.61, 26.73, 26.81, 26.92, 27.15, 27.19, 27.23, 27.31, 27.49, 27.57, 27.61, 27.71, 27.88, 27.94, 28.27, 28.41, 28.53, 28.71, 28.74 28.86, 28.94, 28.98, 29.03, 29.06, 29.08, 29.25, 29.30, 29.38, 29.51, 29.57, 29.61, 29.70, 29.73, 29.75, 29.90, 29.95, 30.31, 30.38, 30.42, 30.54, 30.55, 30.66, 30.73, 30.85, 30.87, 30.89, 31.23, 31.51, 31.55, 31.61, 31.70, 31.76, 31.77, 31.80, 31.81, 31.82, 31.82, 31.90, 31.91, 31.95, 32.17, 32.21, 32.23, 32.25, 32.36, 32.37, 32.43, 32.53, 32.54, 32.56, 32.61, 32.73, 32.80, 32.82, 33.05, 33.13, 33.17, 33.22, 33.28, 33.30, 33.60, 33.65, 33.71, 33.76, 33.77, 33.99, 34.01, 34.01, 34.05, 34.10, 34.17, 34.29, 34.55, 34.60, 34.62, 34.63, 34.68, 34.75, 34.76, 35.03, 35.16, 35.19, 35.21, 35.25, 35.31, 35.46, 35.61, 35.63, 35.85, 35.86, 35.90, 35.97, 36.19, 36.45, 36.56, 36.58, 36.67, 36.68, 36.70, 36.71, 36.77, 36.85, 36.87, 36.90, 37.09, 37.19, 37.27, 37.28, 37.29, 37.32, 37.33, 37.37, 37.38, 37.48, 37.48, 37.50, 37.51, 37.54, 37.61, 37.64, 37.65, 37.68, 37.69, 37.71, 37.74, 37.74, 37.76, 37.81, 37.83, 37.93, 37.94, 38.15, 38.19, 38.32, 38.36, 38.39, 38.46, 38.59, 38.63, 38.69, 38.76, 38.79, 38.85, 38.87, 38.88, 38.96, 38.98, 39.02, 39.05, 39.19, 39.27, 39.33, 39.36, 39.39, 39.43, 39.44, 39.53, 39.53, 39.6, 39.61, 39.70, 39.71, 39.72, 39.82, 39.87, 39.9, and 39.98°2θ.

Observed peaks for Form B include 9.5±0.2, 18.1±0.2, 19.3±0.2, 22.4±0.2, 26.6±0.2, and 27.7±0.2, °2θ.

Agreement between the allowed peak positions, marked with bars, and the observed peaks indicated a consistent unit cell determination. Successful indexing of the pattern indicated that the sample was composed primarily of a single crystalline phase. Space groups consistent with the assigned extinction symbol, unit cell parameters, and derived quantities are given in Table 7.

TABLE 7
Parameters of the XRPD of Compound IA, Form B

| Bravais Type | C-centered Monoclinic |
|---|---|
| a [Å] | 27.719 |
| b [Å] | 9.796 |
| c [Å] | 22.221 |
| α [deg] | 90 |
| β [deg] | 100.16 |
| γ [deg] | 90 |
| Volume [Å³/cell] | 5,939.0 |
| Chiral contents | Not specified |
| Extinction Symbol | C 1 c 1 |
| Space Group(s) | Cc (9), C2/c(15) |

In some embodiments, Form B is characterized by an XRPD pattern comprising at least two 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising at least three 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising at least four 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising at least five 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising at least six 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising the 2theta values selected from 6.5±0.2°, 9.5±0.2°, 14.0±0.2°, 14.4±0.2°, 18.1±0.2°, 19.9±0.2°, and 22.4±0.2°. In some embodiments, Form B is characterized by an XRPD pattern comprising at least the 2theta value of 9.5±0.4°.

This specification has been described in reference to embodiments of the invention. However, one of ordinary skill in the art appreciates that various modification and changes can be made without departing from the scope of the invention as set forth in the claims below. Accordingly, the specification is to be regarded in an illustrative rather than restrictive sense, and all such modifications are intended to be included within the scope of the invention.

1.-60. (canceled)

61. A method of treating a human with non-small cell lung cancer, the method comprising administering to the human an effective amount of a selective Cyclin Dependent Kinase 4/6 inhibitor compound of the structure: 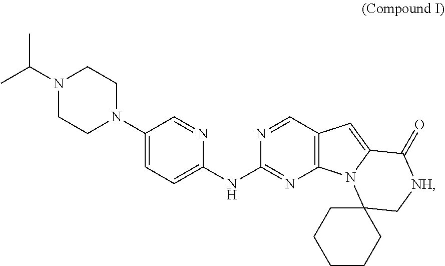 [SMILES: CC(C)N1CCN(c2ccc(Nc3ncc4cc5n(c4n3)C3(CCCCC3)CNC5=O)nc2)CC1]
or a pharmaceutically acceptable salt thereof, and administering to the human an effective amount of an extracellular signal-regulated kinase (ERK) inhibitor,
wherein the NSCLC harbors a mutation selected from the group consisting of a Kristen rat sarcoma viral oncogene homolog (KRAS) mutation, estrogen-related receptor beta type 2 (ERBB2)-amplification, anaplastic lymphoma kinase (ALK) fusion, and MET amplification or mutation.

62. The method of claim 61, wherein the ERK inhibitor is selected from the group consisting of ulixertinib and SCH772984.

63. The method of claim 61, wherein the NSCLC is Retinoblastoma protein-null.

64. The method of claim 61, wherein the NSCLC is epithelial NSCLC and the KRAS mutation is selected from the group consisting of a G12S substitution, G12D substitution, G12C substitution, G12A substitution, G12V substitution, and Q61K substitution.

65. The method of claim 61, wherein the ALK fusion is an EML4-ALK rearrangement.

66. The method of claim 61, wherein the MET amplification or mutation is a MET exon 14 deletion.

67. The method of claim 61, wherein the NSCLC has an ERBB-2 amplification and is resistant to an EGFR inhibitor.

68. The method of claim 61, wherein the treatment further comprises administering to the human a MEK inhibitor.

69. The method of claim 68, wherein the MEK inhibitor is selected from the group consisting of selumetinib, trametinib, binimetinib, and cobimetinib.

70. The method of claim 61, wherein the CDK4/6 inhibitor and ERK inhibitor are administered at least once a day.

71. The method of claim 70, wherein the CDK4/6 inhibitor and ERK inhibitor are administered at least once a day for 28 days or more.

72. The method of claim 61, wherein the CDK4/6 inhibitor compound is:

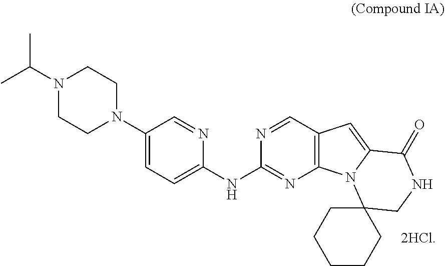 [SMILES: CC(C)N1CCN(c2ccc(Nc3ncc4cc5n(c4n3)C3(CCCCC3)CNC5=O)nc2)CC1]

73. The method of claim 61, wherein the CDK4/6 inhibitor is morphic form B, characterized by an XPRD pattern comprising at least three 2theata values selected from 6.5±0.2°, 9.5±0.2°, 14.0±0.2°, 14.4±0.2°, 18.1±0.2°, 19.9±0.2°, and 22.4±0.2°.

74. A method of treating a human with non-small cell lung cancer, the method comprising administering to the human an effective amount of a selective Cyclin Dependent Kinase 4/6 inhibitor compound of the structure: 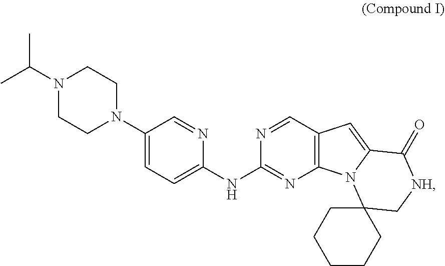 [SMILES: CC(C)N1CCN(c2ccc(Nc3ncc4cc5n(c4n3)C3(CCCCC3)CNC5=O)nc2)CC1]
or a pharmaceutically acceptable salt thereof, and administering to the human an effective amount of an ERK inhibitor and a MEK inhibitor,
wherein the NSCLC harbors a KRAS mutation.

75. The method of claim 74, wherein the ERK inhibitor is selected from the group consisting of ulixertinib and SCH772984.

76. The method of claim 74, wherein the NSCLC is Retinoblastoma protein-null.

77. The method of claim 74, wherein the NSCLC is epithelial NSCLC and the KRAS mutation is selected from the group consisting of a G12S substitution, G12D substitution, G12C substitution, G12A substitution, G12V substitution, and Q61K substitution.

78. The method of claim 74, wherein the MEK inhibitor is selected from the group consisting of selumetinib, trametinib, binimetinib, and cobimetinib.

79. The method of claim 74, wherein the ERK inhibitor is ulixertinib and that MEK inhibitor is selumetinib.

80. The method of claim 74, wherein the CDK4/6 inhibitor and ERK inhibitor and MEK inhibitor are administered at least once a day.

81. The method of claim 80, wherein the CDK4/6 inhibitor and ERK inhibitor and MEK inhibitor are administered at least once a day for 28 days or more.

82. The method of claim 74, wherein the CDK4/6 inhibitor compound is:

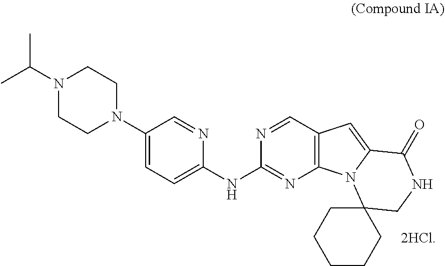 [SMILES: CC(C)N1CCN(c2ccc(Nc3ncc4cc5n(c4n3)C3(CCCCC3)CNC5=O)nc2)CC1]

83. The method of claim 74, wherein the CDK4/6 inhibitor is morphic form B, characterized by an XPRD pattern comprising at least three 2theata values selected from 6.5±0.2°, 9.5±0.2°, 14.0±0.2°, 14.4±0.2°, 18.1±0.2°, 19.9±0.2°, and 22.4±0.2°.

84. A method of treating a human with non-small cell lung cancer, the method comprising administering to the human an effective amount of a selective Cyclin Dependent Kinase 4/6 inhibitor compound of the structure: 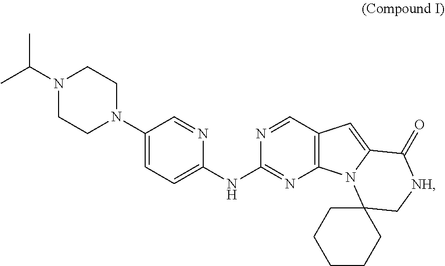 [SMILES: CC(C)N1CCN(c2ccc(Nc3ncc4cc5n(c4n3)C3(CCCCC3)CNC5=O)nc2)CC1]
or a pharmaceutically acceptable salt thereof, and administering to the human an effective amount of a RET inhibitor,
wherein the cancer has a RET mutation or RET fusion.

85. The method of claim 84, wherein the non-small cell cancer has a RET fusion.

86. The method of claim 85, wherein the RET fusion is selected from the group consisting of CCDC6-RET, KIF5B-RET, and TRIM22-RET.

87. The method of claim 84, wherein the RET inhibitor is selected from the group consisting of pralsetinib, agerafenib, lenvatinib, apatinib, and LOXO-292.

88. The method of claim 84, wherein the CDK4/6 inhibitor and RET inhibitor are administered at least once a day.

89. The method of claim 88, wherein the CDK4/6 inhibitor and RET inhibitor are administered at least once a day for 28 days or more.

90. The method of claim 84, wherein the CDK4/6 inhibitor compound is:

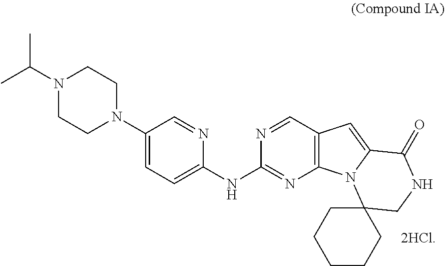 [SMILES: CC(C)N1CCN(c2ccc(Nc3ncc4cc5n(c4n3)C3(CCCCC3)CNC5=O)nc2)CC1]

91. The method of claim 84, wherein the CDK4/6 inhibitor is morphic form B, characterized by an XPRD pattern comprising at least three 2theata values selected from 6.5±0.2°, 9.5±0.2°, 14.0±0.2°, 14.4±0.2°, 18.1±0.2°, 19.9±0.2°, and 22.4±0.2°.

92. A method of treating a human with KRAS mutant non-small cell lung cancer, the method comprising administering to the human an effective amount of a selective Cyclin Dependent Kinase 4/6 inhibitor compound of the structure: 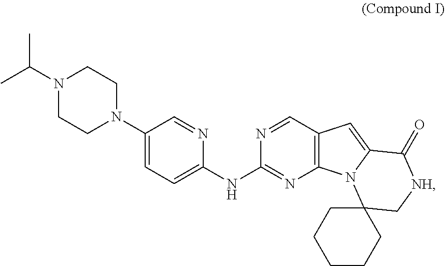 [SMILES: CC(C)N1CCN(c2ccc(Nc3ncc4cc5n(c4n3)C3(CCCCC3)CNC5=O)nc2)CC1]
or a pharmaceutically acceptable salt thereof, and administering to the human an effective amount of an ERK inhibitor,
wherein the non-small cell lung cancer is KRAS mutant epithelial non-small cell lung cancer.

93. The method of claim 92, wherein the KRAS mutation is selected from the group consisting of a G12S substitution, G12D substitution, G12C substitution, G12A substitution, G12V substitution, and Q61K substitution.

94. The method of claim 92, wherein the non-small cell lung cancer is retinoblastoma-protein null.

95. The method of claim 92, wherein the ERK inhibitor is selected from the group consisting of ulixertinib and SCH772984.

96. The method of claim 92, wherein the CDK4/6 inhibitor and the ERK inhibitor are administered at least once a day.

97. The method of claim 96, wherein the CDK4/6 inhibitor and the ERK inhibitor are administered at least once a day for 28 days or more.

98. The method of claim 92, wherein the CDK4/6 inhibitor compound is:

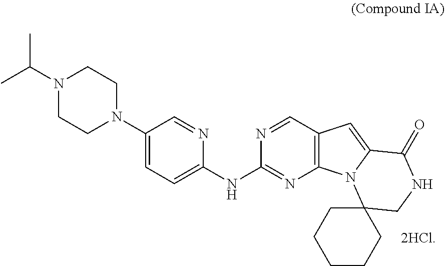 [SMILES: CC(C)N1CCN(c2ccc(Nc3ncc4cc5n(c4n3)C3(CCCCC3)CNC5=O)nc2)CC1]

99. The method of claim 92, wherein the CDK4/6 inhibitor is morphic form B, characterized by an XPRD pattern comprising at least three 2theata values selected from 6.5±0.2°, 9.5±0.2°, 14.0±0.2°, 14.4±0.2°, 18.1±0.2°, 19.9±0.2°, and 22.4±0.2°.

100. A method of treating a human with non-small cell lung cancer, the method comprising administering to the human an effective amount of a selective Cyclin Dependent Kinase 4/6 inhibitor compound of the structure: 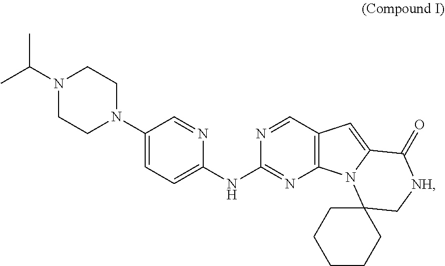 [SMILES: CC(C)N1CCN(c2ccc(Nc3ncc4cc5n(c4n3)C3(CCCCC3)CNC5=O)nc2)CC1]
or a pharmaceutically acceptable salt thereof, and administering to the human an effective amount of an ALK inhibitor,
wherein the non-small cell lung cancer has an ALK fusion.

101. The method of claim 100, wherein the ALK fusion is selected from the group consisting of an EML4-ALK fusion a KIFSB-ALK fusion, a TFG-ALK fusion.

102. The method of claim 100, wherein the non-small cell lung cancer is retinoblastoma-protein null.

103. The use according to claim 100, wherein the ALK inhibitor is selected from the group consisting of crizotinib, alectinib, lorlatinib, entrectinib, brigatinib, and ceritinib.

104. The method of claim 100, wherein the CDK4/6 inhibitor and the ALK inhibitor are administered at least once a day.

105. The method of claim 104, wherein the CDK4/6 inhibitor and the ALK inhibitor are administered at least once a day for 28 days or more.

106. The method of claim 100, wherein the CDK4/6 inhibitor compound is:

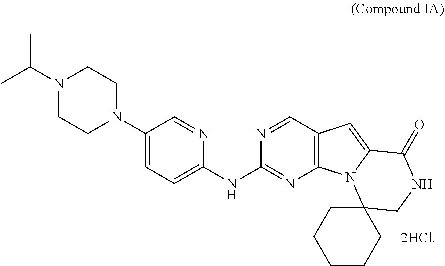 [SMILES: CC(C)N1CCN(c2ccc(Nc3ncc4cc5n(c4n3)C3(CCCCC3)CNC5=O)nc2)CC1]

107. The method of claim 100, wherein the CDK4/6 inhibitor is morphic form B, characterized by an XPRD pattern comprising at least three 2theata values selected from 6.5±0.2°, 9.5±0.2°, 14.0±0.2°, 14.4±0.2°, 18.1±0.2°, 19.9±0.2°, and 22.4±0.2°.

108. A method of treating a human with non-small cell lung cancer, the method comprising administering to the human an effective amount of a selective Cyclin Dependent Kinase 4/6 inhibitor compound of the structure: 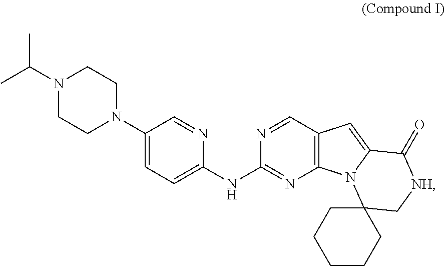 [SMILES: CC(C)N1CCN(c2ccc(Nc3ncc4cc5n(c4n3)C3(CCCCC3)CNC5=O)nc2)CC1]
or a pharmaceutically acceptable salt thereof, and administering to the human an effective amount of an additional kinase inhibitor selected from the group consisting of an ERK inhibitor, an ALK inhibitor, and a PI3K inhibitor, or a combination thereof,
wherein the non-small cell lung cancer has a MET amplification or mutation.

109. The method of claim 108, wherein the MET amplification or mutation is a MET exon 14 deletion.

110. The method of claim 108, wherein the non-small cell lung cancer is retinoblastoma-protein null.

111. The method of claim 108, wherein the ERK inhibitor is selected from the group consisting of ulixertinib and SCH772984.

112. The method of claim 108, wherein the ALK inhibitor is crizotinib.

113. The method of claim 108, wherein the PI3K inhibitor is selected from the group consisting of dactolisib, idealisib, copanlisib, taselisib, perifosine, buparlisib, duvelisib, alpelisib, and umbralisib.

114. The method of claim 108, wherein the CDK4/6 inhibitor and the ERK, ALK, or PI3K inhibitor are administered at least once a day.

115. The method of claim 114, wherein the CDK4/6 inhibitor and the ERK, ALK, or PI3K inhibitor are administered at least once a day for 28 days or more.

116. The method of claim 108, wherein the CDK4/6 inhibitor compound is:

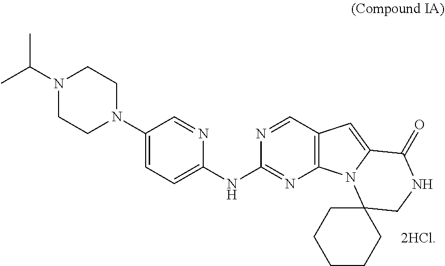 [SMILES: CC(C)N1CCN(c2ccc(Nc3ncc4cc5n(c4n3)C3(CCCCC3)CNC5=O)nc2)CC1]

117. The method of claim 108, wherein the CDK4/6 inhibitor is morphic form B, characterized by an XPRD pattern comprising at least three 2theata values selected from 6.5±0.2°, 9.5±0.2°, 14.0±0.2°, 14.4±0.2°, 18.1±0.2°, 19.9±0.2°, and 22.4±0.2°.
